SEQUENCE LISTING (48 sequences)

SEQ ID NO: 1 - protein, 207 aa, Homo sapiens
  SIGNAL 1..21
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 2 - protein, 188 aa, Homo sapiens [sequence omitted]

SEQ ID NO: 3 - protein, 119 aa, synthetic construct
VQLQQPGTELVKPGASVKLSCKASGYTFTGFWIHWVKQRPGQGLEWIGHINPGNGGTNYNEKFKRMATLTVDKSSSTAYMQLSSLTSEDSAVYYCARSYSNYVRAMDYWGQGTSVTVSS

SEQ ID NO: 4 - protein, 106 aa, synthetic construct
IQMTQTTSSLSASLGDRVTISCRASQDISNFLNWYQQKPDGTVKLLIYYTSTLHSGVPSRFSGSGSGTDYSLTISNLEQEDIATYFCQQGKTLPPTFGGGTKLEIK

SEQ ID NO: 5 - protein, 119 aa, synthetic construct
VQLVQSGAEVKKPGASVKVSCKASGYTFTGFWIHWVRQAPGQGLEWMGHINPGNGGTNYNEKFKRRVTMTRDKSISTAYMELSRLRSDDTAVYYCARSYSNYVRAMDYWGQGTLVTVSS

SEQ ID NO: 6 - protein, 107 aa, synthetic construct
DIQMTQSPSSLSASVGDRVTITCRASQDISNFLNWYQQKPGKAPKLLIYYTSTLHSGVPSRFSGSGSGTDFTLTISSLQPEDFATYYCQQGKTLPPTFGGGTKVEIK

SEQ ID NO: 7 - protein, 116 aa, synthetic construct
VQPQQPGAELVKPGASVKMSCKASGDTFTNSWIGWVTQRPGQGLEWIGDIFPGSGHTNYNEKFKNRATLTVDTSSSTAYMLLSSLTSDDSAVYYCVIENYAWFAYWGQGTLVTVSA

SEQ ID NO: 8 - protein, 107 aa, synthetic construct
DIVMTQSQKFMSSSVGDRVSVTCKASQNVNTNVAWYQQKQGQSPRPLIYSASSRCSGVPDRFTGSGFGTDFTLTISNVQSEDLAEYFCQQYHSFPLTFGAGAKLDLK

SEQ ID NO: 9 - protein, 118 aa, synthetic construct
VQLQQSGPELVKPGTSVRISCKASGYTFTTFYIHWVKQRPGQGLEWIGWFYPGNVNTNYNEKLKGKATLTADKSSSAAYLQLNSLTSEDSAVYFCTRSPYYGYVFDYWGQGTTLTVSS

SEQ ID NO: 10 - protein, 107 aa, synthetic construct
DIVMTQSHKFMSTSVGDRVSITCKASQDVGTAVAWYQQKPGQSPKLLIYWASTRHTGVPDRFTGSGSGTDFTLTISNVQSEDLADYFCQQYSSSLTFGAGATKLELK

SEQ ID NO: 11 - DNA, 33 bp, synthetic construct
  source 1..33: mol_type = other DNA
agggcaagtcaggacattagcaattttttaaac

SEQ ID NO: 12 - DNA, 21 bp, synthetic construct
  source 1..21: mol_type = other DNA
tacacatcaacattacactca

SEQ ID NO: 13 - DNA, 27 bp, synthetic construct
  source 1..27: mol_type = other DNA
caacagggtaaaacgcttcctcccacg

SEQ ID NO: 14 - DNA, 30 bp, synthetic construct
  source 1..30: mol_type = other DNA
ggctacactttcactggcttctggatacac

SEQ ID NO: 15 - DNA, 51 bp, synthetic construct
  source 1..51: mol_type = other DNA
catattaatcctggcaatggtggcactaactacaatgagaagttcaagaga

SEQ ID NO: 16 - DNA, 33 bp, synthetic construct
  source 1..33: mol_type = other DNA
tcctatagtaactacgtgcgggctatggactac

SEQ ID NO: 17 - protein, 11 aa, synthetic construct
RASQDISNFLN

SEQ ID NO: 18 - protein, 7 aa, synthetic construct
YTSTLHS

SEQ ID NO: 19 - protein, 9 aa, synthetic construct
QQGKTLPPT

SEQ ID NO: 20 - protein, 10 aa, synthetic construct
GYTFTGFWIH

SEQ ID NO: 21 - protein, 17 aa, synthetic construct
HINPGNGGTNYNEKFKR

SEQ ID NO: 22 - protein, 11 aa, synthetic construct
SYSNYVRAMDY

SEQ ID NO: 23 - DNA, 33 bp, synthetic construct
  source 1..33: mol_type = other DNA
aaggccagtcaggatgtgggtactgctgtagcc

SEQ ID NO: 24 - DNA, 21 bp, synthetic construct
  source 1..21: mol_type = other DNA
tgggcatccacccggcacact

SEQ ID NO: 25 - DNA, 24 bp, synthetic construct
  source 1..24: mol_type = other DNA
caacaatatagcagctctctcacg

SEQ ID NO: 26 - DNA, 30 bp, synthetic construct
  source 1..30: mol_type = other DNA
ggctacaccttcacaaccttctatatacac

SEQ ID NO: 27 - DNA, 51 bp, synthetic construct
  source 1..51: mol_type = other DNA
tggttttatcctggaaatgttaataccaactacaatgagaagctcaagggc

SEQ ID NO: 28 - DNA, 30 bp, synthetic construct
  source 1..30: mol_type = other DNA
tccccttactacggctacgtttttgactac

SEQ ID NO: 29 - protein, 11 aa, synthetic construct
KASQDVGTAVA

SEQ ID NO: 30 - protein, 7 aa, synthetic construct
WASTRHT

SEQ ID NO: 31 - protein, 8 aa, synthetic construct
QQYSSSLT

SEQ ID NO: 32 - protein, 10 aa, synthetic construct
GYTFTTFYIH

SEQ ID NO: 33 - protein, 17 aa, synthetic construct
WFYPGNVNTNYNEKLKG

SEQ ID NO: 34 - protein, 10 aa, synthetic construct
SPYYGYVFDY

SEQ ID NO: 35 - DNA, 33 bp, synthetic construct
  source 1..33: mol_type = other DNA
aaggccagtcagaatgtgaacactaatgtagcc

SEQ ID NO: 36 - DNA, 21 bp, synthetic construct
  source 1..21: mol_type = other DNA
tcggcatcctcccggtgcagt

SEQ ID NO: 37 - DNA, 27 bp, synthetic construct
  source 1..27: mol_type = other DNA
cagcaatatcacagctttccgctcacg

SEQ ID NO: 38 - DNA, 30 bp, synthetic construct
  source 1..30: mol_type = other DNA
ggcgacaccttcaccaactcctggataggc

SEQ ID NO: 39 - DNA, 51 bp, synthetic construct
  source 1..51: mol_type = other DNA
gatatttttcctgggagtggtcatactaactacaatgagaagttcaagaac

SEQ ID NO: 40 - DNA, 24 bp, synthetic construct
  source 1..24: mol_type = other DNA
gagaattatgcctggtttgcttat

SEQ ID NO: 41 - protein, 11 aa, synthetic construct
KASQNVNTNVA

SEQ ID NO: 42 - protein, 7 aa, synthetic construct
SASSRCS

SEQ ID NO: 43 - protein, 9 aa, synthetic construct
QQYHSFPLT

SEQ ID NO: 44 - protein, 10 aa, synthetic construct
GDTFTNSWIG

SEQ ID NO: 45 - protein, 17 aa, synthetic construct
DIFPGSGHTNYNEKFKN

SEQ ID NO: 46 - protein, 8 aa, synthetic construct
ENYAWFAY

SEQ ID NO: 47 - protein, 214 aa, synthetic construct
DIQMTQSPSSLSASVGDRVTITCRASQDISNFLNWYQQKPGKAPKLLIYYTSTLHSGVPSRFSGSGSGTDFTLTISSLQPEDFATYYCQQGKTLPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 447 aa, synthetic construct
QVQLVQSGAEVKKPGASVKVSCKASGYTFTGFWIHWVRQAPGQGLEWMGHINPGNGGTNYNEKFKRRVTMTRDKSISTAYMELSRLRSDDTAVYYCARSYSNYVRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0042984 A1
(43) Pub. Date: Feb. 6, 2025

(54) PROTEIN FORMULATIONS AND USES THEREOF

(71) Applicant: CSL Innovation Pty Ltd, Melbourne VIC (AU)

(72) Inventors: Mouhamad RESLAN, Melbourne VIC (AU); Nathan Aaron EDWARDS, Melbourne VIC (AU); Megan GARVEY, Melbourne VIC (AU); Daniel LA PORTA, Melbourne VIC (AU)

(73) Assignee: CSL Innovation Pty Ltd, Melbourne VIC (AU)

(21) Appl. No.: 18/715,228

(22) PCT Filed: Dec. 19, 2022
(86) PCT No.: PCT/AU2022/051531

§ 371 Date: May 31, 2024

(30) Foreign Application Priority Data
Dec. 20, 2021 (AU) 2021904136

(51) Int. Cl. C07K 16/22; A61K 47/18; A61K 47/22; A61K 47/26

(52) U.S. Cl. CPC C07K 16/22; A61K 47/183; A61K 47/22; A61K 47/26; C07K 2317/24; C07K 2317/52; C07K 2317/565; C07K 2317/76

(57) ABSTRACT

The present disclosure is directed to a liquid formulation comprising high concentration of a protein comprising an antigen binding domain that binds to VEGF-B, an organic buffer, a non-ionic surfactant and an amino acid stabilizer, wherein the pH of the formulation is 5.0-6.5. The formulation comprises a histidine buffer, a polysorbate 80 surfactant and the amino acid stabilizers arginine and proline.

### RELATED APPLICATION DATA

The present application claims priority from Australian Patent Application No. 2021904136 filed on 20 Dec. 2021 entitled “Protein formulations and uses thereof” The entire contents of which is hereby incorporated by reference.

### SEQUENCE LISTING

The present application is filed together with a Sequence Listing in electronic form. The entire contents of the Sequence Listing are hereby incorporated by reference.

### FIELD

The present disclosure relates to protein formulations and uses thereof. In particular, the present disclosure relates to formulations comprising a protein comprising an antigen binding domain that binds to or specifically binds to vascular endothelial growth factor B (VEGF-B).

### BACKGROUND

The VEGF family of growth factors incorporates five ligands (VEGF-A, VEGF-B, VEGF-C, VEGF-D, and placenta growth factor (PIGF)) that can bind differentially to three receptor tyrosine kinases (VEGFR-1, -2, and -3) and the semaphorin receptors (neuropilin 1 and 2). The VEGF family of growth factors are involved in normal and pathological angiogenesis and lymphangiogenisis. VEGF-A binds to VEGFR-1, VEGFR-2, NP-1, and NP-2; VEGF-B binds to VEGFR-1 and NP-1. VEGF-C and VEGF-D are mainly involved in lymphangiogenesis and bind to VEGFR-2, VEGFR-3 and NP-2.

Although antibodies and inhibitors against VEGF-B exist, there are an increasing number of challenges in formulation development for drug manufacturers. For example, there are numerous challenges associated with formulating high concentration antibody formulations (e.g., ≥50 mg/mL protein) suitable for subcutaneous administration. Formulations for subcutaneous administration typically require higher concentrations of product so as to achieve smaller injection volumes, yet increasing protein concentration often negatively impacts protein aggregation and degradation, solubility, stability, and viscosity. In addition to changes in intrinsic protein properties, manufacturing and supply chain challenges also exist including, difficulties with processing and storage to ensure that the formulated protein remains stable for long periods of time (e.g., greater than three months) and at higher temperatures (e.g., room temperature). Other challenges include optimising the rheological and syringeability properties of the final formulation. For example, viscous solutions typically require a higher injection force to administer, therefore a prolonged injection time may also be required contributing to patient pain and discomfort.

Various solutions to manufacturing high concentration antibody formulations include lyophilised formulations for reconstitution, bufferless formulations and the addition of high concentrations of salt or other additives to reduce aggregation and/or the viscosity of the formulation. However, these formulations are not effective for all antibodies. For example, the use of excessive amounts of such excipients, may lead to hypertonic preparations or changes in ionic strength of the formulation and related protein aggregation issues.

Thus, there is a need for formulations comprising protein therapeutics that bind to VEGF-B which are stable and suitable for administration to a subject for treating a disease or condition.

### SUMMARY

The present disclosure is based on the identification of a pharmaceutical formulation for a protein comprising an antigen binding domain that binds to or specifically binds to VEGF-B.

The inventors found that they can produce liquid formulations comprising high concentrations of a protein comprising an antigen binding domain that binds to VEGF-B, which remained stable, soluble, and had a viscosity suitable for injection. The formulation of the present disclosure comprises an organic acid buffer, a non-ionic surfactant, and at least one amino acid stabiliser. Notably, in producing the formulation of the present disclosure the inventors found that additional salts and/or stabilising agents were not required.

The present disclosure thus provides a liquid pharmaceutical formulation comprising a protein comprising an antigen binding domain that binds to or specifically binds to VEGF-B, an organic acid buffer, a non-ionic surfactant and at least one amino acid stabiliser, wherein the formulation has a pH of 5.0 to 6.0.

In one example, the protein is present in the formulation at a concentration of at least 50 mg/mL. In one example, the protein is present in the formulation at a concentration of at least 60 mg/mL. In one example, the protein is present in the formulation at a concentration of at least 70 mg/mL. In one example, the protein is present in the formulation at a concentration of at least 80 mg/mL. In one example, the protein is present in the formulation at a concentration of at least 90 mg/mL. In one example, the protein is present in the formulation at a concentration of at least 100 mg/mL. In one example, the protein is present in the formulation at a concentration of at least 110 mg/mL. In one example, the protein is present in the formulation at a concentration of at least 120 mg/mL. In one example, the protein is present in the formulation at a concentration of at least 130 mg/mL. In one example, the protein is present in the formulation at a concentration of at least 140 mg/mL. In one example, the protein is present in the formulation at a concentration of at least 150 mg/mL. In one example, the protein is present in the formulation at a concentration of at least 160 mg/mL. In one example, the protein is present in the formulation at a concentration of at least 170 mg/mL. In one example, the protein is present in the formulation at a concentration of at least 180 mg/mL.

In one example, the protein is present in the formulation at a concentration of at least 50 mg/mL, at least 100 mg/mL, or at least 130 mg/mL, or at least 150 mg/mL, or at least 180 mg/mL.

In one example, the protein is present in the formulation at a concentration in the range of 50 to 220 mg/mL. In one example, the protein is present in the formulation at a concentration in the range of 80 to 220 mg/mL. In one example, the protein is present in the formulation at a concentration of 140 to 220 mg/mL. In one example, the protein is present in the formulation at a concentration of about 150 mg/mL. In one example, the protein is present in the formulation at a concentration of about 160 mg/mL. In one example, the protein is present in the formulation at a concentration of about 170 mg/mL. In one example, the protein is present in the formulation at a concentration of about 180 mg/mL. In one example, the protein is present in the formulation at a concentration of about 190 mg/mL. In one example, the protein is present in the formulation at a concentration of about 200 mg/mL. In one example, the protein is present in the formulation at a concentration of about 220 mg/mL.

In one example, the protein binds to or specifically binds to VEGF-B and inhibits VEGF-B signaling. For example, the protein specifically inhibits VEGF-B signaling. This does not mean that a method of the present disclosure does not encompass inhibiting signaling of multiple VEGF proteins, only that the protein that inhibits VEGF-B signaling is specific to VEGF-B, e.g., is not a general inhibitor of VEGF proteins. This term also does not exclude, e.g., a bispecific antibody or protein comprising binding domains thereof, which can specifically inhibit VEGF-B signaling with one (or more) binding domains and can specifically inhibit signaling of another protein with another binding domain.

In one example, the protein binds to or specifically binds to VEGF-B and neutralizes VEGF-B signaling.

In one example, the protein comprises an antigen binding domain of an antibody. For instance, in some examples, the protein comprises at least a heavy chain variable region (V_{H}) and a light chain variable region (V_{L}), wherein the V_{H} and V_{L} bind to form a Fv comprising an antigen binding domain. In some examples, the protein comprises a Fv. In some examples, the protein comprises:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3; or
(x) an antibody.

In some examples, the protein is selected from the group consisting of:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3; and
(xi) an antibody.

In one example, the protein is an antibody. Exemplary antibodies are full-length and/or naked antibodies.

In one example, the protein is recombinant, chimeric, CDR grafted, humanized, synhumanized, primatized, deimmunized or human.

In one example, the protein comprises an antibody variable region that competitively inhibits the binding of antibody 2H10 to VEGF-B, wherein the antibody 2H10 comprises a heavy chain variable region (V_{H}) comprising a sequence set forth in SEQ ID NO: 3 and a light chain variable region (V_{L}) comprising a sequence set forth in SEQ ID NO: 4.

In one example, the protein comprises a heavy chain variable region (V_{H}) comprising a sequence set forth in SEQ ID NO: 3 and a light chain variable region (V_{L}) comprising a sequence set forth in SEQ ID NO: 4.

In one example, the protein comprises a humanized variable region of antibody 2H10. For example, the protein comprises a variable region comprising the complementarity determining regions (CDRs) of the V_{H} and/or the V_{L} of antibody 2H10.

In one example, the protein comprises a variable region comprising a V_{H} comprising three complementarity determining regions (CDRs) of a V_{H} comprising an amino acid sequence set forth in SEQ ID NO: 3 and a V_{L} comprising three CDRs of a V_{L} comprising an amino acid sequence set forth in SEQ ID NO: 4.

In one example, the protein comprises:
(i) a V_{H} comprising:
   (a) a CDR1 comprising a sequence set forth in amino acids 25-34 of SEQ ID NO: 3;
   (b) a CDR2 comprising a sequence set forth in amino acids 49-65 of SEQ ID NO: 3; and
   (c) a CDR3 comprising a sequence set forth in amino acids 98-108 of SEQ ID NO: 3; and/or
(ii) a V_{L} comprising:
   (a) a CDR1 comprising a sequence set forth in amino acids 23-33 of SEQ ID NO: 4;
   (b) a CDR2 comprising a sequence set forth in amino acids 49-55 of SEQ ID NO: 4; and
   (c) a CDR3 comprising a sequence set forth in amino acids 88-96 of SEQ ID NO: 4.

In one example, the protein comprises a V_{H} and a V_{L}, the V_{H} and V_{L} being humanized variable regions of antibody 2H10. For example, the protein comprises:
(i) a V_{H} comprising:
   (a) a CDR1 comprising a sequence set forth in amino acids 25-34 of SEQ ID NO: 3;
   (b) a CDR2 comprising a sequence set forth in amino acids 49-65 of SEQ ID NO: 3; and
   (c) a CDR3 comprising a sequence set forth in amino acids 98-108 of SEQ ID NO: 3; and
(ii) a V_{L} comprising:
   (a) a CDR1 comprising a sequence set forth in amino acids 23-33 of SEQ ID NO: 4;
   (b) a CDR2 comprising a sequence set forth in amino acids 49-55 of SEQ ID NO: 4; and
   (c) a CDR3 comprising a sequence set forth in amino acids 88-96 of SEQ ID NO: 4.

In one example, the variable region or V_{H} in any of the foregoing paragraphs comprises a sequence set forth in SEQ ID NO: 5.

In one example, the variable region or V_{L} in any of the foregoing paragraphs comprises a sequence set forth in SEQ ID NO: 6.

In one example, the protein is an antibody comprising a V_{H} comprising a sequence set forth in SEQ ID NO: 5 and a V_{L} comprising a sequence set forth in SEQ ID NO: 6.

In one example, the protein is an antibody comprising a light chain comprising a sequence set forth in SEQ ID NO: 47 and a heavy chain comprising a sequence set forth in SEQ ID NO: 48.

In one example, the protein or antibody is any form of the protein or antibody encoded by a nucleic acid encoding any of the foregoing proteins or antibodies.

In one example, the protein or antibody comprises:
(i) a V_{L} comprising:
   (a) a CDR1 comprising a sequence encoded by a nucleic acid comprising SEQ ID NO: 11 or comprising an amino acid sequence of SEQ ID NO: 17;
   (b) a CDR2 comprising a sequence encoded by a nucleic acid comprising SEQ ID NO: 12 or comprising an amino acid sequence of SEQ ID NO: 18; and
   (c) a CDR3 comprising a sequence encoded by a nucleic acid comprising SEQ ID NO: 13 or comprising an amino acid sequence of SEQ ID NO: 19; and/or
(ii) a V_{H} comprising:
   (a) a CDR1 comprising a sequence encoded by a nucleic acid comprising SEQ ID NO: 14 or comprising an amino acid sequence of SEQ ID NO: 20;
   (b) a CDR2 comprising a sequence encoded by a nucleic acid comprising SEQ ID NO: 15 or comprising an amino acid sequence of SEQ ID NO: 21; and
   (c) a CDR3 comprising a sequence encoded by a nucleic acid comprising SEQ ID NO: 16 or comprising an amino acid sequence of SEQ ID NO: 22.

In one example, the protein or antibody comprises:
(i) a V_{L} comprising:
   (a) a CDR1 comprising a sequence encoded by a nucleic acid comprising SEQ ID NO: 23 or comprising an amino acid sequence of SEQ ID NO: 29;
   (b) a CDR2 comprising a sequence encoded by a nucleic acid comprising SEQ ID NO: 24 or comprising an amino acid sequence of SEQ ID NO: 30; and
   (c) a CDR3 comprising a sequence encoded by a nucleic acid comprising SEQ ID NO: 25 or comprising an amino acid sequence of SEQ ID NO: 31; and/or
(ii) a V_{H} comprising:
   (a) a CDR1 comprising a sequence encoded by a nucleic acid comprising SEQ ID NO: 26 or comprising an amino acid sequence of SEQ ID NO: 32;
   (b) a CDR2 comprising a sequence encoded by a nucleic acid comprising SEQ ID NO: 27 or comprising an amino acid sequence of SEQ ID NO: 33; and
   (c) a CDR3 comprising a sequence encoded by a nucleic acid comprising SEQ ID NO: 28 or comprising an amino acid sequence of SEQ ID NO: 34.

In one example, the protein or antibody comprises:
(i) a V_{L} comprising:
   (a) a CDR1 comprising a sequence encoded by a nucleic acid comprising SEQ ID NO: 35 or comprising an amino acid sequence of SEQ ID NO: 41;
   (b) a CDR2 comprising a sequence encoded by a nucleic acid comprising SEQ ID NO: 36 or comprising an amino acid sequence of SEQ ID NO: 42; and
   (c) a CDR3 comprising a sequence encoded by a nucleic acid comprising SEQ ID NO: 37 or comprising an amino acid sequence of SEQ ID NO: 43; and/or
(ii) a V_{H} comprising:
   (a) a CDR1 comprising a sequence encoded by a nucleic acid comprising SEQ ID NO: 38 or comprising an amino acid sequence of SEQ ID NO: 44;
   (b) a CDR2 comprising a sequence encoded by a nucleic acid comprising SEQ ID NO: 39 or comprising an amino acid sequence of SEQ ID NO: 45; and
   (c) a CDR3 comprising a sequence encoded by a nucleic acid comprising SEQ ID NO: 40 or comprising an amino acid sequence of SEQ ID NO: 46

In one example, the protein or antibody is produced using a vector comprising a nucleic acid encoding a light chain of SEQ ID NO: 47 and a nucleic acid encoding a heavy chain of SEQ ID NO: 48.

In one example, the protein is a monoclonal antibody.

In one example, the antibody is an IgG antibody. For example, the antibody is an IgG₁, or an IgG₂, or an IgG₃, or an IgG₄ antibody.

In one example, the antibody is an IgG₄ antibody.

In one example, the antibody is a monoclonal IgG₄ antibody.

In one example, the protein comprises an Fc region. For example, the Fc region is a human IgG₁ Fc region or a human IgG₄ Fc region or a stabilised human IgG₄ Fc region. For example, the Fc region is a human IgG₄ Fc region. In one example, the antibody Fc region is modified to prevent dimerisation, (e.g., as discussed herein).

In one example, the antibody or antigen binding fragment thereof comprises an IgG₄ constant region.

In one example, the IgG₄ constant region is a stabilised IgG₄ constant region. For example, the IgG₄ constant region comprises a stabilised hinge region. For example, the stabilised IgG₄ constant regions comprise a proline at position 241 of the hinge region according to the system of Kabat (Kabat et al., Sequences of Proteins of Immunological Interest Washington DC United States Department of Health and Human Services, 1987 and/or 1991).

The present disclosure provides a pharmaceutical formulation comprising the protein as described herein. For example, the composition comprises a protein comprising an antibody variable region or a V_{H} or a V_{L} or an antibody as described herein. In one example, the composition additionally comprises one or more variants of the protein or antibody. For example, that comprises a variant missing an encoded C-terminal lysine residue, a deamidated variant and/or a glycosylated variant and/or a variant comprising a pyroglutamate, e.g., at the N-terminus of a protein and/or a variant lacking a N-terminal residue, e.g., a N-terminal glutamine in an antibody or V region and/or a variant comprising all or part of a secretion signal. Deamidated variants of encoded asparagine residues may result in isoaspartic, and aspartic acid isoforms being generated or even a succinamide involving an adjacent amino acid residue. Deamidated variants of encoded glutamine residues may result in glutamic acid. Compositions comprising a heterogeneous mixture of such sequences and variants are intended to be included when reference is made to a particular amino acid sequence.

In one example, the VEGF-B is mammalian VEGF-B, e.g., human VEGF-B.

In one example, the organic acid buffer is selected from the group consisting of a histidine buffer, a glutamate buffer, a succinate buffer and a citrate buffer. In one example, the organic acid buffer is selected from the group consisting of a histidine buffer and a glutamate buffer.

In one example, the organic acid buffer is an amino acid buffer. For example, the amino acid buffer is selected from the group consisting of a histidine buffer and a glutamate buffer.

In one example, the organic acid buffer is a histidine buffer. Suitable histidine buffers for use in the present disclosure will be apparent to the skilled person and included, for example, histidine chloride, histidine acetate, histidine phosphate and histidine sulfate. In one example, the histidine buffer is L-histidine.

In one example, the organic acid buffer is a glutamate buffer. Suitable glutamate buffers for use in the present disclosure will be apparent to the skilled person and include, for example, monosodium glutamate.

It will be apparent to the skilled person that buffers suitable for use in the present disclosure will provide sufficient buffer capacity to maintain the desired pH over the range of conditions to which it will be exposed during formulation and storage of the product. In one example, the formulation of the present disclosure has a pH of about 5.0 to about 6.0. In some examples, the formulation has a pH of about 5.2 to 5.9, or a pH of about 5.2 to 5.8, or a pH of about 5.3 to 5.8, or a pH of about 5.4 to about 5.9, or a pH of about 5.5 to about 5.9. In one example, the formulation has a pH of about 5.5, or about 5.6, or about 5.7, or about 5.8, or about 5.9, or about 6.0. In one example, the formulation has a pH of about 5.4 to 5.6. For example, the formulation has a pH of about 5.4. In one example, the formulation has a pH of about 5.5.

In one example, the organic acid buffer is a histidine buffer and the formulation has a pH of about 5.2 to about 5.8. In one example, the organic acid buffer is a histidine buffer and the formulation has a pH of about 5.3 to about 5.8. For example, the organic acid buffer is a histidine buffer and the formulation has a pH of about 5.3 to about 5.7. In one example, the organic acid buffer is a histidine buffer and the formulation has a pH of about 5.4 to about 5.6. For example, the organic acid buffer is a histidine buffer and the formulation has a pH of 5.4. For example, the organic acid buffer is a histidine buffer and the formulation has a pH of 5.5.

In one example, the concentration of the organic acid buffer in the pharmaceutical formulation of the present disclosure is between about 2 mM and 120 mM. In one example, the organic acid buffer is present at a concentration of a least 2 mM. For example, the organic acid buffer is present at a concentration of between about 2 mM and about 10 mM. For example, the organic acid buffer is present at a concentration of about 2 mM, or about 3 mM, or about 4 mM, or about 5 mM, or about 6 mM, or about 7 mM, or about 8 mM, or about 9 mM, or about 10 mM. In one example, the organic acid buffer is present at a concentration of at least about 10 mM. For example, the organic acid buffer is present at a concentration of between about 10 mM and about 30 mM. For example, the organic acid buffer is present at a concentration of about 10 mM, or about 12 mM, or about 14 mM, or about 16 mM, or about 18 mM, or about 20 mM, or about 25 mM, or about 30 mM. In one example, the organic acid buffer is present at a concentration of between about 12 mM and about 25 mM. For example, the organic acid buffer is present at a concentration of about 20 mM. For example, the organic acid buffer is present at a concentration of between about 10 mM and about 60 mM. For example, the organic acid buffer is present at a concentration of about 10 mM, or about 15 mM, or about 20 mM, or about 25 mM, or about 30 mM, or about 35 mM, or about 40 mM, or about 45 mM, or about 50 mM, or about 55 mM, or about 60 mM. In one example, the organic acid buffer is present at a concentration of about 20 mM.

In one example, the organic acid buffer is present in the formulation at a concentration of 10 to 30 mM.

In one example, the organic acid buffer is histidine and is present at a concentration of about 12 mM to about 25 mM. In one example, the organic buffer is histidine and is present at a concentration of about 20 mM.

In one example, the non-ionic surfactant is selected from the group consisting of polyoxyethylensorbitan fatty acid esters (e.g., polysorbate 20 and polysorbate 80), polyethylene-polypropylene copolymers, polyethylene-polypropylene glycols, polyoxyethylene-stearates, polyoxyethylene alkyl ethers, e.g. polyoxyethylene monolauryl ether, alkylphenylpolyoxyethylene ethers (Triton-X), polyoxyethylene-polyoxypropylene copolymer (Poloxamer, Pluronic), sodium dodecyl sulphate (SDS). For example, the non-ionic surfactant is selected form the group consisting of polyoxyethylensorbitan fatty acid esters and polyoxyethylene-polyoxypropylene copolymers.

In one example, the non-ionic surfactant is selected from the group consisting of polysorbate 20, polysorbate 80 and poloxamer 188.

In one example, the non-ionic surfactant is polysorbate 80.

In one example, the concentration of the non-ionic surfactant in the pharmaceutical formulation of the present disclosure is between about 0.01% (w/v) and about 1.00% (w/v). In one example, the non-ionic surfactant is present at a concentration of at least about 0.01% (w/v) or at least about 0.02% (w/v). For example, the non-ionic surfactant is present at a concentration of between about 0.01% (w/v) and about 0.10% (w/v). For example, the non-ionic surfactant is present at a concentration of about 0.01% (w/v), or about 0.02% (w/v), or about 0.03% (w/v), or about 0.04% (w/v), or about 0.05% (w/v), or about 0.06% (w/v), or about 0.07% (w/v), or about 0.08% (w/v), or about 0.09% (w/v), or about 0.10% (w/v). In one example, the non-ionic surfactant is present at a concentration of about 0.01% (w/v) or about 0.05% (w/v). In one example, the non-ionic surfactant is present at a concentration of about 0.02% (w/v) or about 0.05% (w/v).

In one example, the non-ionic surfactant is present in the formulation at a concentration of 0.02% (w/v) to 0.05 (w/v). In one example, the non-ionic surfactant is present at a concentration of about 0.02% (w/v) or about 0.04% (w/v). For example, the non-ionic surfactant is present at a concentration of about 0.02% (w/v). For example, the non-ionic surfactant is present at a concentration of about 0.03% (w/v).

In one example, the non-ionic surfactant is polysorbate 80 and is present at a concentration of between about 0.01% (w/v) and about 0.05% (w/v). In one example, the non-ionic surfactant is polysorbate 80 and is present at a concentration of between about 0.02% (w/v) and about 0.05% (w/v). For example, the non-ionic surfactant is polysorbate 80 and is present at a concentration of between about 0.02% (w/v) and about 0.04% (w/v). In one example, the non-ionic surfactant is polysorbate 80 and is present at a concentration of about 0.02% (w/v). In one example, the non-ionic surfactant is polysorbate 80 and is present at a concentration of about 0.03% (w/v).

In one example, the pharmaceutical formulation comprises at least one amino acid stabiliser selected from the group consisting of proline, arginine, glycine, alanine, valine, leucine, isoleucine, methionine, threonine, phenylalanine, tyrosine, serine, cysteine, histidine, tryptophan, aspartic acid, glutamic acid, lysine, ornithine and asparagine. For example, the amino acid stabiliser is selected from the group consisting of proline, arginine, salts thereof and a combination thereof. In one example, the amino acid stabiliser is a salt form of an amino acid discussed herein.

In one example, the at least one amino acid stabiliser includes proline and/or arginine.

In one example, the at least one amino acid stabiliser includes proline. In one example, the at least one amino acid stabiliser includes L-proline.

In one example, the at least one amino acid stabiliser includes arginine. In one example, the at least one amino acid stabiliser includes L-arginine. In one example, the at least one amino acid stabiliser includes L-arginine monohydrochloride.

In one example, the formulation comprises proline and arginine. For example, the formulation comprises L-proline and L-arginine or L-arginine monohydrochloride.

Advantageously, proline has a significant effect on thermal and aggregation stability (i.e., reduced propensity towards aggregation) compared to arginine and meglumine.

In one example, the concentration of the amino acid stabiliser in the pharmaceutical formulation of the present disclosure is between about 25 mM and about 250 mM. In one example, the amino acid stabiliser is present at a concentration of between about 50 mM and about 150 mM. For example, the amino acid stabiliser is present at a concentration of about 50 mM, or about 60 mM, or about 70 mM, or about 80 mM, or about 90 mM, or about 100 mM, or about 110 mM, or about 120 mM, or about 130 mM, or about 140 mM, or about 150 mM. In another example, the amino acid stabiliser is present at a concentration of between about 75 mM and about 125 mM. In another example, the amino acid stabiliser is present at a concentration of between about 90 mM and about 110 mM. For example, the amino acid stabiliser is present at a concentration of about 100 mM. In some examples, the formulation comprises two or more amino acid stabilisers, each present at concentration provided above.

Discussion of the foregoing concentrations also relates to a salt form of the amino acid stabiliser and the concentration recited herein is the concentration of the salt form of the amino acid rather the concentration of the amino acid per se.

In one example, the formulation comprises proline at a concentration of between 50 mM and 150 mM or between 75 mM and 125 mM or between 90 and 110 mM, for example at a concentration of about 100 mM. In some examples, the concentration of proline is less than 140 mM or less than 130 mM or less than 120 mM.

In one example, the at least one amino acid stabiliser includes proline, wherein proline is present in the formulation at a concentration of 50 mM to 150 mM In one example, the formulation comprises arginine at a concentration of between 50 mM and 150 mM, or between 75 mM and 125 mM, or between 90 and 110 mM, for example at a concentration of about 100 mM. In some examples, the concentration of arginine is less than 150 mM or less than 140 mM or less than 130 mM or less than 120 mM. In one example, the arginine is a salt form of arginine, e.g., arginine monohydrochloride and the concentration recited herein is the concentration of the salt form of arginine rather the concentration of arginine per se.

In one example, the at least one amino acid stabiliser includes arginine, wherein arginine is present in the formulation at a concentration of 50 mM to 150 mM.

In one example, the formulation comprises proline at a concentration of between 50 mM and 150 mM and arginine at a concentration of between 50 mM and 150 mM. For example, the formulation comprises about 100 mM L-proline and about 100 mM L-arginine.

In some examples, the formulation comprises a histidine buffer, proline and polysorbate 80. In some examples, the formulation further comprises arginine. In some examples, the formulation does not comprise any amino acid other than histidine, proline and arginine.

In one example, the formulation does not comprise a salt. In some examples, the formulation lacks a tonicifying amount of a salt. In some examples, the formulation does not comprise a metal salt. In some examples, the formulation does not comprise, for example, sodium chloride, calcium chloride and/or potassium chloride. Discussion of the foregoing salt does not relate to a salt form of an amino acid, or other component, in the formulation disclosed herein.

In one example, the formulation does not comprise a polyol. In one example, the formulation does not comprise a sugar, a sugar alcohol or a saccharic acid.

In one example, the formulation has a dynamic (i.e., absolute) viscosity of less than about 30 mPa*s at 20° C. In one example, the formulation has a dynamic (i.e., absolute) viscosity of less than about 20 mPa*s at 20° C. In one example, the formulation has a dynamic viscosity of less than about 15 mPa*s at 20° C. In one example, the formulation has a dynamic viscosity of less than about 10 mPa*s at 20° C. In one example, the formulation has a dynamic viscosity of between about 5 mPa*s and about mPa*s at 20° C. For example, the formulation has a dynamic viscosity of about 8 mPa*s at 20° C.

In one example, the formulation has a dynamic viscosity of less than 20 mPa*s at 20° C., less than 10 mPa*s at 20° C., or less than 8 mPa*s at 20° C. For example, the formulation has a dynamic viscosity of about 7.6 mPa*s at 20° C.

Methods of assessing viscosity will be apparent to the skilled person and/or are described herein. For example, viscosity may be assessed by use of a microviscometer, such as a rolling-ball viscometer. A rolling-ball viscometer measures the rolling time of a ball through transparent and opaque liquids according to Hoppler's falling ball principle. An example of a rolling-ball viscometer is the Anton Par Lovis 2000 M Microviscometer.

In one example, the osmolality of the formulation is between about 150 mOsm/kg and about 550 mOsm/kg. For example, the osmolality of the formulation is about 150 mOsm/kg, or about 175 mOsm/kg, or about 200 mOsm/kg, or about 225 mOsm/kg, or about 250 mOsm/kg, or about 275 mOsm/kg, or about 300 mOsm/kg, or about 325 mOsm/kg, or about 350 mOsm/kg, or about 375 mOsm/kg, or about 400 mOsm/kg, or about 425 mOsm/kg, or about 450 mOsm/kg, or about 475 mOsm/kg, or about 500 m Osm/kg, or about 550 mOsm/kg. In one example, the osmolality of the formulation is between about 250 mOsm/kg and about 450 mOsm/kg. For example, the osmolality of the formulation is about 250 mOsm/kg, or about 260 mOsm/kg, or about 270 mOsm/kg, or about 280 mOsm/kg, or about 290 mOsm/kg, or about 300 mOsm/kg, or about 310 mOsm/kg, or about 320 mOsm/kg, or about 330 mOsm/kg, or about 340 mOsm/kg, or about 350 mOsm/kg, or about 360 mOsm/kg, or about 370 mOsm/kg, or about 380 mOsm/kg, or about 390 mOsm/kg, or about 400 mOsm/kg. In one example, the osmolality is between about 350 mOsm/kg and about 390 mOsm/kg. In one example, the osmolality is between about 350 mOsm/kg and about 380 mOsm/kg. In one example, the osmolality is between about 360 mOsm/kg and about 390 mOsm/kg. For example, the osmolality is between about 360 mOsm/kg and about 380 mOsm/kg.

In some examples, the formulation is a stable formulation. The stability of the formulation may be assessed by any means known in the art. For example, the stability of the formulation may be assessed by measuring total high molecular weight species (HMWS) and/or monomer content. Methods for assessing accumulation of HMWS and monomer content of the formulation will be apparent to the skilled person and/or described herein. In one example, the percent HMWS of the protein in the formulation is determined by size-exclusion chromatography (e.g., SEC or SE-HPLC).

Any reference herein to a formulation being a stable formulation also includes a formulation comprising a protein of the disclosure (i.e., a protein comprising an antigen binding domain that binds to or specifically binds to vascular endothelial growth factor B (VEGF-B)).

In another example, the formation of HMWS of the protein is assessed using dynamic light scattering (DLS). For example, the fluctuation of light intensity using a digital correlator (e.g., Malvern Zetasizer software) is measured and the Z-average hydrodynamic diameter and polydispersity index (using e.g., a cumulants analysis) are determined.

In some examples, the formulation comprises no more than 5% high molecular weight species (HMWS). In some examples, the formulation comprises no more than 5% HMWS, as determined by size exclusion chromatography (SEC). In some examples, the formulation comprises no more than 5% HMWS, as determined by size exclusion high performance liquid chromatography (SE-HPLC).

In some examples, the formulation of the present disclosure comprises at least 90% monomer protein and/or less than (i.e., no more than) 10% HMWS and/or low molecular weight species (LMWS, i.e., degraded or fragmented). In one example, the formulation comprises at least 95% monomer protein and/or less than (i.e., no more than) 5% HMWS and/or LMWS.

In one example, the formulation comprises no more than about 10% HMWS. For example, the formulation comprises no more than about 10%, or no more than about 9%, or no more than about 8%, or no more than about 7%, or no more than about 6%, or no more than about 5%, or no more than about 4%, or no more than about 3%, or no more than about 2%, or no more than about 1% HMWS.

In some examples, the formulation comprises no more than 5% high molecular weight species (HMWS) after storage for a period of at least 1 month, or at least 3 months, or at least 6 months, or at least 12 months, or at least 18 months, or at least 24 months at a temperature in the range of 2° C. to 35° C. For example, the formulation comprises no more than 5% high molecular weight species (HMWS) after storage for a period of at least 1 month, or at least 3 months, or at least 6 months at a temperature in the range of 2° C. to 35° C. In one example, the formulation comprises no more than 5% high molecular weight species (HMWS) after storage for a period of at least 1 month, or at least 3 months, or at least 6 months, or at least 9 months, or at least 12 months at a temperature in the range of 2° C. to 35° C. For example, the formulation comprises no more than 5% HMWS after storage for a period of at least 1 month at a temperature in the range of 2° C. to 35° C. In another example, the formulation comprises no more than 5% HMWS after storage for a period of at least 3 months at a temperature in the range of 2° C. to 35° C. In a further example, the formulation comprises no more than 5% HMWS after storage for a period of at least 3 months at a temperature in the range of 2° C. to 35° C. In one example, the formulation comprises no more than 5% HMWS after storage for a period of at least 6 months at a temperature in the range of 2° C. to 35° C. In another example, the formulation comprises no more than 5% HMWS after storage for a period of at least 9 months at a temperature in the range of 2° C. to 35° C. In a further example, the formulation comprises no more than 5% HMWS after storage for a period of at least 12 months at a temperature in the range of 2° C. to 35° C.

In one example, the formulation comprises no more than 5% HMWS after storage for a period of at least 1 month at a temperature of about 35° C. For example, the formulation comprises no more than 3% HMWS after storage for a period of at least 1 month at a temperature of about 35° C. In one example, the formulation comprises no more than 5% HMWS after storage for a period of at least 1 month at a temperature of about 25° C. For example, the formulation comprises no more than 2% HMWS after storage for a period of at least 1 month at a temperature of about 25° C. In one example, the formulation comprises no more than 5% HMWS after storage for a period of at least 1 month at a temperature of about 5° C. For example, the formulation comprises no more than 1% HMWS after storage for a period of at least 1 month at a temperature of about 5° C.

In one example, the formulation comprises no more than 5% HMWS after storage for a period of at least 3 months at a temperature of about 35° C. In one example, the formulation comprises no more than 5% HMWS after storage for a period of at least 3 months at a temperature of about 25° C. In one example, the formulation comprises no more than 3% HMWS after storage for a period of at least 3 months at a temperature of about 5° C.

In one example, the formulation comprises no more than 5% HMWS after storage for a period of at least 6 months at a temperature of about 25° C. In one example, the formulation comprises no more than 3% HMWS after storage for a period of at least 6 months at a temperature of about 5° C.

In one example, the formulation comprises no more than 5% HMWS after storage for a period of at least 9 months at a temperature of about 25° C. In one example, the formulation comprises no more than 4% HMWS after storage for a period of at least 9 months at a temperature of about 5° C.

In one example, the formulation comprises no more than 5% HMWS after storage for a period of at least 12 months at a temperature of about 25° C. In one example, the formulation comprises no more than 4% HMWS after storage for a period of at least 12 months at a temperature of about 5° C.

In some examples, at least 90% of the protein in the formulation is a monomer. In some example, at least 95% of the protein in the formulation is a monomer. In some examples, at least 95% of the protein in the formulation is a monomer, as determined by SEC. In some examples, at least 95% of the protein in the formulation is a monomer, as determined by SE-HPLC.

In some examples, at least 90% of the protein in the formulation is a monomer. In some examples, at least 95% of the protein in the formulation is a monomer. In some examples, at least 95%, or at least 96%, or at least 97%, or at least 98%, or at least 99% of the protein in the formulation is a monomer.

In some examples, at least 90% of the protein in the formulation is a monomer after storage for a period of at least 1 month, or at least 3 months, or at least 6 months, or at least 12 months, or at least 18 months, or at least 24 months at a temperature in the range of 2° C. to 35° C. For example, at least 90% of the protein in the formulation is a monomer after storage for a period of at least 1 month at a temperature in the range of 2° C. to 35° C. In another example, at least 90% of the protein in the formulation is a monomer after storage for a period of at least 3 months at a temperature in the range of 2° C. to 35° C. In a further example, at least 90% of the protein in the formulation is a monomer after storage for a period of at least 6 months at a temperature in the range of 2° C. to 35° C. In another example, at least 90% of the protein in the formulation is a monomer after storage for a period of at least 12 months at a temperature in the range of 2° C. to 35° C. In another example, at least 90% of the protein in the formulation is a monomer after storage for a period of at least 18 months at a temperature in the range of 2° C. to 35° C. In another example, at least 90% of the protein in the formulation is a monomer after storage for a period of at least 24 month at a temperature in the range of 2° C. to 35° C.

In some examples, at least 95% of the protein in the formulation is a monomer after storage for a period of at least 1 month, or at least 3 months, or at least 6 months at a temperature in the range of 2° C. to 30° C. In another example, at least 95% of the protein in the formulation is a monomer after storage for a period of at least 1 month, or at least 3 months, or at least 6 months, or at least 9 months, or at least 12 months at a temperature in the range of 2° C. to 30° C.

In one example, at least 95% of the protein in the formulation is a monomer after storage for a period of at least 1 month at a temperature of about 35° C. In one example, at least 95% of the protein in the formulation is a monomer after storage for a period of at least 1 month at a temperature of about 25° C. In one example, at least 97% of the protein in the formulation is a monomer after storage for a period of at least 1 month at a temperature of about 5° C.

In one example, at least 94% of the protein in the formulation is a monomer after storage for a period of at least 3 months at a temperature of about 35° C. In one example, at least 95% of the protein in the formulation is a monomer after storage for a period of at least 3 months at a temperature of about 25° C. In one example, at least 97% of the protein in the formulation is a monomer after storage for a period of at least 3 months at a temperature of about 5° C.

In one example, at least 95% of the protein in the formulation is a monomer after storage for a period of at least 6 months at a temperature of about 25° C. In one example, at least 98% of the protein in the formulation is a monomer after storage for a period of at least 6 months at a temperature of about 5° C.

In one example, at least 95% of the protein in the formulation is a monomer after storage for a period of at least 9 months at a temperature of about 25° C. In one example, at least 97% of the protein in the formulation is a monomer after storage for a period of at least 9 months at a temperature of about 5° C.

In one example, at least 95% of the protein in the formulation is a monomer after storage for a period of at least 12 months at a temperature of about 25° C. In one example, at least 97% of the protein in the formulation is a monomer after storage for a period of at least 12 months at a temperature of about 5° C.

In some examples, the formulation comprises no more than 10% LMWS. In some examples, the formulation comprises no more than 10% LMWS, as determined by capillary electrophoresis with sodium dodecylsulfate (CE-SDS) under non-reducing conditions.

In some examples, the formulation comprises no more than 10%, or no more than 9%, or no more than 8%, or no more than 7%, or no more than 6%, or no more than 5% LMWS.

In some examples, the formulation comprises no more than 5% LMWS. In some examples, the formulation comprises no more than 5% LMWS, as determined by capillary electrophoresis with sodium dodecylsulfate (CE-SDS) under non-reducing conditions.

In some examples, the formulation comprises no more than 10% LMWS after storage for a period of at least 1 month, or at least 3 months, or at least 6 months, or at least 12 months, or at least 18 months, or at least 24 month at a temperature in the range of 2° C. to 35° C. In one example, the formulation comprises no more than 10% LMWS after storage for a period of at least 1 month at a temperature in the range of 2° C. to 35° C. In one example, the formulation comprises no more than 10% LMWS after storage for a period of at least 3 months at a temperature in the range of 2° C. to 35° C. In one example, the formulation comprises no more than 10% LMWS after storage for a period of at least 6 months at a temperature in the range of 2° C. to 35° C. In one example, the formulation comprises no more than 10% LMWS after storage for a period of at least 12 months at a temperature in the range of 2° C. to 35° C. In one example, the formulation comprises no more than 10% LMWS after storage for a period of at least 18 months at a temperature in the range of 2° C. to 35° C. In one example, the formulation comprises no more than 10% LMWS after storage for a period of at least 24 months at a temperature in the range of 2° C. to 35° C.

In some examples, the formulation comprises no more than 5%, or no more than 4%, or no more than 3%, or no more than 2% LMWS.

In some examples, the formulation comprises no more than 5% LMWS after storage for a period of at least 1 month, or at least 3 months, or at least 6 months at a temperature in the range of 2° C. to 30° C. In one example, the formulation comprises no more than 5% LMWS after storage for a period of at least 1 month, or at least 3 months, or at least 6 months, or at least 9 months, or at least 12 months at a temperature in the range of 2° C. to 30° C.

In one example, the formulation comprises no more than 5% LMWS after storage for a period of at least 1 month at a temperature of about 35° C. For example, the formulation comprises no more than 3% LMWS after storage for a period of at least 1 month at a temperature of about 35° C. In one example, the formulation comprises no more than 5% LMWS after storage for a period of at least 1 month at a temperature of about 25° C. For example, the formulation comprises no more than 2% LMWS after storage for a period of at least 1 month at a temperature of about 25° C. In one example, the formulation comprises no more than 5% LMWS after storage for a period of at least 1 month at a temperature of about 5° C. For example, the formulation comprises no more than 2% LMWS after storage for a period of at least 1 month at a temperature of about 5° C.

In one example, the formulation comprises no more than 6% LMWS after storage for a period of at least 3 months at a temperature of about 35° C. In one example, the formulation comprises no more than 5% LMWS after storage for a period of at least 3 months at a temperature of about 25° C. In one example, the formulation comprises no more than 2% LMWS after storage for a period of at least 3 months at a temperature of about 5° C.

In one example, the formulation comprises no more than 5% LMWS after storage for a period of at least 6 months at a temperature of about 25° C. In one example, the formulation comprises no more than 2% LMWS after storage for a period of at least 6 months at a temperature of about 5° C.

In one example, the formulation comprises no more than 5% LMWS after storage for a period of at least 9 months at a temperature of about 25° C. In one example, the formulation comprises no more than 3% LMWS after storage for a period of at least 9 months at a temperature of about 5° C.

In one example, the formulation comprises no more than 6% LMWS after storage for a period of at least 12 months at a temperature of about 25° C. In one example, the formulation comprises no more than 3% LMWS after storage for a period of at least 12 months at a temperature of about 5° C.

In some examples of the formulation of the disclosure, one or more or all of the following apply:
a) the formulation comprises no more than 5% high molecular weight species (HMWS), as determined by size exclusion high performance liquid chromatography (SE-HPLC);
b) at least 95% of the protein in the formulation is a monomer, as determined by SE-HPLC; and
c) the formulation comprises no more than 5% low molecular weight species (LMWS), as determined by capillary electrophoresis with sodium dodecylsulfate (CE-SDS) under non-reducing conditions.

In some examples, the amount of HMWS, monomer or LMWS described above is determined after storage for a period of at least 1 month, or at least 3 months or at least 6 months at a temperature in the range of 2° C. to 35° C. In one example, the amount of HMWS, monomer, or LMWS is determined after storage for a period of at least 1 month, or at least 3 months or at least 6 months at a temperature in the range of 2° C. to 8° C. For example, at a temperature of 5° C. In another example, the amount of HMWS, monomer or LMWS is determined after storage for a period of at least 1 month, or at least 3 months or at least 6 months at a temperature in the range 22° C. to 28° C. For example, at a temperature of 25° C. In one example, the amount of HMWS, monomer, or LMWS is determined after storage for a period of at least 1 month, or at least 3 months or at least 6 months at a temperature in the range of 33° C. to 37° C. For example, at a temperature of 35° C.

In some examples, the amount of HMWS, monomer or LMWS described above is determined after storage for a period of at least 1 month, or at least 3 months or at least 6 months, or at least 9 months, or at least 12 months at a temperature in the range of 2° C. to 35° C. In one example, the amount of HMWS, monomer, or LMWS is determined after storage for a period of at least 1 month, or at least 3 months or at least 6 months, or at least 9 months, or at least 12 months at a temperature in the range of 2° C. to 8° C. For example, at a temperature of 5° C. In another example, the amount of HMWS, monomer or LMWS is determined after storage for a period of at least 1 month, or at least 3 months or at least 6 months, or at least 9 months, or at least 12 months at a temperature in the range 22° C. to 28° C. For example, at a temperature of 25° C. In one example, the amount of HMWS, monomer, or LMWS is determined after storage for a period of at least 1 month, or at least 3 months or at least 6 months, or at least 9 months, or at least 12 months at a temperature in the range of 33° C. to 37° C. For example, at a temperature of 35° C.

In some examples, after storage of the formulation for a period of at least 6 months at a temperature of about 25° C., one or more or all of the following apply:
a) the formulation comprises no more than 5% high molecular weight species (HMWS), as determined by size exclusion high performance liquid chromatography (SE-HPLC);
b) at least 95% of the protein in the formulation is a monomer, as determined by SE-HPLC; and
c) the formulation comprises no more than 5% low molecular weight species (LMWS), as determined by capillary electrophoresis with sodium dodecylsulfate (CE-SDS) under non-reducing conditions.

In some examples, after storage of the formulation for a period of at least 12 months at a temperature of about 25° C., one or more or all of the following apply:
a) the formulation comprises no more than 5% high molecular weight species (HMWS), as determined by size exclusion high performance liquid chromatography (SE-HPLC);
b) at least 95% of the protein in the formulation is a monomer, as determined by SE-HPLC; and
c) the formulation comprises no more than 5% low molecular weight species (LMWS), as determined by capillary electrophoresis with sodium dodecylsulfate (CE-SDS) under non-reducing conditions.

In some examples, after storage of the formulation for a period of at least 12 months at a temperature of about 25° C., one or more or all of the following apply:
a) the formulation comprises no more than 5% high molecular weight species (HMWS), as determined by size exclusion high performance liquid chromatography (SE-HPLC);
b) at least 95% of the protein in the formulation is a monomer, as determined by SE-HPLC; and
c) the formulation comprises no more than 6% low molecular weight species (LMWS), as determined by capillary electrophoresis with sodium dodecylsulfate (CE-SDS) under non-reducing conditions.

In some examples, after storage of the formulation for a period of at least 6 months at a temperature of about 5° C., one or more or all of the following apply:
a) the formulation comprises no more than 3% high molecular weight species (HMWS), as determined by size exclusion high performance liquid chromatography (SE-HPLC);
b) at least 97% of the protein in the formulation is a monomer, as determined by SE-HPLC; and
c) the formulation comprises no more than 2% low molecular weight species (LMWS), as determined by capillary electrophoresis with sodium dodecylsulfate (CE-SDS) under non-reducing conditions.

In some examples, after storage of the formulation for a period of at least 12 months at a temperature of about 5° C., one or more or all of the following apply:
a) the formulation comprises no more than 3% high molecular weight species (HMWS), as determined by size exclusion high performance liquid chromatography (SE-HPLC);
b) at least 97% of the protein in the formulation is a monomer, as determined by SE-HPLC; and
c) the formulation comprises no more than 2% low molecular weight species (LMWS), as determined by capillary electrophoresis with sodium dodecylsulfate (CE-SDS) under non-reducing conditions.

In some examples, the formulation is an aqueous formulation. In one example, the formulation is suitable for subcutaneous administration. In some examples, the formulation has a volume in the range of 0.2 mL to 10 mL. In some examples, the formulation has a volume in the range of 0.5 mL to 5 mL. In some examples, the formulation has a volume in the range of 1 mL to 3 mL. In some examples, the formulation has a volume of about 1 mL, or about 2 mL, or about 3 mL, or about 4 mL, or about 5 mL.

In one example, the formulation has not previously been lyophilised. In one example, the formulation is not a reconstituted formulation.

The present disclosure provides a liquid pharmaceutical formulation comprising a protein comprising an antigen binding domain that binds to or specifically binds to VEGF-B, an organic acid buffer comprising histidine, a surfactant selected from the group consisting of polysorbate 20, polysorbate 80 and poloxamer 188, and at least one amino acid stabiliser including proline and/or arginine, wherein the formulation has a pH of 5.0 to 6.0.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.0 to 6.0. For example, the formulation has a pH of 5.1 to 5.9. In one example, the formulation has a pH of 5.2 to 5.8. In another example, the formulation has a pH of 5.3 to 5.7. In a further example, the formulation has a pH of 5.4 to 5.6.

In some examples, the formulation has a pH of 5.3 to 5.7 and comprises 5 mM to 50 mM histidine buffer, 0.02% to 0.05% (w/v) polysorbate 80, 50 mM to 150 mM proline and 50 mM to 150 mM arginine.

In some examples, the formulation has a pH of 5.4 to 5.6 and comprises 5 mM to 50 mM histidine buffer, 0.02% to 0.05% (w/v) polysorbate 80, 50 mM to 150 mM proline and 50 mM to 150 mM arginine.

In some examples, the formulation has a pH of 5.3 to 5.7 and comprises 5 mM to 50 mM histidine buffer, 0.02% to 0.04% (w/v) polysorbate 80, 50 mM to 150 mM proline and 50 mM to 150 mM arginine.

In some examples, the formulation has a pH of 5.4 to 5.6 and comprises 5 mM to 50 mM histidine buffer, 0.02% to 0.04% (w/v) polysorbate 80, 50 mM to 150 mM proline and 50 mM to 150 mM arginine.

In some examples, the formulation has a pH of 5.3 to 5.7 and comprises 10 mM to 30 mM histidine buffer, 0.02% to 0.04% (w/v) polysorbate 80, 80 mM to 120 mM proline and 80 mM to 120 mM arginine.

In some examples, the formulation has a pH of 5.4 to 5.6 and comprises 10 mM to 30 mM histidine buffer, 0.02% to 0.04% (w/v) polysorbate 80, 80 mM to 120 mM proline and 80 mM to 120 mM arginine.

In some examples, the formulation has a pH of 5.3 to 5.7 and comprises 15 mM to 25 mM histidine buffer, 0.02% to 0.04% (w/v) polysorbate 80, 90 mM to 110 mM proline and 90 mM to 110 mM arginine.

In some examples, the formulation has a pH of 5.4 to 5.6 and comprises 15 mM to 25 mM histidine buffer, 0.02% to 0.04% (w/v) polysorbate 80, 90 mM to 110 mM proline and 90 mM to 110 mM arginine.

In some examples, the present disclosure provides a liquid pharmaceutical formulation comprising a protein comprising an antigen binding domain that binds to or specifically binds to VEGF-B, 15 mM to 25 mM histidine buffer, 0.02% to 0.04% (w/v) polysorbate 80, 90 mM to 110 mM proline and 90 mM to 110 mM arginine, wherein the formulation has a pH of 5.3 to 5.7.

In some examples, the present disclosure provides a liquid pharmaceutical formulation comprising a protein comprising an antigen binding domain that binds to or specifically binds to VEGF-B, 15 mM to 25 mM histidine buffer, 0.02% to 0.04% (w/v) polysorbate 80, 90 mM to 110 mM proline and 90 mM to 110 mM arginine, wherein the formulation has a pH of 5.4 to 5.6.

In some examples, the formulation has a pH of 5.3 to 5.7 and comprises about 20 mM histidine buffer, about 0.03% (w/v) polysorbate 80, about 100 mM proline and about 100 mM arginine.

In some examples, the formulation has a pH of 5.4 to 5.6 and comprises about 20 mM histidine buffer, about 0.03% (w/v) polysorbate 80, about 100 mM proline and about 100 mM arginine.

In some examples, the formulation has a pH of 5.5 and comprises 20 mM histidine buffer, 0.03% (w/v) polysorbate 80, 100 mM proline and 100 mM arginine.

The present disclosure provides a liquid pharmaceutical formulation comprising about 50 mg/ml of a protein comprising an antigen binding domain that binds to or specifically binds to VEGF-B, 20 mM histidine buffer, 0.03% (w/v) polysorbate 80, 100 mM proline and 100 mM arginine, and has a pH of 5.5.

The present disclosure provides a liquid pharmaceutical formulation comprising about 100 mg/ml of a protein comprising an antigen binding domain that binds to or specifically binds to VEGF-B, 20 mM histidine buffer, 0.03% (w/v) polysorbate 80, 100 mM proline and 100 mM arginine, and has a pH of 5.5.

The present disclosure provides a liquid pharmaceutical formulation comprising about 160 mg/ml of a protein comprising an antigen binding domain that binds to or specifically binds to VEGF-B, 20 mM histidine buffer, 0.03% (w/v) polysorbate 80, 100 mM proline and 100 mM arginine, and has a pH of 5.5.

The present disclosure provides a liquid pharmaceutical formulation comprising about 180 mg/ml of a protein comprising an antigen binding domain that binds to or specifically binds to VEGF-B, 20 mM histidine buffer, 0.02% (w/v) polysorbate 80, 100 mM proline and 100 mM arginine, and has a pH of 5.5.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.0 to 6.0, and wherein the protein comprises a V_{H} comprising three CDRs of a V_{H} comprising an amino acid sequence set forth in SEQ ID NO: 3 and a V_{L} comprising three CDRs of a V_{L} comprising an amino acid sequence set forth in SEQ ID NO: 4.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.3 to 5.7, and wherein the protein comprises a V_{H} comprising three CDRs of a V_{H} comprising an amino acid sequence set forth in SEQ ID NO: 3 and a V_{L} comprising three CDRs of a V_{L} comprising an amino acid sequence set forth in SEQ ID NO: 4.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.4 to 5.6, and wherein the protein comprises a V_{H} comprising three CDRs of a V_{H} comprising an amino acid sequence set forth in SEQ ID NO: 3 and a V_{L} comprising three CDRs of a V_{L} comprising an amino acid sequence set forth in SEQ ID NO: 4.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.5, and wherein the protein comprises a V_{H} comprising three CDRs of a V_{H} comprising an amino acid sequence set forth in SEQ ID NO: 3 and a V_{L} comprising three CDRs of a V_{L} comprising an amino acid sequence set forth in SEQ ID NO: 4.

The present disclosure also provides a liquid pharmaceutical formulation comprising a protein comprising an antigen binding domain that binds to or specifically binds to VEGF-B, 10 mM to 30 mM histidine buffer, 0.02% to 0.05% polysorbate 80, 50 mM to 150 mM proline and 50 mM to 150 mM arginine, wherein the formulation has a pH of 5.0 to 6.0, and wherein the protein comprises a V_{H} comprising three CDRs of a V_{H} comprising an amino acid sequence set forth in SEQ ID NO: 3 and a V_{L} comprising three CDRs of a V_{L} comprising an amino acid sequence set forth in SEQ ID NO: 4.

The present disclosure also provides a liquid pharmaceutical formulation comprising a protein comprising an antigen binding domain that binds to or specifically binds to VEGF-B, 10 mM to 30 mM histidine buffer, 0.02% to 0.05% polysorbate 80, 50 mM to 150 mM proline and 50 mM to 150 mM arginine, wherein the formulation has a pH of 5.3 to 5.7, and wherein the protein comprises a V_{H} comprising three CDRs of a V_{H} comprising an amino acid sequence set forth in SEQ ID NO: 3 and a V_{L} comprising three CDRs of a V_{L} comprising an amino acid sequence set forth in SEQ ID NO: 4.

The present disclosure also provides a liquid pharmaceutical formulation comprising a protein comprising an antigen binding domain that binds to or specifically binds to VEGF-B, 10 mM to 30 mM histidine buffer, 0.02% to 0.05% polysorbate 80, 50 mM to 150 mM proline and 50 mM to 150 mM arginine, wherein the formulation has a pH of 5.4 to 5.6, and wherein the protein comprises a V_{H} comprising three CDRs of a V_{H} comprising an amino acid sequence set forth in SEQ ID NO: 3 and a V_{L} comprising three CDRs of a V_{L} comprising an amino acid sequence set forth in SEQ ID NO: 4.

The present disclosure also provides a liquid pharmaceutical formulation comprising a protein comprising an antigen binding domain that binds to or specifically binds to VEGF-B, 15 mM to 25 mM histidine buffer, 0.02% to 0.04% polysorbate 80, 90 mM to 110 mM proline and 90 mM to 110 mM arginine, wherein the formulation has a pH of 5.0 to 6.0, and wherein the protein comprises a V_{H} comprising three CDRs of a V_{H} comprising an amino acid sequence set forth in SEQ ID NO: 3 and a V_{L} comprising three CDRs of a V_{L} comprising an amino acid sequence set forth in SEQ ID NO: 4.

The present disclosure also provides a liquid pharmaceutical formulation comprising a protein comprising an antigen binding domain that binds to or specifically binds to VEGF-B, 15 mM to 25 mM histidine buffer, 0.02% to 0.04% polysorbate 80, 90 mM to 110 mM proline and 90 mM to 110 mM arginine, wherein the formulation has a pH of 5.3 to 5.7, and wherein the protein comprises a V_{H} comprising three CDRs of a V_{H} comprising an amino acid sequence set forth in SEQ ID NO: 3 and a V_{L} comprising three CDRs of a V_{L} comprising an amino acid sequence set forth in SEQ ID NO: 4.

The present disclosure also provides a liquid pharmaceutical formulation comprising a protein comprising an antigen binding domain that binds to or specifically binds to VEGF-B, 15 mM to 25 mM histidine buffer, 0.02% to 0.04% polysorbate 80, 90 mM to 110 mM proline and 90 mM to 110 mM arginine, wherein the formulation has a pH of 5.4 to 5.6, and wherein the protein comprises a V_{H} comprising three CDRs of a V_{H} comprising an amino acid sequence set forth in SEQ ID NO: 3 and a V_{L} comprising three CDRs of a V_{L} comprising an amino acid sequence set forth in SEQ ID NO: 4.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.0 to 6.0, and wherein the protein comprises a V_{H} comprising an amino acid sequence set forth in SEQ ID NO: 5 and a V_{L} comprising an amino acid sequence set forth in SEQ ID NO: 6.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.3 to 5.7, and wherein the protein comprises a V_{H} comprising an amino acid sequence set forth in SEQ ID NO: 5 and a V_{L} comprising an amino acid sequence set forth in SEQ ID NO: 6.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.4 to 5.6, and wherein the protein comprises a V_{H} comprising an amino acid sequence set forth in SEQ ID NO: 5 and a V_{L} comprising an amino acid sequence set forth in SEQ ID NO: 6.

The present disclosure also provides a liquid pharmaceutical formulation comprising a protein comprising an antigen binding domain that binds to or specifically binds to VEGF-B, 10 mM to 30 mM histidine buffer, 0.02% to 0.05% polysorbate 80, 50 mM to 150 mM proline and 50 mM to 150 mM arginine, wherein the formulation has a pH of 5.0 to 6.0, and wherein the protein comprises a V_{H} comprising an amino acid sequence set forth in SEQ ID NO: 5 and a V_{L} comprising an amino acid sequence set forth in SEQ ID NO: 6.

The present disclosure also provides a liquid pharmaceutical formulation comprising a protein comprising an antigen binding domain that binds to or specifically binds to VEGF-B, 10 mM to 30 mM histidine buffer, 0.02% to 0.05% polysorbate 80, 50 mM to 150 mM proline and 50 mM to 150 mM arginine, wherein the formulation has a pH of 5.3 to 5.7, and wherein the protein comprises a V_{H} comprising an amino acid sequence set forth in SEQ ID NO: 5 and a V_{L} comprising an amino acid sequence set forth in SEQ ID NO: 6.

The present disclosure also provides a liquid pharmaceutical formulation comprising a protein comprising an antigen binding domain that binds to or specifically binds to VEGF-B, 10 mM to 30 mM histidine buffer, 0.02% to 0.05% polysorbate 80, 50 mM to 150 mM proline and 50 mM to 150 mM arginine, wherein the formulation has a pH of 5.4 to 5.6, and wherein the protein comprises a V_{H} comprising an amino acid sequence set forth in SEQ ID NO: 5 and a V_{L} comprising an amino acid sequence set forth in SEQ ID NO: 6.

The present disclosure also provides a liquid pharmaceutical formulation comprising a protein comprising an antigen binding domain that binds to or specifically binds to VEGF-B, 15 mM to 25 mM histidine buffer, 0.02% to 0.04% polysorbate 80, 90 mM to 110 mM proline and 90 mM to 110 mM arginine, wherein the formulation has a pH of 5.0 to 6.0, and wherein the protein comprises a V_{H} comprising an amino acid sequence set forth in SEQ ID NO: 5 and a V_{L} comprising an amino acid sequence set forth in SEQ ID NO: 6.

The present disclosure also provides a liquid pharmaceutical formulation comprising a protein comprising an antigen binding domain that binds to or specifically binds to VEGF-B, 15 mM to 25 mM histidine buffer, 0.02% to 0.04% polysorbate 80, 90 mM to 110 mM proline and 90 mM to 110 mM arginine, wherein the formulation has a pH of 5.3 to 5.7, and wherein the protein comprises a V_{H} comprising an amino acid sequence set forth in SEQ ID NO: 5 and a V_{L} comprising an amino acid sequence set forth in SEQ ID NO: 6.

The present disclosure also provides a liquid pharmaceutical formulation comprising a protein comprising an antigen binding domain that binds to or specifically binds to VEGF-B, 15 mM to 25 mM histidine buffer, 0.02% to 0.04% polysorbate 80, 90 mM to 110 mM proline and 90 mM to 110 mM arginine, wherein the formulation has a pH of 5.4 to 5.6, and wherein the protein comprises a V_{H} comprising an amino acid sequence set forth in SEQ ID NO: 5 and a V_{L} comprising an amino acid sequence set forth in SEQ ID NO: 6.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.0 to 6.0, and wherein the protein comprises a light chain comprising an amino acid sequence set forth in SEQ ID NO: 47 and a heavy chain comprising an amino acid sequence set forth in SEQ ID NO: 48.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.3 to 5.7, and wherein the protein comprises a light chain comprising an amino acid sequence set forth in SEQ ID NO: 47 and a heavy chain comprising an amino acid sequence set forth in SEQ ID NO: 48.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.4 to 5.6, and wherein the protein comprises a light chain comprising an amino acid sequence set forth in SEQ ID NO: 47 and a heavy chain comprising an amino acid sequence set forth in SEQ ID NO: 48.

The present disclosure also provides a liquid pharmaceutical formulation comprising a protein comprising an antigen binding domain that binds to or specifically binds to VEGF-B, 10 mM to 30 mM histidine buffer, 0.02% to 0.05% polysorbate 80, 50 mM to 150 mM proline and 50 mM to 150 mM arginine, wherein the formulation has a pH of 5.0 to 6.0, and wherein the protein comprises a light chain comprising an amino acid sequence set forth in SEQ ID NO: 47 and a heavy chain comprising an amino acid sequence set forth in SEQ ID NO: 48.

The present disclosure also provides a liquid pharmaceutical formulation comprising a protein comprising an antigen binding domain that binds to or specifically binds to VEGF-B, 10 mM to 30 mM histidine buffer, 0.02% to 0.05% polysorbate 80, 50 mM to 150 mM proline and 50 mM to 150 mM arginine, wherein the formulation has a pH of 5.3 to 5.7, and wherein the protein comprises a light chain comprising an amino acid sequence set forth in SEQ ID NO: 47 and a heavy chain comprising an amino acid sequence set forth in SEQ ID NO: 48.

The present disclosure also provides a liquid pharmaceutical formulation comprising a protein comprising an antigen binding domain that binds to or specifically binds to VEGF-B, 10 mM to 30 mM histidine buffer, 0.02% to 0.05% polysorbate 80, 50 mM to 150 mM proline and 50 mM to 150 mM arginine, wherein the formulation has a pH of 5.4 to 5.6, and wherein the protein comprises a light chain comprising an amino acid sequence set forth in SEQ ID NO: 47 and a heavy chain comprising an amino acid sequence set forth in SEQ ID NO: 48.

The present disclosure also provides a liquid pharmaceutical formulation comprising a protein comprising an antigen binding domain that binds to or specifically binds to VEGF-B, 15 mM to 25 mM histidine buffer, 0.02% to 0.04% polysorbate 80, 90 mM to 110 mM proline and 90 mM to 110 mM arginine, wherein the formulation has a pH of 5.0 to 6.0, and wherein the protein comprises a light chain comprising an amino acid sequence set forth in SEQ ID NO: 47 and a heavy chain comprising an amino acid sequence set forth in SEQ ID NO: 48.

The present disclosure also provides a liquid pharmaceutical formulation comprising a protein comprising an antigen binding domain that binds to or specifically binds to VEGF-B, 15 mM to 25 mM histidine buffer, 0.02% to 0.04% polysorbate 80, 90 mM to 110 mM proline and 90 mM to 110 mM arginine, wherein the formulation has a pH of 5.3 to 5.7, and wherein the protein comprises a light chain comprising an amino acid sequence set forth in SEQ ID NO: 47 and a heavy chain comprising an amino acid sequence set forth in SEQ ID NO: 48.

The present disclosure also provides a liquid pharmaceutical formulation comprising a protein comprising an antigen binding domain that binds to or specifically binds to VEGF-B, 15 mM to 25 mM histidine buffer, 0.02% to 0.04% polysorbate 80, 90 mM to 110 mM proline and 90 mM to 110 mM arginine, wherein the formulation has a pH of 5.4 to 5.6, and wherein the protein comprises a light chain comprising an amino acid sequence set forth in SEQ ID NO: 47 and a heavy chain comprising an amino acid sequence set forth in SEQ ID NO: 48.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.0 to 6.0, and wherein the protein comprises:
a) a V_{H} comprising a CDR1 comprising an amino acid sequence set forth in SEQ ID NO: 20, a CDR2 comprising an amino acid sequence set forth in SEQ ID NO: 21 and a CDR3 comprising an amino acid sequence set forth in SEQ ID NO: 22; and
b) a V_{L} comprising a CDR1 comprising an amino acid sequence set forth in SEQ ID NO: 17, a CDR2 comprising an amino acid sequence set forth in SEQ ID NO: 18 and a CDR3 comprising an amino acid sequence set forth in SEQ ID NO: 19.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.3 to 5.7, and wherein the protein comprises:
a) a V_{H} comprising a CDR1 comprising an amino acid sequence set forth in SEQ ID NO: 20, a CDR2 comprising an amino acid sequence set forth in SEQ ID NO: 21 and a CDR3 comprising an amino acid sequence set forth in SEQ ID NO: 22; and
b) a V_{L} comprising a CDR1 comprising an amino acid sequence set forth in SEQ ID NO: 17, a CDR2 comprising an amino acid sequence set forth in SEQ ID NO: 18 and a CDR3 comprising an amino acid sequence set forth in SEQ ID NO: 19.

The present disclosure also provides a liquid pharmaceutical formulation comprising a protein comprising an antigen binding domain that binds to or specifically binds to VEGF-B, a histidine buffer, polysorbate 80, proline and arginine, wherein the formulation has a pH of 5.4 to 5.6, and wherein the protein comprises:
a) a V_{H} comprising a CDR1 comprising an amino acid sequence set forth in SEQ ID NO: 20, a CDR2 comprising an amino acid sequence set forth in SEQ ID NO: 21 and a CDR3 comprising an amino acid sequence set forth in SEQ ID NO: 22; and
b) a V_{L} comprising a CDR1 comprising an amino acid sequence set forth in SEQ ID NO: 17, a CDR2 comprising an amino acid sequence set forth in SEQ ID NO: 18 and a CDR3 comprising an amino acid sequence set forth in SEQ ID NO: 19.

The present disclosure also provides a liquid pharmaceutical formulation comprising a protein comprising an antigen binding domain that binds to or specifically binds to VEGF-B, 10 mM to 30 mM histidine buffer, 0.01% to 0.05% polysorbate 80, 50 mM to 150 mM proline and 50 mM to 150 mM arginine, wherein the formulation has a pH of 5.0 to 6.0, and wherein the protein comprises:
a) a V_{H} comprising a CDR1 comprising an amino acid sequence set forth in SEQ ID NO: 20, a CDR2 comprising an amino acid sequence set forth in SEQ ID NO: 21 and a CDR3 comprising an amino acid sequence set forth in SEQ ID NO: 22; and
b) a V_{L} comprising a CDR1 comprising an amino acid sequence set forth in SEQ ID NO: 17, a CDR2 comprising an amino acid sequence set forth in SEQ ID NO: 18 and a CDR3 comprising an amino acid sequence set forth in SEQ ID NO: 19.

The present disclosure also provides a liquid pharmaceutical formulation comprising a protein comprising an antigen binding domain that binds to or specifically binds to VEGF-B, 10 mM to 30 mM histidine buffer, 0.01% to 0.05% polysorbate 80, 50 mM to 150 mM proline and 50 mM to 150 mM arginine, wherein the formulation has a pH of 5.3 to 5.7, and wherein the protein comprises:
a) a V_{H} comprising a CDR1 comprising an amino acid sequence set forth in SEQ ID NO: 20, a CDR2 comprising an amino acid sequence set forth in SEQ ID NO: 21 and a CDR3 comprising an amino acid sequence set forth in SEQ ID NO: 22; and
b) a V_{L} comprising a CDR1 comprising an amino acid sequence set forth in SEQ ID NO: 17, a CDR2 comprising an amino acid sequence set forth in SEQ ID NO: 18 and a CDR3 comprising an amino acid sequence set forth in SEQ ID NO: 19.

The present disclosure also provides a liquid pharmaceutical formulation comprising a protein comprising an antigen binding domain that binds to or specifically binds to VEGF-B, 10 mM to 30 mM histidine buffer, 0.01% to 0.05% polysorbate 80, 50 mM to 150 mM proline and 50 mM to 150 mM arginine, wherein the formulation has a pH of 5.4 to 5.6, and wherein the protein comprises:
a) a V_{H} comprising a CDR1 comprising an amino acid sequence set forth in SEQ ID NO: 20, a CDR2 comprising an amino acid sequence set forth in SEQ ID NO: 21 and a CDR3 comprising an amino acid sequence set forth in SEQ ID NO: 22; and
b) a V_{L} comprising a CDR1 comprising an amino acid sequence set forth in SEQ ID NO: 17, a CDR2 comprising an amino acid sequence set forth in SEQ ID NO: 18 and a CDR3 comprising an amino acid sequence set forth in SEQ ID NO: 19.

The present disclosure also provides a liquid pharmaceutical formulation comprising a protein comprising an antigen binding domain that binds to or specifically binds to VEGF-B, 15 mM to 25 mM histidine buffer, 0.01% to 0.03% polysorbate 80, 90 mM to 110 mM proline and 90 mM to 110 mM arginine, wherein the formulation has a pH of 5.0 to 6.0, and wherein the protein comprises:
a) a V_{H} comprising a CDR1 comprising an amino acid sequence set forth in SEQ ID NO: 20, a CDR2 comprising an amino acid sequence set forth in SEQ ID NO: 21 and a CDR3 comprising an amino acid sequence set forth in SEQ ID NO: 22; and
b) a V_{L} comprising a CDR1 comprising an amino acid sequence set forth in SEQ ID NO: 17, a CDR2 comprising an amino acid sequence set forth in SEQ ID NO: 18 and a CDR3 comprising an amino acid sequence set forth in SEQ ID NO: 19.

The present disclosure also provides a liquid pharmaceutical formulation comprising a protein comprising an antigen binding domain that binds to or specifically binds to VEGF-B, 15 mM to 25 mM histidine buffer, 0.01% to 0.03% polysorbate 80, 90 mM to 110 mM proline and 90 mM to 110 mM arginine, wherein the formulation has a pH of 5.3 to 5.7, and wherein the protein comprises:
a) a V_{H} comprising a CDR1 comprising an amino acid sequence set forth in SEQ ID NO: 20, a CDR2 comprising an amino acid sequence set forth in SEQ ID NO: 21 and a CDR3 comprising an amino acid sequence set forth in SEQ ID NO: 22; and
b) a V_{L} comprising a CDR1 comprising an amino acid sequence set forth in SEQ ID NO: 17, a CDR2 comprising an amino acid sequence set forth in SEQ ID NO: 18 and a CDR3 comprising an amino acid sequence set forth in SEQ ID NO: 19.

The present disclosure also provides a liquid pharmaceutical formulation comprising a protein comprising an antigen binding domain that binds to or specifically binds to VEGF-B, 15 mM to 25 mM histidine buffer, 0.01% to 0.03% polysorbate 80, 90 mM to 110 mM proline and 90 mM to 110 mM arginine, wherein the formulation has a pH of 5.4 to 5.6, and wherein the protein comprises:
a) a V_{H} comprising a CDR1 comprising an amino acid sequence set forth in SEQ ID NO: 20, a CDR2 comprising an amino acid sequence set forth in SEQ ID NO: 21 and a CDR3 comprising an amino acid sequence set forth in SEQ ID NO: 22; and
b) a V_{L} comprising a CDR1 comprising an amino acid sequence set forth in SEQ ID NO: 17, a CDR2 comprising an amino acid sequence set forth in SEQ ID NO: 18 and a CDR3 comprising an amino acid sequence set forth in SEQ ID NO: 19.

The present disclosure also provides a method of treating or preventing or delaying progression of a disease or condition in a subject, the method comprising administering the formulation described herein to the subject.

The present disclosure also provides a formulation described herein for use in treating or preventing or delaying progression of a disease or condition in a subject.

The present disclosure also provides use of the formulation described herein in the manufacture of a medicament for use in treating or preventing or delaying progression of a disease or condition in a subject.

In one example, the disease or condition is selected from the group consisting of nephropathy, a wound, a non-alcoholic fatty liver disease (NAFLD) or complication thereof, a stroke, a wasting disorder, obesity, insulin resistance, diabetes, a cardiovascular disorder, metabolic syndrome, and combinations thereof.

In one example, the disease or condition is nephropathy. For example, the nephropathy is diabetic nephropathy (i.e., nephropathy associated diabetes). For example, the subject suffers from nephropathy associated with type 1 diabetes. For example, the subject suffers from nephropathy associated with type 2 diabetes. In another example, the subject suffers from diabetes and microalbuminuria or diabetes and macroalbuminuria.

In one example, the disease or condition is a wound. The wound can be a chronic, acute or normal wound. In one example, the wound is chronic. In one example, a subject suffers from a wound that is infected and/or ischemic. In another example, the wound is a full-thickness wound. In a further example, the wound is a diabetic ulcer, e.g., a diabetic foot ulcer.

In one example, the disease or condition is a non-alcoholic fatty liver disease (NAFLD) or a complication thereof. In one example, the NAFLD is selected from the group consisting of hepatic steatosis (e.g., isolated hepatic steatosis), non-alcoholic steatohepatitis (NASH), cirrhosis, NASH-derived cirrhosis, NASH-associated cirrhosis, NASH-associated hepatic fibrosis. In one example, the NAFLD is hepatic steatosis or NASH or cirrhosis or hepatic fibrosis. In one example, the hepatic steatosis is severe hepatic steatosis. In one example, the complication of the NAFLD is hepatocellular carcinoma.

In one example, the disease or condition is a stroke. For example, the stroke is an ischemic stroke. For example, the stroke is a cerebral ischemic stroke.

In one example, the disease or condition is a wasting disorder. In one example, the wasting disorder is selected from the group consisting of cachexia, unintended body weight loss, fat wasting and anorexia. For example, the cachexia is selected from the group consisting of cancer cachexia, chronic kidney disease cachexia and diabetic cachexia.

In one example, the disease or condition is obesity.

In one example, the disease or condition is insulin resistance.

In one example, the disease or condition is diabetes. For example, type I or type II diabetes.

In one example, the disease or condition is a cardiovascular disorder. In one example, the cardiovascular disease is elevated blood pressure, atherosclerosis, heart failure or a cardiovascular event such as acute coronary syndrome, myocardial infarction, myocardial ischemia, chronic stable angina pectoris, unstable angina pectoris, angioplasty, stroke, transient ischemic attack, claudication(s), or vascular occlusion(s).

In one example, the disease or condition is a metabolic syndrome.

In one example, the formulation of the disclosure is administered subcutaneously to the subject in need thereof. In another example, the formulation of the disclosure is administered intravenously to the subject in need thereof.

In one example, the formulation of the disclosure is self-administered.

In one example, the formulation of the disclosure is self-administered subcutaneously.

In one example, the formulation of the disclosure is provided in a pre-filled syringe.

In one example, the formulation of the disclosure is self-administered subcutaneously, with a pre-filled syringe.

In one example, the formulation of the disclosure is provided in a vial. For example, the formulation is provided in a glass vial.

In one example of any method described herein, the subject is a mammal, for example a primate such as a human.

Methods of treatment described herein can additionally comprise administering a further compound to reduce, treat or prevent or delay the progression of the disease or condition described herein.

The present disclosure also provides a kit for use in treating or preventing or delaying progression of a disease or condition in a subject, the kit comprising:
(a) at least one pharmaceutical formulation described herein;
(b) instructions for using the kit in treating or preventing or delaying the disease or condition in the subject; and
(c) optionally, at least one further therapeutically active compound or drug.

In some examples, the formulation is present in a vial, a prefilled syringe or an autoinjector device.

The present disclosure also provides a vial comprising the pharmaceutical formulation described herein.

The present disclosure also provides a prefilled syringe comprising the pharmaceutical formulation described herein.

The present disclosure also provides an autoinjector device comprising the pharmaceutical formulation described herein.

Exemplary effects of the pharmaceutical formulation of the present disclosure are described herein and are to be taken to apply mutatis mutandis to the examples of the disclosure set out in the previous paragraphs.

### BRIEF DESCRIPTION OF THE DRAWINGS

FIG. 1 is a graphical representation showing HMWS species by SE-HPLC of 180 mg/mL CSL346 formulations containing 100 or 150 mM arginine combined with other stabiliser components over 3 months storage at 35° C.

FIG. 2 is a graphical representation showing the effect of methionine on the rate of change in HMWS in CSL346 formulations.

FIG. 3 is a graphical representation showing the effect of PS80 on sub-visible particle counts for (A) 10-25 μm and (B) >25 μm in CSL346 formulations after 4 weeks of storage at 35° C.

FIG. 4 is a graphical representation showing the effect of silicone oil on HMWS formation in CSL346 formulations.

FIG. 5 is a graphical representation showing the effect of tungsten on HMWS formation in CSL346 formulations.

### KEY TO SEQUENCE LISTING

SEQ ID NO: 1 is an amino acid sequence of a human VEGF-B₁₈₆ isoform containing a 21 amino acid N-terminal signal sequence
SEQ ID NO: 2 is an amino acid sequence of a human VEGF-B₁₆₇ isoform containing a 21 amino acid N-terminal signal sequence
SEQ ID NO: 3 is an amino acid sequence from a V_{H} of antibody 2H10.
SEQ ID NO: 4 is an amino acid sequence from a V_{L} of antibody 2H10.
SEQ ID NO: 5 is an amino acid sequence from a V_{H} of a humanized form of antibody 2H10.
SEQ ID NO: 6 is an amino acid sequence of a V_{L} of a humanized form of antibody 2H10.
SEQ ID NO: 7 is an amino acid sequence from a V_{H} of antibody 4E12.
SEQ ID NO: 8 is an amino acid sequence of a V_{L} of antibody 4E12.
SEQ ID NO: 9 is an amino acid sequence from a V_{H} of antibody 2F5.
SEQ ID NO: 10 is an amino acid sequence of a V_{L} of antibody 2F5.
SEQ ID NO: 11 is a nucleotide sequence from a V_{L} CDR1 of antibody 2H10
SEQ ID NO: 12 is a nucleotide sequence from a V_{L} CDR2 of antibody 2H10
SEQ ID NO: 13 is a nucleotide sequence from a V_{L} CDR3 of antibody 2H10
SEQ ID NO: 14 is a nucleotide sequence from a V_{H} CDR1 of antibody 2H10
SEQ ID NO: 15 is a nucleotide sequence from a V_{H} CDR2 of antibody 2H10
SEQ ID NO: 16 is a nucleotide sequence from a V_{H} CDR3 of antibody 2H10
SEQ ID NO: 17 is an amino acid sequence from a V_{L} CDR1 of antibody 2H10
SEQ ID NO: 18 is an amino acid sequence from a V_{L} CDR2 of antibody 2H10
SEQ ID NO: 19 is an amino acid sequence from a V_{L} CDR3 of antibody 2H10
SEQ ID NO: 20 is an amino acid sequence from a V_{H} CDR1 of antibody 2H10
SEQ ID NO: 21 is an amino acid sequence from a V_{H} CDR2 of antibody 2H10
SEQ ID NO: 22 is an amino acid sequence from a V_{H} CDR3 of antibody 2H10
SEQ ID NO: 23 is a nucleotide sequence from a V_{L} CDR1 of antibody 2F5
SEQ ID NO: 24 is a nucleotide sequence from a V_{L} CDR2 of antibody 2F5
SEQ ID NO: 25 is a nucleotide sequence from a V_{L} CDR3 of antibody 2F5
SEQ ID NO: 26 is a nucleotide sequence from a V_{H} CDR1 of antibody 2F5
SEQ ID NO: 27 is a nucleotide sequence from a V_{H} CDR2 of antibody 2F5
SEQ ID NO: 28 is a nucleotide sequence from a V_{H} CDR3 of antibody 2F5
SEQ ID NO: 29 is an amino acid sequence from a V_{L} CDR1 of antibody 2F5
SEQ ID NO: 30 is an amino acid sequence from a V_{L} CDR2 of antibody 2F5
SEQ ID NO: 31 is an amino acid sequence from a V_{L} CDR3 of antibody 2F5
SEQ ID NO: 32 is an amino acid sequence from a V_{H} CDR1 of antibody 2F5
SEQ ID NO: 33 is an amino acid sequence from a V_{H} CDR2 of antibody 2F5
SEQ ID NO: 34 is an amino acid sequence from a V_{H} CDR3 of antibody 2F5
SEQ ID NO: 35 is a nucleotide sequence from a V_{L} CDR1 of antibody 4E12
SEQ ID NO: 36 is a nucleotide sequence from a V_{L} CDR2 of antibody 4E12
SEQ ID NO: 37 is a nucleotide sequence from a V_{L} CDR3 of antibody 4E12
SEQ ID NO: 38 is a nucleotide sequence from a V_{H} CDR1 of antibody 4E12
SEQ ID NO: 39 is a nucleotide sequence from a V_{H} CDR2 of antibody 4E12
SEQ ID NO: 40 is a nucleotide sequence from a V_{H} CDR3 of antibody 4E12
SEQ ID NO: 41 is an amino acid sequence from a V_{L} CDR1 of antibody 4E12
SEQ ID NO: 42 is an amino acid sequence from a V_{L} CDR2 of antibody 4E12
SEQ ID NO: 43 is an amino acid sequence from a V_{L} CDR3 of antibody 4E12
SEQ ID NO: 44 is an amino acid sequence from a V_{H} CDR1 of antibody 4E12
SEQ ID NO: 45 is an amino acid sequence from a V_{H} CDR2 of antibody 4E12
SEQ ID NO: 46 is an amino acid sequence from a V_{H} CDR3 of antibody 4E12
SEQ ID NO: 47 is an amino acid sequence from a light chain of a humanized form of antibody 2H10
SEQ ID NO: 48 is an amino acid sequence from a heavy chain of a humanized form of antibody 2H10

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, TRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and LeskJ. Mol Biol. 196:901-917, 1987, Chothia et al. Nature 342, 877-883, 1989 and/or or Al-Lazikani et al., J Mol Biol 273, 927-948, 1997.

Any discussion of a protein or antibody herein will be understood to include any variants of the protein or antibody produced during manufacturing and/or storage. For example, during manufacturing or storage an antibody can be deamidated (e.g., at an asparagine or a glutamine residue) and/or have altered glycosylation and/or have a glutamine residue converted to pyroglutamine and/or have a N-terminal or C-terminal residue removed or “clipped” and/or have part or all of a signal sequence incompletely processed and, as a consequence, remain at the terminus of the antibody. It is understood that a composition comprising a particular amino acid sequence may be a heterogeneous mixture of the stated or encoded sequence and/or variants of that stated or encoded sequence.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

VEGF-B is known to exist in two major isoforms, referred to as VEGF-B₁₈₆ and VEGF-B₁₆₇. For the purposes of nomenclature only and not limitation exemplary sequences of human VEGF-B₁₈₆ is set out in NCBI Reference Sequence: NP_003368.1, in NCBI protein accession numbers NP_003368, P49765 and AAL79001 and in SEQ ID NO: 1. In the context of the present disclosure, the sequence of VEGF-B₁₈₆ can lack the 21 amino acid N-terminal signal sequence (e.g., as set out at amino acids 1 to 21 of SEQ ID NO: 1. For the purposes of nomenclature only and not limitation exemplary sequences of human VEGF-B₁₆₇ is set out in NCBI Reference Sequence: NP_001230662.1, in NCBI protein accession numbers AAL79000 and AAB06274 and in SEQ ID NO: 2. In the context of the present disclosure, the sequence of VEGF-B₁₆₇ can lack the 21 amino acid N-terminal signal sequence (e.g., as set out at amino acids 1 to 21 of SEQ ID NO: 2. Additional sequence of VEGF-B can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)). Reference to human VEGF-B may be abbreviated to hVEGF-B. In one example, reference herein to VEGF-B is to VEGF-B₁₆₇ isoform.

Reference herein to VEGF-B also encompasses the VEGF-B₁₀₋₁₀₈ peptide as described in WO2006/012688.

The term “organic acid buffer” refers to conventional buffers of organic acids and salts. Suitable organic acid buffers for use in the formulation of the present disclosure are described herein.

The term “non-ionic surfactant” as used herein refers to any detergent that has an uncharged polar head. Suitable surfactants for use in the formulation of the present disclosure are described herein.

A “stable” formulation is one in which the protein in the formulation essentially retains its physical stability and/or chemical stability and/or biological activity upon storage.

In the context of the present disclosure, the term “monomer” or “monomeric” refers to the correctly folded protein (e.g., antibody or antigen binding fragment thereof). For example, a monomer of an antibody according to the present disclosure relates to the standard tetrameric antibody comprising two identical, glycosylated heavy and light chains respectively. An “aggregate” is a non-specific association of two or more protein molecules (e.g., high molecular weight species).

As used herein, the term “amino acid stabiliser” refers to an amino acid or derivative thereof that improves or otherwise enhances the stability of the formulation.

As used herein, the term “polyol” refers to a substance having a plurality of hydroxyl groups.

The term “dynamic viscosity” or “absolute viscosity” refers to the internal resistance to flow exhibited by a fluid at a specified temperature (e.g., 20° C.), the ratio of shearing stress to rate of shear. A liquid has a dynamic viscosity of one poise if a force of 1 dyne/square centimetre causes two parallel liquid surfaces one square centimetre in area and one square centimetre apart to move past one another at a velocity of 1 cm/second. One poise equals one hundred centipoise (cP) and one centipoise equals one millipascal-second (mPa*s) in System International (SI) units.

As used herein, the term “osmolality” is a measure of the osmoles (Osm) of solute per kilogram of solvent (osmol/kg or Osm/kg).

As used herein, the term “binds” is a reference to an interaction of a protein with another molecule that is dependent upon the presence of a particular structure (e.g., an antigenic determinant or epitope) on that molecule. For example, an antibody, or antigen binding fragment thereof, recognises and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that a protein of the disclosure reacts or associates more frequently, more rapidly, with greater duration and/or with greater affinity with a particular antigen or cell expressing same than it does with alternative antigens or cells. For example, a protein binds to VEGF-B with materially greater affinity (e.g., 20 fold or 40 fold or 60 fold or 80 fold to 100 fold or 150 fold or 200 fold) than it does to other growth factor (e.g., VEGF-A) or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

As used herein, the term “neutralize” shall be taken to mean that a protein is capable of blocking, reducing or preventing VEGF-B-signaling in a cell through the VEGF-R1. Methods for determining neutralization are known in the art and/or described herein.

As used herein, the term “specifically inhibits VEGF-B signaling” will be understood to mean that the compound inhibits VEGF-B signaling and does not significantly or detectably inhibit signaling by one or more other VEGF proteins, e.g., VEGF-A, VEGF-C, VEGF-D and/or PIGF.

As used herein, the term “does not significantly inhibit” shall be understood to mean that the level of inhibition of signaling by a VEGF protein other than VEGF-B (e.g., signalling by VEGF-A, VEGF-C, VEGF-D and/or PIGF) in the presence of a compound described herein is not statistically significantly lower than in the absence of the compound described herein (e.g., in a control assay which may be conducted in the presence of an isotype control antibody).

As used herein, the term “does not detectably inhibit” shall be understood to mean that a compound as described herein inhibits signalling of a VEGF protein other than VEGF-B (e.g., signalling by VEGF-A, VEGF-C, VEGF-D and/or PIGF) by no more than 10% or 8% or 6% or 5% or 4% or 3% or 2% or 1% of the level of signalling detected in the absence of the compound described herein (e.g., in a control assay which may be conducted in the presence of an isotype control antibody).

The term “recombinant” shall be understood to mean the product of artificial genetic recombination. Accordingly, in the context of a protein comprising an antigen binding domain described herein, this term does not encompass an antibody naturally occurring within a subject's body that is the product of natural recombination that occurs during B cell maturation. However, if such an antibody is isolated, it is to be considered an isolated protein comprising an antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding domain” or “antigen binding site” shall be taken to mean a structure formed by a protein that is capable of binding or specifically binding to an antigen. The antigen binding domain need not be a series of contiguous amino acids, or even amino acids in a single polypeptide chain. For example, in a Fv produced from two different polypeptide chains the antigen binding domain is made up of a series of amino acids of a V_{L} and a V_{H} that interact with the antigen and that are generally, however not always in the one or more of the CDRs in each variable region. In some examples, an antigen binding domain is or comprises a V_{H} or a V_{L} or a Fv. In some examples, the antigen binding domain comprises one or more CDRs of an antibody.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are necessary for antigen binding. Each variable region typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196: 901-917, 1987; Chothia et al. Nature 342, 877-883, 1989; and/or Al-Lazikani et al., J Mol Biol 273: 927-948, 1997; the IMGT numbering system of Lefranc et al., Devel. And Compar. Immunol., 27: 55-77, 2003; or the AHO numbering system of Honnegher and Plukthun J. Mol. Biol., 309: 657-670, 2001. For example, according to the numbering system of Kabat, V_{H} framework regions (FRs) and CDRs are positioned as follows: residues 1-30 (FR1), 31-(CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including those discussed above. In one example, reference herein to a CDR (or a FR) is in respect of those regions according to the Kabat numbering system.

“Framework regions” (FRs) are those variable region residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)₂ fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

The term “fragment crystallisable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Exemplary heavy chain constant regions are gamma 1 (IgG₁), gamma 2 (IgG₂), gamma 3 (IgG₃) and gamma 4 (IgG₄), or hybrids thereof.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy C_{H} C_{H}1, a linker, a C_{H}2 and a C_{H}3. In a light chain, a constant region generally comprises one constant domain (a C_{L}1).

The term “stabilised IgG₄ constant region” will be understood to mean an IgG₄ constant region that has been modified to reduce Fab arm exchange or the propensity to undergo Fab arm exchange or formation of a half-antibody or a propensity to form a half antibody. “Fab arm exchange” refers to a type of protein modification for human IgG₄, in which an IgG₄ heavy chain and attached light chain (half-molecule) is swapped for a heavy-light chain pair from another IgG₄ molecule. Thus, IgG₄ molecules may acquire two distinct Fab arms recognising two distinct antigens (resulting in bispecific molecules). Fab arm exchange occurs naturally in vivo and can be induced in vitro by purified blood cells or reducing agents such as reduced glutathione.

The term “competitively inhibits” shall be understood to mean that a protein of the disclosure (or an antigen binding site thereof) reduces or prevents binding of a recited antibody or protein to VEGF-B, e.g., to hVEGF-B. This may be due to the protein (or antigen binding site) and antibody binding to the same or an overlapping epitope. It will be apparent from the foregoing that the protein need not completely inhibit binding of the antibody, rather it need only reduce binding by a statistically significant amount, for example, by at least about 10% or 20% or 30% or 40% or 50% or 60% or 70% or 80% or 90% or 95%. Preferably, the protein reduces binding of the antibody by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to VEGF-B either in the presence or absence of the protein. If less antibody binds in the presence of the protein than in the absence of the protein, the protein is considered to competitively inhibit binding of the antibody. In one example, the competitive inhibition is not due to steric hindrance.

As used herein, the terms “disease”, “disorder” or “condition” refers to a disruption of or interference with normal function, and is not to be limited to any specific condition, and will include diseases or disorders.

As used herein, the terms “treating”, “treat” or “treatment” include administering a protein described herein to thereby reduce or eliminate at least one symptom of a specified disease or condition or to slow progression of the disease or condition.

As used herein, the terms “preventing”, “prevent” or “prevention” includes providing prophylaxis with respect to occurrence or recurrence of a specified disease or condition in an individual. An individual may be predisposed to or at risk of developing the disease or disease relapse but has not yet been diagnosed with the disease or the relapse.

As used herein, a subject “at risk” of developing a disease or condition or relapse thereof or relapsing may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the term “subject” shall be taken to mean any animal including humans, for example a mammal. Exemplary subjects include but are not limited to humans and non-human primates. For example, the subject is a human.

### Proteins of the Pharmaceutical Formulation

As discussed herein, the present disclosure provides a liquid pharmaceutical formulation comprising a protein comprising an antigen binding domain that binds to or specifically binds to VEGF-B. In some examples, the protein comprises at least a V_{H} and a V_{L}, wherein the V_{H} and V_{L} bind to form a Fv comprising an antigen binding domain.

### Proteins Comprising Antigen Binding Domains

In one example, the protein comprising an antigen binding domain that binds to or specifically binds to VEGF-B comprises an antibody variable region, e.g., is an antibody or antigen binding fragment. For example, the protein is an antibody or antigen binding fragment that binds to VEGF-B and neutralizes VEGF-B signaling.

Methods for generating antibodies are known in the art and/or described in Harlow and Lane (editors) Antibodies: A Laboratory Manual, Cold Spring Harbor Laboratory, (1988). Generally, in such methods VEGF-B or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route. Methods for producing anti-VEGF-B antibodies in mice are described in WO2006/012688.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present disclosure may be a synthetic antibody. For example, the antibody is a chimeric antibody, a humanised antibody, a human antibody or a de-immunised antibody.

The antibodies or antigen binding fragments of the present disclosure may be humanised.

The term “humanised antibody” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanised antibodies also include antibodies in which one or more residues of the human protein are modified by one or more amino acid substitutions and/or one or more FR residues of the human antibody are replaced by corresponding non-human residues. Humanised antibodies may also comprise residues which are found in neither the human antibody or in the non-human antibody. Any additional regions of the antibody (e.g., Fc region) are generally human. Humanisation can be performed using a method known in the art, e.g., U.S. Pat. Nos. 5,225,539, 6,054,297, 7,566,771 or U.S. Pat. No. 5,585,089. The term “humanised antibody” also encompasses a super-humanised antibody, e.g., as described in U.S. Pat. No. 7,732,578. A similar meaning will be taken to apply to the term “humanised antigen binding fragment”.

The antibodies or antigen binding fragments thereof of the present disclosure may be human antibodies or antigen binding fragments thereof. The term “human antibody” as used herein refers to antibodies having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human antibody will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516. A similar meaning will be taken to apply to the term “human antigen binding fragment”.

The antibodies or antigen binding fragments thereof of the present disclosure may be synhumanised antibodies or antigen binding fragments thereof. The term “synhumanised antibody” refers to an antibody prepared by a method described in WO2007019620. A synhumanised antibody includes a variable region of an antibody, wherein the variable region comprises FRs from a New World primate antibody variable region and CDRs from a non-New World primate antibody variable region.

The antibody or antigen binding fragment thereof of the present disclosure may be primatised. A “primatised antibody” comprises variable region(s) from an antibody generated following immunisation of a non-human primate (e.g., a cynomolgus macaque). Optionally, the variable regions of the non-human primate antibody are linked to human constant regions to produce a primatised antibody. Exemplary methods for producing primatised antibodies are described in U.S. Pat. No. 6,113,898.

In one example an antibody or antigen binding fragment thereof of the disclosure is a chimeric antibody or fragment. The term “chimeric antibody” or “chimeric antigen binding fragment” refers to an antibody or fragment in which one or more of the variable domains is from a particular species (e.g., murine, such as mouse or rat) or belonging to a particular antibody class or subclass, while the remainder of the antibody or fragment is from another species (such as, for example, human or non-human primate) or belonging to another antibody class or subclass. In one example, a chimeric antibody comprising a V_{H} and/or a V_{L} from a non-human antibody (e.g., a murine antibody) and the remaining regions of the antibody are from a human antibody. The production of such chimeric antibodies and antigen binding fragments thereof is known in the art, and may be achieved by standard means (as described, e.g., in U.S. Pat. Nos. 6,331,415; 5,807,715; 4,816,567 and 4,816,397).

The present disclosure also contemplates a deimmunised antibody or antigen binding fragment thereof, e.g., as described in WO2000034317 and WO2004108158. De-immunised antibodies and fragments have one or more epitopes, e.g., B cell epitopes or T cell epitopes removed (i.e., mutated) to thereby reduce the likelihood that a subject will raise an immune response against the antibody or protein. For example, an antibody of the disclosure is analysed to identify one or more B or T cell epitopes and one or more amino acid residues within the epitope is mutated to thereby reduce the immunogenicity of the antibody.

In one example, the antibody variable region binds specifically to VEGF-B.

Suitable antibodies and proteins comprising variable regions thereof are known in the art and/or described herein. Exemplary anti-VEGF-B antibodies and fragments thereof are also described in WO2006/012688.

### Antibody Fragments

As described herein, a protein of the disclosure comprises an antigen binding fragment of an antibody. Exemplary antigen binding fragments for use in the present disclosure are described below.

### Single-Domain Antibodies

In some examples, an antigen binding fragment of an antibody of the disclosure is or comprises a single-domain antibody (which is used interchangeably with the term “domain antibody” or “dAb”). A single-domain antibody is a single polypeptide chain comprising all or a portion of the heavy chain variable domain of an antibody.

### Diabodies, Triabodies, Tetrabodies

In some examples, an antigen binding fragment of the disclosure is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv) Fragments

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favoured linkers for a scFv.

In one example, the linker comprises the sequence SGGGGSGGGGSGGGGS.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present disclosure encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Heavy Chain Antibodies

In some examples, an antigen binding fragment of the disclosure is or comprises a heavy chain antibody. Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR). A general description of heavy chain antibodies from camelids and the variable regions thereof and methods for their production and/or isolation and/or use is found inter alia in the following references WO 94/04678, WO 97/49805 and WO 97/49805. A general description of heavy chain antibodies from cartilaginous fish and the variable regions thereof and methods for their production and/or isolation and/or use is found inter alia in WO 2005/118629.

### Half-Antibodies

In some examples, the antigen binding fragment of the present disclosure is a half-antibody or a half-molecule. The skilled artisan will be aware that a half antibody refers to a protein comprising a single heavy chain and a single light chain. The term “half antibody” also encompasses a protein comprising an antibody light chain and an antibody heavy chain, wherein the antibody heavy chain has been mutated to prevent association with another antibody heavy chain. In one example, a half antibody forms when an antibody dissociates to form two molecules each containing a single heavy chain and a single light chain.

Methods for generating half antibodies are known in the art and exemplary methods are described herein.

In one example, the half antibody can be secreted by introducing into cells genes of the single heavy chain and single light chain that constitute the IgG of interest for expression. In one example, a constant region (e.g., an IgG₄ constant region) comprises a “key or hole” (or “knob or hole”) mutation to prevent heterodimer formation. In one example, a constant region (e.g., an IgG₄ constant region) comprises a T366W mutation (or knob). In another example, a constant region (e.g., an IgG₄ constant region) comprises a T366S, L368A and Y407V mutation (or hole). In another example, the constant region comprises T350V, T366L, K392L and T394W mutations (knob). In another example, the constant region comprises T350V, L351Y, F405A and Y407V mutations (hole). Exemplary constant region amino acid substitutions are numbered according to the EU numbering system.

### Other Antibodies and Antibody Fragments

The present disclosure also contemplates other antibodies and antibody fragments, such as:
(i) minibodies, e.g., as described in U.S. Pat. No. 5,837,821;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980;
(iv) “key and hole” bispecific proteins as described in U.S. Pat. No. 5,731,168; and
(v) Fab₃ (e.g., as described in EP19930302894).

### Stabilised Proteins

Proteins of the present disclosure can comprise an IgG₄ constant region or a stabilized IgG₄ constant region. The term “stabilised IgG₄ constant region” will be understood to mean an IgG₄ constant region that has been modified to reduce Fab arm exchange or the propensity to undergo Fab arm exchange or formation of a half-antibody or a propensity to form a half antibody. “Fab arm exchange” refers to a type of protein modification for human IgG₄, in which an IgG₄ heavy chain and attached light chain (half-molecule) is swapped for a heavy-light chain pair from another IgG₄ molecule. Thus, IgG₄ molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG₄ antibody dissociates to form two molecules each containing a single heavy chain and a single light chain.

In one example, a stabilised IgG₄ constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG₄, this residue is generally a serine. Following substitution of the serine for proline, the IgG₄ hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG₁ according to the numbering system of Kabat. Hinge regions of other IgG isotypes may be aligned with the IgG₁ sequence by placing the first and last cysteine residues forming inter-heavy chain disulphide (S—S) bonds in the same positions (see for example WO2010080538).

### Constant Domain Fusions

The present disclosure encompasses a protein comprising a variable region of an antibody and a constant region or Fc or a domain thereof, e.g., C_{H}2 and/or C_{H}3 domain. Suitable constant regions and/or domains will be apparent to the skilled artisan and/or the sequences of such polypeptides are readily available from publicly available databases. Kabat et al also provide description of some suitable constant regions/domains.

Constant regions and/or domains thereof are useful for providing biological activities such as, dimerization, extended serum half-life e.g., by binding to FcRn (neonatal Fc Receptor), antigen dependent cell cytotoxicity (ADCC), complement dependent cytotoxicity (CDC, antigen dependent cell phagocytosis (ADCP).

The present disclosure also contemplates proteins comprising mutant constant regions or domains, e.g., as described in U.S. Pat. Nos. 7,217,797; 7,217,798; or US20090041770 (having increased half-life) or US2005037000 (increased ADCC).

### Preparation of the Pharmaceutical Formulation

As described herein, the formulations of the present disclosure comprise an organic acid buffer, a non-ionic surfactant and at least one amino acid stabiliser. In some examples, the formulation has a pH of 5.0 to 6.0. Preparation of the pharmaceutical formulation is performed according to standard methods known in the art and/or according to methods described herein.

### Organic Acid Buffers

The skilled person will understand that organic acid buffers suitable for use in the present disclosure comprise one or more carboxylic acid or acid phenolic groups without basic amino groups. In addition to the buffering capacity provided by the acidic groups, such organic buffers used herein can contain additional ionisable functionality provided by, for example, an amino group.

It will be apparent to the skilled person that buffers suitable for use in the present disclosure will be stable and effective at the desired pH and will provide sufficient buffer capacity to maintain the desired pH over the range of conditions to which it will be exposed during formulation and storage of the product. For example, a stable buffer will provide thermal aggregation stability (e.g., during freeze/thaw or elevated temperatures), not be affected by oxidation of physical degradation (e.g., insoluble particulate formation) and provide the desired polydispersity (i.e., particle distribution). Suitable buffers will not form deleterious complexes with metal ions, be toxic, or unduly penetrate, solubilise, or absorb on membranes or other surfaces. Furthermore, the skilled person will recognise that such buffers should not interact with other components of the composition in any manner which decreases their availability or effectiveness. Additionally, the buffering agent of the pharmaceutical formulation must be safe for administration, compatible with other components of the composition over the shelf-life of the product, and acceptable for administration to the subject.

Suitable organic acid buffers for use in the present disclosure will be apparent to the skilled person and include, for example, histidine buffers (e.g., histidine chloride, histidine acetate, histidine phosphate, histidine sulfate, etc.), glutamate buffers (e.g., monosodium glutamate, etc.), citrate buffers (e.g. monosodium citrate-disodium citrate mixture, citric acid-trisodium citrate mixture, citric acid-monosodium citrate mixture, etc.), succinate buffers (e.g. succinic acid-monosodium succinate mixture, succinic acid-sodium hydroxide mixture, succinic acid-disodium succinate mixture, etc.), tartrate buffers (e.g. tartaric acid-sodium tartrate mixture, tartaric acid-potassium tartrate mixture, tartaric acid-sodium hydroxide mixture, etc.), fumarate buffers (e.g. fumaric acid-monosodium fumarate mixture, fumaric acid-disodium fumarate mixture, monosodium fumarate-disodium fumarate mixture, etc.) gluconate buffers (e.g. gluconic acid-sodium gluconate mixture, gluconic acid-sodium hydroxide mixture, gluconic acid-potassium gluconate mixture, etc.), oxalate buffers (e.g. oxalic acid-sodium oxalate mixture, oxalic acid-sodium hydroxide mixture, oxalic acid-potassium oxalate mixture, etc.), lactate buffers (e.g. lactic acid-sodium lactate mixture, lactic acid-sodium hydroxide mixture, lactic acid-potassium lactate mixture, etc.) and acetate buffers (e.g. acetic acid-sodium acetate mixture, acetic acid-sodium hydroxide mixture, etc.).

In one example of the present disclosure, the organic acid buffer is selected from the group consisting of a histidine buffer, a glutamate buffer, a succinate buffer and a citrate buffer. For example, the organic acid buffer is a histidine buffer. For example, the organic acid buffer is L-histidine.

Methods of assessing the suitability of buffers will be apparent to the skilled person and/or described herein and include, for example, differential scanning fluorimetry and dynamic light scattering.

### Non-Ionic Surfactants

The amount of non-ionic surfactant added to the pharmaceutical formulation will be apparent to the skilled person and is in an amount such that it suppresses aggregation (e.g., by preventing surface denaturation), increases stabilisation (e.g., during thermal and/or physical stress), minimises the formation of particulates in the formulation (e.g., sub-visible and/or visible particle formation), reduces surface adsorption and/or assists in protein refolding.

Suitable non-ionic surfactants for use in the present disclosure will be apparent to the skilled person and include, for example, polyoxyethylensorbitan fatty acid esters (e.g., polysorbate 20 and polysorbate 80), polyethylene-polypropylene copolymers, polyethylene-polypropylene glycols, polyoxyethylene-stearates, polyoxyethylene alkyl ethers, e.g. polyoxyethylene monolauryl ether, alkylphenylpolyoxyethylene ethers (Triton-X), polyoxyethylene-polyoxypropylene copolymer (Poloxamer, Pluronic), sodium dodecyl sulphate (SDS).

In one example of the present disclosure, the non-ionic surfactant is selected from the group consisting of polyoxyethylensorbitan fatty acid esters and polyoxyethylene-polyoxypropylene copolymers. For example, the polyoxyethylensorbitan fatty acid ester is polyoxyethylene sorbitan monooleate (i.e., polysorbate 80) or polyoxyethylene sorbitan monolaurate (polysorbate 20).

### Amino Acid Stabilisers

The amount of amino acid stabiliser(s) added to the pharmaceutical formulation will be apparent to the skilled person and is in an amount that such that it reduces thermal and/or physical stress (e.g., freeze/thaw or agitation), and/or confers or enhances stability of the protein.

Suitable amino acids for use in the present disclosure will be apparent to the skilled person and include, for example, glycine, alanine, valine, leucine, isoleucine, methionine, threonine, phenylalanine, tyrosine, serine, cysteine, histidine, tryptophan, proline, aspartic acid, glutamic acid, arginine, lysine, ornithine and asparagine and salts thereof.

In one example of the present disclosure, the at least one amino acid is selected from the group consisting of proline and arginine. For example, the at least one amino acid stabiliser includes proline or a salt form thereof. For example, the at least one amino acid stabiliser includes arginine or a salt form thereof. For example, the amino acid stabilisers are proline and arginine or a salt form thereof.

### Protein Production

Methods of producing and obtaining proteins for use in the formulation described herein will be known to those skilled in the art. For example, in the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant proteins are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's F10 (Sigma), Minimal Essential Medium ((MEM), (Sigma), RPMl-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Where a protein (e.g., antibody) is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

### Assaying the Pharmaceutical Formulation and Proteins of the Disclosure

High concentration pharmaceutical formulations of the present disclosure are readily screened for physical and biological activity and/or stability using methods known in the art and/or as described below.

### Neutralization Assays

For proteins or compounds that bind to VEGF-B and inhibit signaling, a neutralization assay can be used.

In one example, a neutralization assay involves contacting VEGF-B with a compound in the presence or absence of detectably labeled soluble VEGF-R1 or contacting detectably labeled VEGF-B with a compound in the presence or absence of a cell expressing VEGF-R1 or a soluble VEGF-R1. The level of VEGF-B bound to the VEGF-R1 is then assessed. A reduced level of bound VEGF-B in the presence of the compound compared to in the absence of the compound indicates the compound inhibits VEGF-B binding to VEGF-R1 and, as a consequence VEGF-B signaling.

Another neutralization assay is described in WO2006/012688 and involves contacting a fragment of VEGF-R1 comprising the second Ig-like domain immobilized on a solid support with a subsaturating concentration of recombinant VEGF-B pre-incubated with a compound. Following washing to remove unbound protein, the immobilized protein is contacted with anti-VEGF-B antibody and the amount of bound antibody (indicative of immobilized VEGF-B) determined. A compound that reduces the level of bound antibody compared to the level in the absence of the compound is considered an inhibitor of VEGF-B signaling.

In another example, a compound that inhibits VEGF-B signaling is identified using a cell dependent on VEGF-B signaling for proliferation, e.g., a BaF3 cell modified as described in WO2006/012688 to express a chimeric receptor incorporating the intracellular domain of the human erythropoietin receptor and the extracellular domain of VEGF-R1. Cells are cultured in the presence of VEGF-B and in the presence or absence of a compound. Cell proliferation is then assessed using standard methods, e.g., colony formation assays, thymidine incorporation or uptake of another suitable marker of cell proliferation (e.g., a MTS dye reduction assay). A compound that reduces the level of proliferation in the presence of VEGF-B is considered an inhibitor of VEGF-B signaling.

Compounds can also be assessed for their ability to bind to VEGF-B using standard methods. Methods for assessing binding to a protein are known in the art, e.g., as described in Scopes (In: Protein purification: principles and practice, Third Edition, Springer Verlag, 1994). Such a method generally involves labeling the compound and contacting it with immobilized VEGF-B. Following washing to remove non-specific bound compound, the amount of label and, as a consequence, bound compound is detected. Of course, the compound can be immobilized and the VEGF-B labeled. Panning-type assays can also be used. Alternatively, or additionally, surface plasmon resonance assays can be used.

### Expression Assays

A compound that reduces or prevents expression of VEGF-B is identified by contacting a cell with the compound and determining the level of expression of the VEGF-B. Suitable methods for determining gene expression at the nucleic acid level are known in the art and include, for example, quantitative polymerase chain reaction (qPCR) or microarray assays. Suitable methods for determining expression at the protein level are also known in the art and include, for example, enzyme-linked immunosorbent assay (ELISA), fluorescence linked immunosorbent assay (FLISA), immunofluorescence or Western blotting.

### In Vivo Assays

Compounds described herein can be tested for activity in animal models. In one example, the animal model is a model of brain inflammation. For example, the model is a model of experimental autoimmune encephalomyelitis (EAE). This is a well-established model in which a mouse or rat is immunized with a myelin sheath protein or peptide derived therefrom (e.g., MOG, MBP or PLP) and an immune response is generated against the protein thereby inducing a model of multiple sclerosis. Exemplary EAE models are reviewed in, for example Tsunoda and Fujinami, J. Neuropathol. Exp. Neurol. 55: 673-686, 1996.

### Visual Appearance

Pharmaceutical formulations encompassed by the present disclosure can be assessed for visual appearance to determine, for example, the colour and clarity or for the presence of visible particles.

### Dynamic Light Scattering

In one example, the particle size distribution is assessed using dynamic light scattering (DLS). DLS measures light scattered from particles based on Brownian motion and relies on differences in the index of refraction between the particle and the formulation. For example, the fluctuation of light intensity using a digital correlator is measured. The correlation functions are fitted into an analytical program (e.g., Malvern Zetasizer software) to calculate the particle size distribution. For the determination of Z-average hydrodynamic diameter, a cumulants analysis and the Stokes Einstein equation is performed using e.g., the viscosity of water (0.8872 mPa*s) at 25° C. The polydispersity index can also be obtained from the same cumulants analysis. Modality of fit is evaluated based on plots of size distribution versus intensity: modality can be described as monomodal (i.e., one peak) or multimodal (i.e., two or more peaks).

### Micro-Flow Imaging

In one example, sub-visible particles are assessed using micro-flow imaging (MFI). For example, digital images of particles suspended in a fluid are captured and automatically analysed for particle parameters, such as aspect ratio (AR) and intensity. The size (e.g., in μm) and count (i.e., number of particles per ml) can also be obtained. According to this method the data are morphologically categorised as proteinaceous (i.e., circular) and non-proteinaceous (i.e., non-proteinaceous particles such as air bubbles or silicone oil droplets) and a ratio of the non-proteinaceous particles to proteinaceous particles (i.e., the circular fraction) can be determined. A low circular fraction value indicates that the test article is comprised of mostly non-circular, likely proteinaceous particles.

### Size Exclusion Chromatography

In one example, aggregates/HMWS are assessed using size exclusion chromatography (SEC or SE-HPLC) which separates lower and higher molecular mass variants of the protein, as well as any impurities. According to this method, the results are described as the summation of aggregation peaks (APs) and summation of degradation peaks (DPs). For example, the identity of a pharmaceutical formulation of the present disclosure can be determined by comparing the chromatographic retention time of the major peaks with the retention time of the major peak of a reference standard.

### Differential Scanning Fluorimetry (DSF)

In one example, thermal stability of the pharmaceutical formulation of the present disclosure is assessed using differential scanning fluorimetry (DSF). DSF is a fluorescence-based assay using real-time PCR to monitor thermally induced protein denaturation by measuring changes fluorescence of a dye that binds preferentially to unfolded protein. For example, thermal unfolding and aggregation are monitored by changes in intrinsic protein fluorescence and static light scattering, respectively, as a function of temperature. According to this method, the midpoint of thermal transition (Tₘ) and onset of melting temperature (Tₒₙₛₑₜ) are determined by monitoring intrinsic fluorescence. The onset of aggregation temperature (T_{agg}) are determined by monitoring static light scattering, e.g., at 266 nm and 473 nm. Samples of the pharmaceutical formulation can be assessed across a range of temperatures, (e.g., 20° C.-95° C.) with a temperature increase at the rate of e.g., 0.5° C./min.

### Capillary Gel Electrophoresis

In one example, the pharmaceutical formulation of the present disclosure is assessed for stability and/or total accumulation of impurities using capillary gel electrophoresis (CGE). For example, both reduced-CGE (R-CGE) and non-reduced-CGE (NR-CGE) may be performed. In one example, R-GCE and NR-CGE are carried out using a capillary electrophoresis system (e.g., Beckman P/ACE MDQ or PA800) with a capillary length of e.g., 20.2 cm and 10 cm respectively from inlet to detection window, temperature control from e.g., 20 to 40° C. (±2° C.) and detector at e.g., 488 nm excitation.

### Gibbs Free Energy (ΔG_{trend}; HUNK)

In one example, the chemical stability and aggregation behaviour of a pharmaceutical formulation of the present disclosure is evaluated by the change in the Gibbs free energy or ΔG_{trend} (HUNK) analysis. The ΔG_{trend} analysis measures the relationship between ΔG of protein unfolding and protein aggregation as a function of protein concentration. In the absence of aggregation, the ΔG of protein unfolding is a unimolecular process independent of protein concentration. If a change in ΔG is observed as a function of protein concentration, it signifies presence of aggregation. According to this method, there are two possible relationships between ΔG of protein unfolding and protein concentration if aggregation occurs:
1. ΔG_{trend} increases with protein concentration: This relationship indicates the presence of native state aggregation—the ΔG of protein unfolding increases (becomes more positive) as a function of protein concentration (i.e., concentration of native protein aggregates increases as a function of protein concentration); or
2. ΔG_{trend} decreases with protein concentration: This relationship indicates the presence of denatured state aggregation—the ΔG of protein unfolding decreases (become less positive) as a function of protein concentration (i.e., concentration of denatured protein aggregates increases as a function of protein concentration).

In a HUNK experiment the ΔG of protein unfolding is determined isothermally by measuring changes in a protein's intrinsic fluorescence spectrum (i.e., emission from tryptophan residues) as it unfolds in the presence of increasing amounts of denaturant.

In one example, ΔG_{trend} is determined by measuring ΔG of the protein unfolding at varying concentrations (e.g., 0.25, 0.6, 2.5, 6.0, 25.0 mg/ml) diluted to target concentration in a buffer of the pharmaceutical formulation of the disclosure. Each concentration level is titrated with increasing denaturant concentration (e.g., 32-point curve spanning urea concentration 2.00-8.74 M) while fluorescence spectra is measured from 300-500 nm (excitation 280 nm) with a slit width of 10 nm. The emission spectrum wavelength ratio of 350 nm/330 nm is plotted against urea concentration for each sample concentration level, and ΔG of protein unfolding determined using a 2 state (i.e., one transition) model fit. Determined ΔG values are plotted against sample concentration to determine ΔG_{trend}.

### Capillary Electrophoresis

In some examples, the formulation is assessed by capillary electrophoresis (CE). For example, the formulation may be assessed by capillary electrophoresis with sodium dodecylsulfate (CE-SDS) under non-reducing conditions to determine the proportion of LMWS present. Capillary electrophoresis is a separation method performed in submillimeter diameter capillaries and in micro- and nanofluidic channels. Proteins migrate through electrolyte solutions under the influence of an electric field. In the presence of SDS, proteins are denatured and are separated on the basis of their molecular weight. This enables the detection of LMWS present in the formulation, for example LMWS produced upon degradation (e.g., proteolytic degradation) of the protein.

### Turbidity Assessed by Absorbance at 550 nm

In one example, the turbidity of the pharmaceutical formulation of the present disclosure is assessed. For example, the turbidity is assessed using a spectrophotometer and measuring the absorbance at 550 nm.

### Syringeability

In one example, the syringeability of the pharmaceutical formulation of the present disclosure is assessed. For example, the formulation is expelled with a 2 ml syringe, 10 ml syringe, or left untreated as a pre-expulsion control. According to this method, the syringe plunger is pushed through the 2 ml syringes at a linear speed of 0.2 in/min and through the 10 ml syringes at 0.6 in/min until the plunger reaches the bottom and reaches the force of 30 N. Break-loose (BF) and glide (GF) forces are measured during expulsion and used to assess application suitability. Break-loose force describes the force required to initiate movement of the plunger (the initial 0.3 mm for 2 ml syringe and 0.5 mm for 10 ml syringe). Glide force Max refers to the maximum friction force required to sustain plunger movement. The maximum force value is measured from the end of the break loose region to the end of the glide force region (26 mm for 2 ml syringe and 24 mm for 10 ml syringe) prior to the point where the force reaches 30 N).

### Uses of the Pharmaceutical Formulation

As discussed herein, the present disclosure provides a method of treating or preventing or delaying progression of a disease or condition in a subject, comprising administering a pharmaceutical formulation of the present disclosure to the subject. In one example, the present disclosure provides a method of treating or preventing or delaying progression of a disease or condition in a subject in need thereof.

The present disclosure also provides for use of a pharmaceutical formulation of the present disclosure for treating or preventing or delaying progression of a disease or condition in a subject comprising administering the pharmaceutical formulation of the present disclosure to the subject. In one example, the present disclosure provides for use of a pharmaceutical formulation of the present disclosure for treating or preventing or delaying progression of a disease or condition in a subject in need thereof.

In one example, the disease or condition is selected from the group consisting of nephropathy, a wound, a non-alcoholic fatty liver disease (NAFLD) or complication thereof, a stroke, a wasting disorder, obesity, insulin resistance, diabetes, a cardiovascular disorder, metabolic syndrome, and combinations thereof.

In one example, the disease or condition is nephropathy. For example, the nephropathy is diabetic nephropathy (i.e., nephropathy associated diabetes). For example, the subject suffers from nephropathy associated with type 1 diabetes. For example, the subject suffers from nephropathy associated with type 2 diabetes. In another example, the subject suffers from diabetes and microalbuminuria or diabetes and macroalbuminuria.

In one example, the disease or condition is a wound. The wound can be a chronic, acute or normal wound. In one example, the wound is chronic. In one example, a subject suffers from a wound that is infected and/or ischemic. In another example, the wound is a full-thickness wound. In a further example, the wound is a diabetic ulcer, e.g., a diabetic foot ulcer.

In one example, the disease or condition is a non-alcoholic fatty liver disease (NAFLD) or a complication thereof. In one example, the NAFLD is selected from the group consisting of hepatic steatosis (e.g., isolated hepatic steatosis), non-alcoholic steatohepatitis (NASH), cirrhosis, NASH-derived cirrhosis, NASH-associated cirrhosis, NASH-associated hepatic fibrosis. In one example, the NAFLD is hepatic steatosis or NASH or cirrhosis or hepatic fibrosis. In one example, the NAFLD is severe hepatic steatosis. In one example, the complication of the NAFLD is hepatocellular carcinoma.

In one example, the disease or condition is a stroke. For example, the stroke is an ischemic stroke. For example, the stroke is a cerebral ischemic stroke.

In one example, the disease or condition is a wasting disorder. In one example, the wasting disorder is selected from the group consisting of cachexia, unintended body weight loss, fat wasting and anorexia. For example, the cachexia is selected from the group consisting of cancer cachexia, chronic kidney disease cachexia and diabetic cachexia.

In one example, the disease or condition is obesity.

In one example, the disease or condition is insulin resistance.

In one example, the disease or condition is diabetes. For example, type I or type II diabetes.

In one example, the disease or condition is a cardiovascular disorder. In one example, the cardiovascular disease is elevated blood pressure, atherosclerosis, heart failure or a cardiovascular event such as acute coronary syndrome, myocardial infarction, myocardial ischemia, chronic stable angina pectoris, unstable angina pectoris, angioplasty, stroke, transient ischemic attack, claudication(s), or vascular occlusion(s).

In one example, the disease or condition is a metabolic syndrome.

In some examples, the subject is administered an effective amount of the protein in the formulation of the present disclosure. An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change in a factor associated with a disease or condition as hereinbefore described. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number. The effective amount can be administered in a single dose or in a dose repeated once or several times over a treatment period.

In some examples, the subject is administered a therapeutically effective amount of the protein in the formulation of the present disclosure. A “therapeutically effective amount” is at least the minimum concentration required to effect a measurable improvement of a particular disease or condition. A therapeutically effective amount herein may vary according to factors such as the disease state, age, sex, and weight of the patient, and the ability of the antibody or antigen binding fragment thereof to elicit a desired response in the individual. A therapeutically effective amount is also one in which any toxic or detrimental effects of the protein are outweighed by the therapeutically beneficial effects.

In one example, the pharmaceutical formulation of the present disclosure is administered to the subject in an amount to reduce the severity of the disease or condition in the subject.

In one example, the subject is at risk of developing a disease or condition described herein. A subject is at risk if he or she has a higher risk of developing a disease or condition described herein than a control population. The control population may include one or more subjects selected at random from the general population (e.g., matched by age, gender, race and/or ethnicity) who have not suffered from or have a family history of a disease or condition. A subject can be considered at risk for a disease or condition if a “risk factor” associated with a disease or condition is found to be associated with that subject. A risk factor can include any activity, trait, event or property associated with a given disorder, for example, through statistical or epidemiological studies on a population of subjects. A subject can thus be classified as being at risk for a disease or condition even if studies identifying the underlying risk factors did not include the subject specifically.

In one example, the subject is at risk of developing a disease or condition described herein and the pharmaceutical formulation of the present disclosure is administered before or after the onset of symptoms of the disease or condition. In one example, the pharmaceutical formulation is administered before the onset of symptoms of the disease or condition. In one example, the pharmaceutical formulation is administered after the onset of symptoms of the disease or condition. In one example, the pharmaceutical formulation of the present disclosure is administered at a dose that alleviates or reduces one or more of the symptoms of the disease or condition in a subject at risk.

The methods of the present disclosure can be readily applied to any form of a disease or condition described herein in a subject. In one example, a method of the disclosure reduces any symptom of the disease or condition known in the art and/or described herein. As will be apparent to the skilled person a “reduction” in a symptom of a disorder in a subject will be comparative to another subject who also suffers from a disorder but who has not received treatment with a method described herein. This does not necessarily require a side-by-side comparison of two subjects. Rather population data can be relied upon. For example, a population of subjects suffering from the disease or condition who have not received treatment with a method described herein (optionally, a population of similar subjects to the treated subject, e.g., age, weight, race) are assessed and the mean values are compared to results of a subject or population of subjects treated with a method described herein.

A method of the present disclosure may also include co-administration of the pharmaceutical formulation according to the disclosure together with the administration of another therapeutically effective agent for the prevention or treatment of a disease or condition described herein.

In one example, the pharmaceutical formulation of the disclosure is used in combination with at least one additional known compound or therapy which is currently being used or is in development for treating or preventing or delaying progression of a disease or condition (e.g., as described herein).

As will be apparent from the foregoing, the present disclosure provides methods of concomitant therapeutic treatment of a subject, comprising administering to a subject in need thereof an effective amount of a first agent and a second agent or therapy, wherein the first agent is a pharmaceutical formulation of the present disclosure, and the second agent or therapy is also for the prevention or treatment of a disease or condition as described herein.

As used herein, the term “concomitant” as in the phrase “concomitant therapeutic treatment” includes administering a first agent in the presence of a second agent or therapy. A concomitant therapeutic treatment method includes methods in which the first, second, third or additional agents/therapies are co-administered. A concomitant therapeutic treatment method also includes methods in which the first or additional agents are administered in the presence of a second or additional agent or therapy, wherein the second or additional agent or therapy, for example, may have been previously administered. A concomitant therapeutic treatment may be executed step-wise by different actors. For example, one actor may administer to a subject a first agent and as a second actor may administer to the subject a second agent or therapy and the administering steps may be executed at the same time, or nearly the same time, or at distant times, so long as the first agent (and/or additional agents) are after administration in the presence of the second agent or therapy (and/or additional agents or therapies). The actor and the subject may be the same entity (e.g. a human).

### Kits and Other Compositions of Matter

Another example of the disclosure provides kits containing a pharmaceutical formulation of the present disclosure useful for the treatment or prevention or delaying progression of a disease or condition as described above.

In one example, the kit comprises (a) a container comprising a pharmaceutical formulation of the present disclosure; and (b) a package insert with instructions for treating or preventing or delaying progression of a disease or condition in a subject.

In one example, the kit comprises (a) at least one pharmaceutical formulation of the present disclosure; (b) instructions for using the kit in treating or preventing or delaying progression of the disease or condition in the subject; and (c) optionally, at least one further therapeutically active compound or drug.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating a disease or condition as described herein and may have a sterile access port (for example, the container may be an intravenous solution bag or a vial having a stopper pierceable by a hypodermic injection needle). The label or package insert indicates that the composition is used for treating a subject eligible for treatment, e.g., one having or predisposed to developing a disease or condition as described herein, with specific guidance regarding dosing amounts and intervals of the pharmaceutical formulation and any other medicament being provided. The kit may further include other materials desirable from a commercial and user standpoint, including filters, needles, and syringes. In some examples of the present disclosure, the formulation can be present in an injectable device (e.g., an injectable syringe, e.g., a prefilled injectable syringe). The syringe may be adapted for individual administration, e.g., as a single vial system including an autoinjector (e.g., a pen-injector device). In one example, the injectable device is a prefilled pen or other suitable autoinjectable device, optionally with instruction for use and administration.

The kit optionally further comprises a container comprising a second medicament, wherein the pharmaceutical formulation is a first medicament, and which article further comprises instructions on the package insert for treating the subject with the second medicament, in an effective amount. The second medicament may be a therapeutic protein set forth above.

In one example, the disclosure provides a prefilled syringe or autoinjector comprising a formulation of the present disclosure. In one example, the prefilled syringe is a glass luer syringe with plunger.

In one example, the disclosure provides a vial comprising a formulation of the disclosure.

The present disclosure includes the following non-limiting Examples.

### EXAMPLES

### Example 1: Materials and Methods

The materials used for the following Examples, their catalogue numbers and suppliers are listed in Table 1.

TABLE 1
Materials used for the examples

| Material | | Catalogue Number | Supplier |
|---|---|---|---|
| 2 mL Glass Vials, 13 mm neck | | 1551745 | Schott Igar glass |
| 13 mm Stopper | | 19700004 | West |
| 13 mm cap | | 54133014 | West |
| Nalgene Biocontainers | | | Thermo Scientific ™ |
| 5 | mL | 2035-0005 | |
| 10 | mL | 2035-0010 | |
| 20 | mL | 2035-0020 | |
| 30 | mL | 342020-0030 | |
| 250 | mL | 342020-0250 | |
| Nunc ™ 96 Well PP DeepWell ™ | | 278743 | Thermo Scientific ™ |
| Storage Plates | | | |
| Pre-Slit Well Cap for 96 Well PP | | 276011 | Thermo Scientific ™ |
| Plate Non Sterile, Silicone | | | |
| Matrix ™ 2D Barcoded Open-Top | | | Thermo Scientific ™ Matrix |
| V bottom, PP Storage Tubes | | | |
| 0.5 | mL | 3735 | |
| 1.4 | mL | 3792 | |
| Sepra Seals | | MAT4464 | Thermo Scientific ™ |
| ambr ® CF filter (30,000 MWCO), | | 3MA1445910 | Sartorius Stedim Lab Ltd |
| Hydrosart ® | | | |
| BD Syringe | | | Becton Dickinson |
| 10 | mL | 302143 | |
| 50 | mL | 300865 | |
| Cellstar ® Tubes | | | Greiner Bio-One |
| 15 | mL | 188271 | |
| 50 | mL | 227261 | |

| Tecan Pure disposable trough, PP gray, | 30055743 | Tecan ® |
|---|---|---|
| 25 mL disposable trough | | |
| Pipette - 25 mL | 47525 | Sterilin ™ |
| Pipette - 50 mL | 768 180 | Greiner Bio-One |
| Pellicon ® 3 with Ultracel ® 30 kDa | P3C030D00 | Merck |
| membrane, D screen, 88 cm | | |
| Millex-GP Syringe Filter Unit, 0.22 μm | SLGP033RS | Merck |
| Millipore Steritop, 0.22 μm | S2GPT10RE | Merck |
| Hydrochloric acid, 6M | 1.10164 | Merck |
| Sodium Hydroxide - 5M | 1.37041 | Merck |
| Sodium Hydroxide - 4M | 1048 | ACR Chemical Reagents |
| L-Histidine | 1.04352 | Merck |
| Sodium chloride | 106404 | Merck |
| L-Glutamic acid | 1.01791 | Merck |
| L-Proline | 1.07430 | Merck |
| L-Arginine | A5006 | Sigma-Aldrich ® |
| | A4474 | |
| L-Arginine•HCl | 1.01544 | Merck |
| L-Methionine | M8439 | Sigma-Aldrich ® |
| Meglumine | 1.06143 | Merck |
| Acetic acid (glacial) 100% | 1.00056 | Merck |
| (S)-Lactic acid about 90% | 1.00366 | Merck |
| Calcium Chloride Dihydrate | 1.37101 | Merck |
| L-Isoleucine | I5281 | Sigma-Aldrich ® |
| Polysorbate 80 | HX2 | NOF Corporation |
| Tungsten | N/A* | Beckton Dickson |
| Dow Corning ® 360 Medical fluid, | DC360 | Dow Corning |
| 1000 cSt | | |

*Prepared and supplied by Beckton Dickson on request

### Preparation of Formulations

The bulk anti-VEGFB antibody (CSL346) material was buffer exchanged into the formulations and pHs using TFF, with at least 6 buffer exchanges cycles. All formulations were concentrated up, and when final concentration was at or in excess of the target concentration (≥200 mg/mL CSL346) material was recovered. The protein concentration was measured, surfactant added to the target concentration, and all formulations diluted to the target protein concentration with the formulation diluent. If maximum concentration was below the target no further dilution was performed. Formulations were 0.2 μm filtered and stored in the appropriate storage container e.g. Matrix containers, biocontainers (Nalgenes) or glass vials at various fill volumes.

### Visual Appearance

Visual appearance was conducted in an inspection station equipped with a white and black background and fluorescent light. Formulations in vials were gently swirled without producing bubbles then inspected for colour, clarity and the presence of visible particles. Inspections were conducted by a single inspector, with visible particles or changes from initial, where present, confirmed by a second independent inspector.

### pH Measurements

The pH of the formulations was measured using a Mettler Toledo SevenExcellence pH meter equipped with an InLab® Ultra Micro ISM electrode.

### UV Spectroscopy

Protein concentration was measured by using A280/UV determination on the formulations, with the mean value of replicate measurements calculated via two methods:
neat on the Lunatic Spectrophotometer, with measurements conducted in duplicate or triplicate
gravimetric dilution on the Shimadzu UV-1700 Spectrophotometer or Agilent Cary 3500 Spectrophotometer, with measurements conducted in duplicate.

### Size Exclusion (SE)-High Performance Liquid Chromatography (HPLC)

SE-HPLC was used to determine the protein aggregation profile of then formulations. Intact protein was detected at 280 nm with monomer species, high molecular weight species (HMWS, aggregates) and low molecular weight species (LMWS, fragments) reported as a relative area %. Internal and external references were used to validate the run. This was performed with a Dionex system (Ultimate 3000) with an Acquity BEH200 column (Waters, 1.7 μm, 4.6×150 mm) to analyse the samples. Samples were diluted to 10 g/L in appropriate buffer and 2 μL was injected. Separation was performed under isocratic conditions at a flow rate of 0.2 mL/min. Mobile phase consisted MOPS/NaCl buffer (pH 7.0) with a run time of 15 min.

### Osmolality Measurements

Osmolality of the formulations was measured by using a Vapro 5600 vapour pressure osmometer. Sample volumes were 10 μL. Measurements were conducted in duplicate or triplicate and the mean values of the measurements calculated.

### pH Measurement

pH was measured using a Mettler Toledo SevenExcellence pH meter equipped with an InLab® Ultra Micro ISM electrode.

### Analysis of Polysorbate 80 (PS80)

Reverse Phase-HPLC (RP-HPLC) was used to quantify the amount of PS80 at the initial time point (TO) in the different formulations. PS80 standard and the samples were treated with ethanol followed by 0.1M KOH 5 at 40° C. followed by sample analysis of oleic acid resulting from hydrolysis by a RP-HPLC method. A Dionex (Ultimate 3000) System (or equivalent) equipped with a Nova-Pak® C18 3.9×150 mm, 4 μm reverse phase column (Waters) was used to analyse the samples. Injection volume was 15 μL and separation was conducted using an isocratic method at 2.0 ml/min. Mobile phase was 80% acetonitrile with 20% potassium dihydrogen phosphate buffer at pH 2.8. Column temperature was set to 40° C. Species were detected at 190 nm, and quantified using a standard calibration curve generated by the PS80 standard solutions. Data is reported as 35% (w/v) of PS80.

### Amino Acid Excipients (AAE)

Amino acid excipient levels are measured using RP-HPLC. Samples are derivatised using the Waters AccQ.Tag™ Ultra Derivatisation kit. The derivatised amino acids are then separated by reverse phase on a Dionex (Ultimate 3000) System (or equivalent) equipped with a Waters AccQ. Tag Ultra 2.1×100 mm column and detected by ultraviolet spectrophotometer at 260 nm. Injection volume was 1 μL and separation was conducted using a gradient method at 0.7 ml/min. Mobile phase A was AccQ. Tag Ultra Eluent A diluted 1 in 20 and Mobile Phase B was AccQ. Tag Ultra Eluent B. Column temperature was set to 55° C. and elution time was 10.5 minutes. The concentration of amino acids in each sample is calculated using their peak area and the calibration curve.

### Melting Temperature (Tm) and Aggregation Onset Temperature (Tagg)

UNcle was used to determine Tm and Tagg at the initial time point. A linear thermal ramp at a rate of 0.5° C. per minute was applied to the samples from 20 to 90° C. The Tm of the protein in each formulation was measured using intrinsic fluorescence with an excitation wavelength of 266 nm. Analysis was performed using the Barycentric Mean (BCM) method to determine the Tm. The Tagg was simultaneously measured using static light scattering (SLS) at 473 nm. The Tagg was determined based on the change in the SLS counts during the thermal ramp.

### Relative Viscosity by Dynamic Light Scattering (DLS)

The viscosity of the bulks and formulations was measured using the UNcle DLS method. A polystyrene bead standard (200 nm diameter) was sonicated for 1 min before use, and added at a 1 in 100 ratio with the protein sample then vortexed for 2-3 seconds. Samples were always vortexed directly before pipetting into the UNI cassettes for measurement. A Milli-Q water sample (containing beads at a 1 in 100 ratio) was included in each UNI cassette as a control, and 8.7 μL of each sample run in triplicates within 30 minutes of preparation using the DLS viscosity application. A 60 nm radius and 5% intensity cut-off filter was used to filter out peaks measured due to the beads or contaminants.

### Viscosity (Rheometer)

The viscosity of the formulations was measured using the Anton Paar MCR102 Rheometer using a cone and plate system. Measurements were performed at 20° C. using the CP 40-0.3 measuring system, with samples run in triplicate as 130 μL load volumes. Viscosity measurements were recorded at 1 second intervals over 90 seconds at a constant shear rate of 100 s-1. Data from the final 30 seconds of each 90 second run was used for averaging.

### Density

The density of the protein samples was determined using the MettlerToledo DA-100M Density Meter at 20° C. by measuring the frequency of electromagnetically induced oscillations of the glass U-tube over a defined period of time. Approximately 1 mL of sample was used for density measurements after calibration of the instrument with air and degassed milli-Q water at 20° C.

### Multi Attribute Method (MAM)

MAM is mass spectrometry-based, bottom-up method which concurrently monitors several molecular attributes. MAM was used to estimate the relative abundance of degradation post-translational modifications (PTMs) in formulation samples. Samples were tryptic digested and subsequently subjected to LC-MS analysis. Tryptic peptides were separated using ThermoScientific Ultimate 3000 HPG (or equivalent) LC system equipped with Waters CSH C18 1×50 mm, 1.7 μm, 130 Å (or equivalent) column and hyphenated to ThermoScientific Q Exactive HF mass spectrometer. The mobiles phases A and B used for separation were 0.1% formic acid in distilled water and 0.1% formic acid in Acetonitrile respectively, over a 14 min gradient elution. Approximately 1.2 g of protein was injected at a flow rate 0.2 mL/min and column temperature set to 40° C. MS data acquisition parameters included a full MS-SIM scan type with 240′000 resolutions and 3e6 ΔGC target. Maximum IT was 256 ms, with a scan range of 350 to 2000 m/z, positive polarity, a lock mass of 371.10123 (positive), and a single microscan and scan range. The MS data files were processed, reviewed, and analysed using web-based platform. The relative abundance (%) of degradation PTM is calculated based on area under the curve (AUC) in the extracted ion chromatograms (XIC) of modified and unmodified peptides.

### Capillary Gel Electrophoresis (CGE)

The protein “banding pattern” was obtained by Capillary Gel Electrophoresis, with two potential assays: reduced (CGE-R) and non-reduced (CGE-NR) analyses, based on prior sample treatment. Analysis was performed using a microfluidic LabChip GXII system (Perkin Elmer Australia Pty Ltd) or PA800 (Beckman Coulter). The protein electrophoresis on the microfluidic chip was achieved by integration of the main features of one-dimensional. Denatured proteins were loaded onto the chip directly from a microtiter plate through a capillary sipper. The samples were then electrokinetically loaded and injected into the 14 mm long separation channel containing a low viscosity matrix of entangled polymer solution. The entire sample preparation procedures were performed according to the manufacturers protocol. For non-reduced (NR) samples, protein solution were diluted to 1.5 or 2 g/L with non-reducing buffer and Milli-Q water. Reduced (R) samples were diluted with kit buffer containing dithiothreitol (DTT). Denaturation occurred at 40° C. for 20 min for NR samples and at 80° C. for 15 min for R samples. The PA800 method separates protein species based on their molecular weight, and detection occurs using a UV detector at 214 nm. Under non reducing conditions, prior to analysis the sample is denatured by addition of sodium dodecyl sulphate (SDS) and heat, followed by alkylation of free cysteines using n-ethylmaleimide (NEM). The relative main peak (purity) and low molecular weight species (LMWS; impurity) are measured. Under reducing conditions, prior to analysis the sample is denatured by addition of SDS and heat, followed by reduction of disulphide bonds with β-mercaptoethanol (BME). Results were reported in relative area percentage for LMWS and Intact for NR samples. For R samples, total sum of heavy and light chain fractions were reported.

### Sub-Visible Particle Count Testing

Sub-visible particle counting was performed by Light obscuration using HIAC 9703+ utilising a low volume method of 4×1 mL, with the average of the final 3 runs being calculated and reported as particles ≥10 μm and >25 μm. Analysis of sub-visible particles morphology, size distribution and counts was also performed using a FlowCam Biologics instrument, a Dynamic/flow Imaging Particle Analysis (DIPA) Technique, on selected formulations of interest. A minimum sample volume of 0.25 mL was used. Measurements were conducted in duplicates or triplicate per formulation and the mean values of the measurements calculated and particles counted as 10 to 25 μm and >25 μm.

### Endotoxin

A limulus amebocyte lysate method is used to measure endotoxin by the kinetic chromogenic method. Samples were required to be tested at 4 different dilutions, increasing by 10-fold and results reported from the valid result which has achieved an end-point result and has a PPC recovery of at closest to 100%.

### Potency

The potency ELISA measures in vitro protein binding of CSL346 to its target VEGF-B. A 96-well microtitre plate is coated with VEGF-B (extracellular domain) at a fixed concentration, after which CSL346 antibody at a range of concentrations is added. The plate is washed, and the remaining bound CSL346 antibody is detected by means of horseradish peroxidase (RP) conjugated IgG. Colour development of the HRP substrate is measured in a plate reader at 450 nm, and the data is fitted using a 4-parameter logistic (4PL) regression model. Relative potency is then calculated using parallel line analysis against the reference standard, and the result is reported as percent relative to reference standard.

### Example 2: pH Evaluation

The aim of the experiments described in the following examples was to produce a formulation of CSL346, an antibody that binds to VEGF-B, which had long term stability and was suitable for subcutaneous administration. The starting formulation contained 100 mg/mL CSL346, 20 mM histidine, 100 mM Arginine, 50 mM NaCl, 0.02% w/w PS80 at pH 6.1.

The stability and osmolality of formulations at 150 and 180 mg/mL CSL346, comprising different stabiliser and buffer components across a pH range of 4.5 to 6.5 was evaluated, as shown in Table 2. Stability was assessed after 3 weeks of storage at 35° C. and 6 weeks of storage at 25° C. by SE-HPLC, CGE (R and NR), UV-Vis Spectroscopy and pH. Osmolality, SLS, intrinsic fluorescence and viscosity by DLS was measured at the initial time point only. All formulations comprised 20 mM histidine or glutamic acid, 100 mM arginine and 0.02% w/w PS80 (Table 2).

TABLE 2
HMWS (SE-HPLC) and osmolality of CSL346 formulations comprising different buffer and stabiliser
components across a pH range of 4.5 to 6.5. All formulations contained 100 mM Arginine.

| | | | 150 mg/mL CSL346 | | | 180 mg/mL CSL346 | | | |
|---|---|---|---|---|---|---|---|---|---|
| | | | % HMWS (SE-HPLC) | | | % HMWS (SE-HPLC) | | | |
| Buffering | Stabiliser | | | 3 weeks | 6 weeks | | 3 weeks | 6 weeks | Osmolality |
| component | component | pH | Initial | at 35° C. | at 25° C. | Initial | at 35° C. | at 25° C. | (mOsm/kg) |
| 20 mM | 50 mM | 6.5 | 1.5 | 3.2 | 2.9 | 1.5 | 3.5 | 3.1 | 360 |
| Histidine | NaCl | | | | | | | | |
| | 50 mM | | 1.5 | 3.1 | 2.8 | 1.5 | 3.5 | 3.1 | 345 |
| | Meglumine | | | | | | | | |
| | 100 mM | | 1.5 | 2.9 | 2.7 | 1.5 | 3.2 | 2.9 | 356 |
| | Proline | | | | | | | | |
| | 200 mM | | 1.5 | 2.8 | 2.6 | 1.5 | 3.2 | 2.8 | 457 |
| | Proline | | | | | | | | |
| 20 mM | 50 mM | 6.0 | 1.2 | 2.8 | 2.5 | 1.2 | 3.1 | 2.8 | 344 |
| Histidine | NaCl | | | | | | | | |
| | 100 mM | | 1.2 | 2.8 | 2.5 | 1.3 | 3.1 | 2.8 | 459 |
| | NaCl | | | | | | | | |
| | 50 mM | | 1.2 | 2.7 | 2.4 | 1.2 | 3.0 | 2.7 | 345 |
| | Meglumine | | | | | | | | |
| | 100 mM | | 1.2 | 2.6 | 2.3 | 1.2 | 2.8 | 2.6 | 363 |
| | Proline | | | | | | | | |
| | 200 mM | | 1.2 | 2.5 | 2.2 | 1.2 | 2.7 | 2.4 | 476 |
| | Proline | | | | | | | | |
| 20 mM | 50 mM | 5.5 | 1.1 | 2.6 | 2.3 | 1.1 | 2.9 | 2.5 | 370 |
| Histidine | NaCl | | | | | | | | |
| | 100 mM | | 1.1 | 2.7 | 2.4 | 1.1 | 2.9 | 2.5 | 469 |
| | NaCl | | | | | | | | |
| | 100 mM | | 1.0 | 2.5 | 2.2 | 1.1 | NT | 2.3 | 376 |
| | Proline | | | | | | | | |
| | 200 mM | | 1.0 | 2.3 | 2.1 | 1.1 | 2.6 | 2.3 | 486 |
| | Proline | | | | | | | | |
| 20 mM | 50 mM | 5.0 | 0.9 | 2.7 | 2.1 | 0.9 | 2.9 | 2.3 | 356 |
| Histidine | NaCl | | | | | | | | |
| | 100 mM | | 0.9 | 2.8 | 2.1 | 0.9 | 2.9 | 2.3 | 449 |
| | NaCl | | | | | | | | |
| | 100 mM | | 0.8 | 2.5 | 1.9 | 0.9 | 2.8 | 2.1 | 364 |
| | Proline | | | | | | | | |
| | 200 mM | | 0.8 | 2.4 | 1.9 | 0.8 | 2.6 | 2.0 | 452 |
| | Proline | | | | | | | | |
| 20 mM | 50 mM | 5.0 | 0.9 | 3.0 | 2.5 | 0.9 | 3.2 | 2.7 | 366 |
| Glutamic | NaCl | | | | | | | | |
| acid | 100 mM | | 0.9 | 3.0 | 2.5 | 0.9 | 3.3 | 2.7 | 464 |
| | NaCl | | | | | | | | |
| | 100 mM | | 0.9 | 2.7 | 2.2 | 0.9 | 3.0 | 2.5 | 367 |
| | Proline | | | | | | | | |
| | 200 mM | | 0.9 | 2.6 | 2.1 | 0.9 | 2.8 | 2.3 | 459 |
| | Proline | | | | | | | | |
| 20 mM | 50 mM | 4.5 | 0.8 | 4.2 | 2.4 | 0.9 | 4.1 | 2.6 | 348 |
| Glutamic | NaCl | | | | | | | | |
| acid | 100 mM | | 0.8 | 3.9 | 2.5 | 0.8 | 4.6 | 2.6 | 448 |
| | NaCl | | | | | | | | |
| | 200 mM | | 0.8 | 3.7 | 2.0 | 0.8 | 4.0 | 2.2 | 466 |
| | Proline | | | | | | | | |
| | 300 mM | | 0.8 | 3.6 | 1.9 | 0.7 | 3.6 | 2.1 | 549 |
| | Proline | | | | | | | | |

NT: Not tested (due to sample injection error)

Table 2 shows that arginine combined with proline-containing formulations had the lowest percentage of HMWS when compared with other stabilisers, at both 150 and 180 mg/mL and at all pH values from 4.5 to 6.5. Additionally, formulations containing histidine as a buffer had lower HMWS than those containing glutamic acid as a buffer component.

Comparing HMWS formation, storage at 35° C. for 3 weeks showed a U-shaped trend for all formulations, with the lowest percentage of total HMWS observed at pH 5.5 and the highest percentage at pH 4.5 and 6.5. A similar trend was observed after 6 weeks at 25° C. with the lowest percentage and rate of HMWS formation observed at pH 5.0-5.5, with increasing HMWS at pH≥6.0. Although formulations at pH 4.5 had low total HMWS, the rate of HMWS formation (i.e. change from initial) was highest (Table 2).

The pH of the CSL346 formulations were within +0.1 of their target pH except the pH 4.5 formulations which ranged from 4.6-4.7; the pH of all formulations remained unchanged after storage at both temperatures.

The osmolality of formulations increased with stabiliser concentration. Proline-containing formulations had a lower osmolality than formulations containing an equivalent amount of sodium chloride. Meglumine had a similar effect on osmolality to sodium chloride (Table 2).

At the initial time point, the percentage of LMWS was comparable for all formulations ranging between 1.8 to 2.6%. After 3 weeks of storage at 35° C., the percentage of LMWS ranged from 3 to 7% irrespective of CSL346 concentration. A U-shaped trend was observed with the formulations at pH 4.5 and 6.5 having the highest LMWS, while the lowest LMWS and greatest stability was observed at ˜pH 5.5.

Predictive analyses were also performed on the formulations to determine which pH and stabiliser components gave the most favourable conformational stability (intrinsic fluorescence, first Tₘ (Tₘ1) using BCM method) and propensity to aggregate (T_{agg} at 473 nm). Formulations above pH 5 had the highest Tₘ1 and T_{agg} values, suggesting increased conformational stability and decreased propensity to aggregate above pH 5. Additionally, formulations containing proline were suggested to have a lower propensity to aggregate under thermal stress, with ˜1 to 2° C. higher T_{agg} than formulations containing sodium chloride and meglumine.

### Example 3: Stabiliser Screening

The stability of formulations at 180 mg/mL CSL346 comprising of different stabiliser components and concentrations was evaluated at pH 5.2 and 5.8 in comparison with the starting formulation at pH 6.1. Formulations assessed included arginine at different concentrations in combination with sodium chloride, proline, acetic acid, or glutamic acid, and combinations of arginine and proline or arginine and glutamic acid with an additional stabiliser such as lactic acid, isoleucine, calcium chloride or sodium chloride (refer to Table 3). Stability was assessed after 3 weeks of storage at 35° C. and 6 weeks of storage at 25° C. by SE-HPLC, CGE (R and NR), UV-Vis Spectroscopy and pH. Osmolality, SLS, intrinsic fluorescence and relative viscosity by DLS was measured at the initial time point only. All formulations screened contained arginine, 20 mM histidine, and 0.02% w/w PS80 (Table 3).

Table 3 shows that the combination of arginine and proline provide a good balance of stability by decreasing the propensity of aggregation. Arginine combined with sodium chloride formulations had higher HMWS than arginine combined with proline, acetic acid, or glutamic acid formulations. The addition of alternative additives, to either arginine with proline or to arginine with glutamate, did not enhance the stability profile. Glutamic acid and arginine in equimolar concentrations at 200 mM had the lowest propensity to form HMWS, followed by formulations containing arginine and proline, resulting in ˜1% and ˜0.5% less HMWS than the current CSL346 formulation at pH 6.1, respectively. Finally, CGE results showed that there was a suggested decrease in the purity sum of heavy and light chain under reducing conditions for all formulations at pH 5.2 after 3 weeks storage at 35° C.

TABLE 3
Stability (% HMWS by SE-HPLC) of 180 mg/mL CSL346 formulations containing 20
mM histidine, 0.02% w/w PS80 and different stabiliser components and concentrations
at pH 5.2 and 5.8. Starting formulation at pH 6.1 was also included.

| | | | | | | | | % HMWS (SE-HPLC) | | |
|---|---|---|---|---|---|---|---|---|---|---|
| | Stabiliser | Stabiliser | | | Stabiliser | | | | 3 wks | 6 wks |
| pH | Component 1 | Component 2 | | | Component 3 | | | Initial | at 35° C. | at 25° C. |
| 6.1 | 100 mM | NaCl | 50 | mM | — | — | | 2.1 | 4.2 | 3.7 |
| | Arginine | | | | | | | | | |
| 5.8 | 70 mM | Proline | 130 | mM | — | — | | 1.9 | 4.0 | 3.5 |
| | Arginine | | | | CaCl2 | 5 | mM | 1.9 | 4.0 | 3.5 |
| | | | | | | 10 | mM | 1.9 | 4.0 | 3.5 |
| | | | | | Isoleucine | 5 | mM | 2.0 | 4.0 | 3.5 |
| | | | | | | 10 | mM | 1.9 | 3.9 | 3.4 |
| | | | | | Lactic acid | 10 | mM | 1.9 | 4.0 | 3.5 |
| | | | | | | 20 | mM | 1.9 | 4.0 | 3.4 |
| | | | | | NaCl | 20 | mM | 1.9 | 4.0 | 3.5 |
| | | | | | | 50 | mM | 1.9 | 4.1 | 3.5 |
| | 100 mM | NaCl | 50 | mM | — | — | | 2.1 | 4.2 | 3.7 |
| | Arginine | Proline | 130 | mM | — | — | | 1.9 | 3.9 | 3.4 |
| | | | 175 | mm | — | — | | 1.9 | 3.8 | 3.3 |
| | | | 220 | mM | — | — | | 1.9 | 3.8 | 3.3 |
| | 130 mM | Acetic acid | 130 | mM | — | — | | 1.7 | 3.8 | 3.3 |
| | Arginine | Glutamic | 130 | mM | — | — | | 1.6 | 3.4 | 3.0 |
| | | acid | | | NaCl | 20 | mM | 1.6 | 3.4 | 3.1 |
| | | Proline | 130 | mM | — | — | | 1.9 | 3.8 | 3.3 |
| | 200 mM | Acetic acid | 200 | mM | — | — | | 1.7 | 3.5 | 3.1 |
| | Arginine | Glutamic | 200 | mM | — | — | | 1.6 | 3.1 | 2.8 |
| | | acid | | | | | | | | |
| 5.2 | 70 mM | Proline | 130 | mM | — | — | | 1.7 | 3.8 | 3.2 |
| | Arginine | | | | NaCl | 20 | mM | 1.7 | 3.8 | 3.2 |
| | | | | | | 50 | mM | 1.7 | 3.8 | 3.2 |
| | 100 mM | NaCl | 50 | mM | — | | | 1.7 | 3.9 | 3.3 |
| | Arginine | Proline | 130 | mM | — | | | 1.7 | 3.7 | 3.1 |
| | | | 175 | mM | — | | | 1.7 | 3.6 | 3.1 |
| | | | 220 | mM | — | | | 1.6 | 3.6 | 3.0 |
| | 130 mM | Glutamic | 130 | mM | — | | | 1.4 | 3.3 | 2.9 |
| | Arginine | acid | | | Proline | 130 | mM | 1.4 | 3.1 | 2.7 |
| | | Proline | 130 | mM | — | | | 1.7 | 3.6 | 3.0 |

### Example 4: pH and Stabiliser Optimisation

The pH and stabiliser concentrations of the formulation was further optimised. The stability of 180 mg/mL CSL346 formulations, all containing 20 mM histidine, 5 mM methionine and 0.02% w/w PS80, were evaluated for stabilising effects of either arginine combined with proline, arginine combined with sodium chloride, or arginine combined with glutamic acid at pH 5.2, 5.5 and 5.8, and compared with the starting formulation at pH 6.1. Excipient levels in this example were determined based on previous studies, with proline constant at 100 mM in combination with arginine ranging from 100 to 150 mM; sodium chloride constant at 50 mM with arginine constant at 100 mM; or equimolar arginine and glutamic acid both ranging from 150 mM to 200 mM. Select formulations were additionally run with no methionine present, or at 200 mg/mL CSL346. Stability was assessed by measuring SE-HPLC, CEX-HPLC, CGE (R and NR), visual description, sub-visible particle counts, UV-Vis spectroscopy, pH and osmolality after 3 months storage at 5° C., 25° C. and 35° C. All formulations comprised 20 mM histidine and 5 mM methionine with 0.02% w/w PS80.

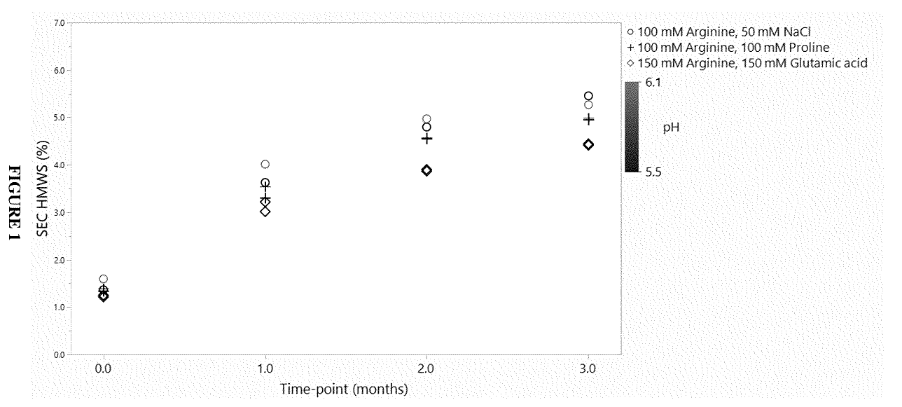

To demonstrate the difference in stability by these different combinations of excipients, the formation of HMWS over time when stored at 35° C. is shown in FIG. 1, with all formulations containing 20 mM histidine, 5 mM methionine, and 0.02% w/w PS80.

Formulations stabilised with arginine in combination with glutamic acid, while displaying low HMWS (FIG. 1) and LMWS formation, also readily formed visible particles After 3 months storage at 35° C., 50% of formulations with this combination showed visible particulates (1 to 20 spherical particles). Formulations with arginine and glutamic acid combined stored at 25° C. for 3 months experienced an additional temperature spike up to 43° C. for less than 17 hours and displayed an even greater tendency to form visible particulates, with 80% of formulations displaying a visible haze and either 1 to 20 or a flock of spherical particles. All formulations with arginine in combination with glutamic acid demonstrated significantly higher levels of sub-visible particles than those stabilised with arginine in combination with proline or sodium chloride. This led to arginine combined with glutamic acid formulations being removed from further consideration as a matrix for CSL346.

FIG. 1 shows that formulations of arginine in combination with proline had reduced percentages of HMWS when compared to arginine combined with sodium chloride. Equal or reduced formation of LMWS, and the sum percentage of heavy chain plus light chain were also observed. No visible particles were observed in arginine with proline or arginine with sodium chloride formulations. Sub-visible particles for arginine with proline formulations at all concentrations of arginine remained at equal or lower levels than the control formulation. By all these criteria, no stabilising effect was observed with increasing the arginine concentration from 100 mM up to 150 mM. Maintaining the arginine concentration at 100 mM significantly reduced the osmolality of the formulation, while providing good stability characteristics in HMWS, and LMWS following 3 months of storage at 5° C., 25° C. or 35° C.

Formulation pH was optimised within this example, for the 180 mg/mL CSL346 formulation with 125 mM arginine and 100 mM proline, by assessing the lead pH of 5.5 with 0.3 pH units either side. The original formulation of 100 mM arginine combined with 50 mM sodium chloride was assessed at pH 5.5 and at the original pH of 6.1 (Table 4). All formulations contained 20 mM histidine, 5 mM methionine and 0.02% w/w PS80.

TABLE 4
Optimisation of pH and stabiliser concentration
in CSL346 formulations

| | | 3 months | 3 months | |
|---|---|---|---|---|
| Stabiliser | | at 5° C. | at 35° C. | |
| components | pH | % HMWS | % HMWS | % LMWS |
| 125 mM Arginine, | 5.2 | 1.8 | 5.3 | 7 |
| 100 mM Proline | | | | |
| 125 mM Arginine, | 5.5 | 2.1 | 4.8 | 7 |
| 100 mM Proline | | | | |
| 125 mM Arginine, | 5.8 | 2.2 | 4.7 | 7 |
| 100 mM Proline | | | | |
| 100 mM Arginine, | 5.5 | 2.1 | 5.5 | 6 |
| 50 mM NaCl | | | | |
| 100 mM Arginine, | 6.1 | 2.7 | 5.3 | 8 |
| 50 mM NaCl | | | | |

Table 4 shows that sodium chloride-containing formulations had the highest percentage of HMWS in all conditions. Formulations of arginine with proline showed different trends at 5° C. and 35° C. (Table 4), in both cases pH 5.5 showed acceptable stability. Similar results were obtained for LMWS. No changes were observed in purity, via sum of heavy chain plus light chain for any formulation. In all cases, 180 mg/mL CSL346 in arginine and proline at pH 5.5 was equal to or more stable than the equivalent arginine and sodium chloride formulations.

Viscosity measurements were performed across a protein concentration range from 10 mg/mL to 200 mg/mL of CSL346 in 20 mM histidine, 100 mM arginine and 100 mM proline with 0.03% PS80 at pH 5.5. Viscosity across this protein concentration range varied from 1.1 to 17.2 centipoise (cP), with the viscosity remaining under 15 cP for concentrations up to 190 mg/ml. At 160 mg/mL, the viscosity was 8.0 cP and at 170 mg/mL the viscosity was <11 cP.

### Example 4: Antioxidant

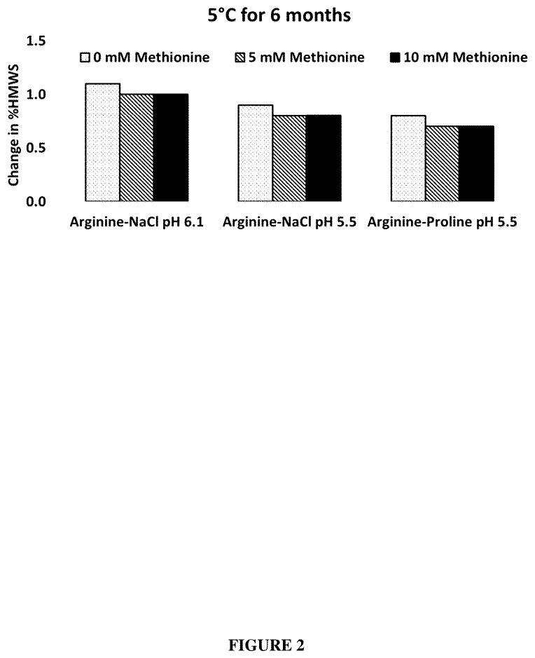

To further optimise the CSL346 formulation, the effect of methionine as a potential antioxidant, was assessed. The stability of formulations at 150 mg/mL CSL346 containing arginine combined with sodium chloride or proline, with or without methionine was assessed after 6 months of storage at 5° C. All formulations included 20 mM histidine and 0.02% w/w PS80. After 6 months of storage at 5° C., no differences were observed in LMWS (2%), sum of heavy and light chains (98%) between formulations with and without methionine. A minor decrease in the percentage of HMWS formation was observed in formulations containing methionine of ˜0.1% less than formulations without methionine (FIG. 2).

### Example 5: Surfactant Optimisation

To optimise the CSL346 formulation surfactant concentration, the effects of PS80 were assessed in relation to protection against particulate formation induced by thermal stress after storage at 35° C. for 4 weeks. Five levels of PS80 was assessed from 0 to 0.06% w/w in 170 mg/mL CSL346 formulations containing 20 mM histidine and arginine combined with sodium chloride, proline or glutamic acid. See Table 5 for further details.

TABLE 5
Surfactant study formulations

| Buffer | Stabiliser 1 | Stabiliser 2 | Antioxidant | pH |
|---|---|---|---|---|
| 20 mM | 100 mM Arginine | 50 mM NaCl | — | 6.1 |
| Histidine | 125 mM Arginine | 100 mM Proline | — | 5.5 |
| | 180 mM Arginine | 180 mM Glutamic | 5 mM | |
| | | acid | Methionine | |

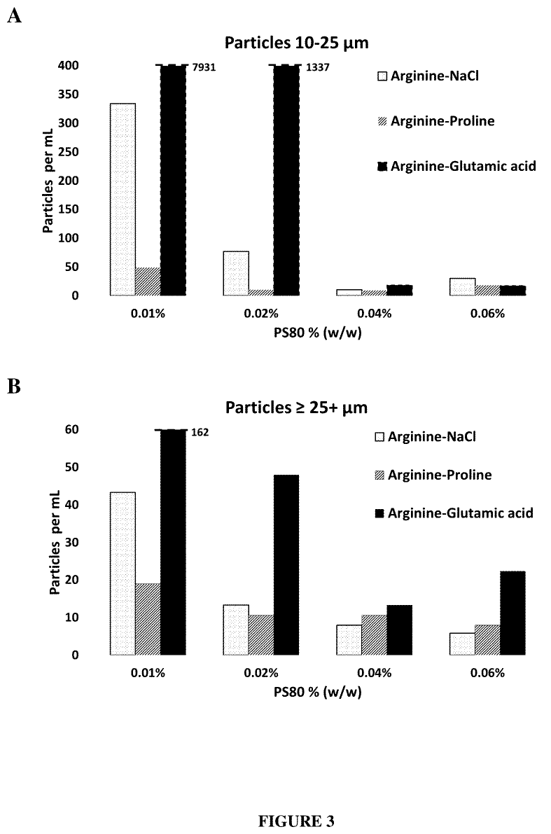

Visible particles were observed in the 0% PS80 formulation, with adequate protection provided by 0.01% w/w PS80 against formation of visible particles in all formulations. It was observed that increased concentrations of PS80 provided better protection against sub-visible particulate formation in all formulations. Formulations containing arginine combined with proline at pH 5.5, displayed the lowest sub-visible particle counts across most levels of PS80, while formulations containing arginine with glutamic acid at pH 5.5 or arginine with sodium chloride at pH 6.1, showed significantly higher sub-visible particle counts at PS80 levels<0.04% w/w (FIG. 3). In addition, the 0.01% PS80 formulation containing arginine with glutamic acid showed increased turbidity during visual description after storage for 4 weeks at 35° C. Based on this data, the PS80 level of the formulation containing arginine combined with proline at pH 5.5 was optimised to 0.03% w/w to improve robustness against sub-visible and visible particle formation over time and under stirring induced stress.

### Example 7: Pre-Filled Syringe Intrinsic Components Evaluation

The following example evaluated the suitability for CSL346 DP storage in pre-filled syringes (PFS). The stability of CSL346 in the presence of silicone oil (Dow Corning® 360 Medical fluid) and tungsten, intrinsic components of PFS, was evaluated after 4 weeks of storage under thermal stress at 35° C.

Two concentrations of silicone oil (0.5 and 1.0 mg/mL) and one concentration of tungsten (500 ppb) were evaluated in formulations of 170 mg/mL CSL346, with 20 mM histidine, 0.02% PS80 at pH 5.5 and 6.1, see Table 6 for further details.

TABLE 6
PFS intrinsic components study formulations

| Buffer | Stabiliser 1 | Stabiliser 2 | Antioxidant | pH |
|---|---|---|---|---|
| 20 mM | 100 mM Arginine | 50 mM NaCl | — | 6.1 |
| Histidine | 125 mM Arginine | 100 mM Proline | — | 5.5 |
| | 180 mM Arginine | 180 mM Glutamic | 5 mM | |
| | | acid | Methionine | |

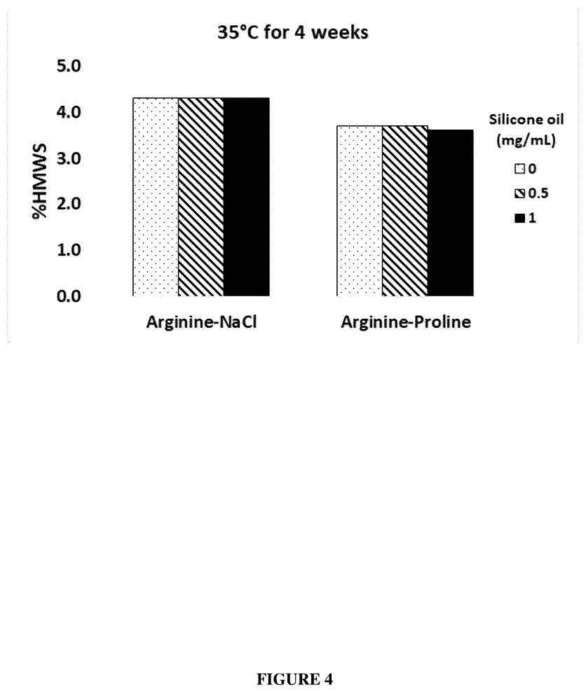

As demonstrated in FIG. 4, silicone oil was not observed to have any effect on stability after 4 weeks of storage at 35° C., with HMWS, LMWS, sum of heavy and light chains remaining within assay variation between formulations with and without silicone oil. No visible particles were observed in formulations with and without silicone oil.

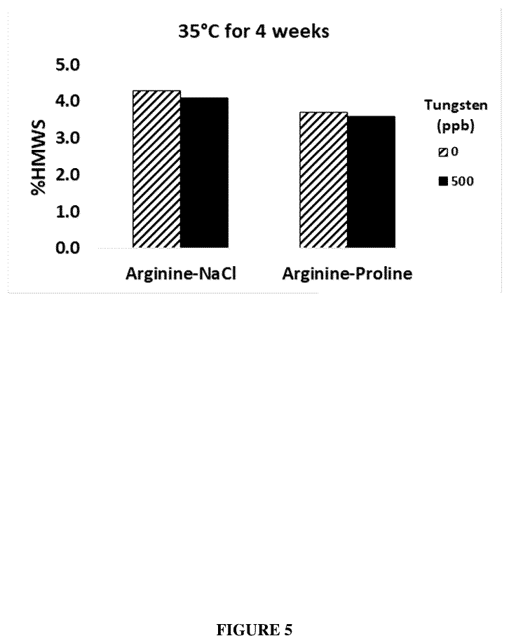

As demonstrated in FIG. 5, tungsten did not have any effect on stability after 4 weeks of storage at 35° C., with HMWS, LMWS and sum of heavy and light chains remaining within assay variation between formulations with and without tungsten. No difference between the visual appearance of formulations with and without tungsten was observed.

### Example 8: Exemplary Formulation

An exemplary antibody formulation, based on the results described above, is shown in Table 7.

TABLE 7
Exemplary antibody formulation

| Component | Amount or concentration | |
|---|---|---|
| Antibody | 160 | mg/mL |
| L-Histidine | 20 | mM |
| L-Arginine | 100 | mM |
| L-Proline | 100 | mM |
| Polysorbate 80 | 0.03% | w/v |

| | |
|---|---|
| pH | 5.5 (5.2-5.8) |
| Viscosity | 7.6 mPa*s (@ 20° C.) |

While the concentration of antibody exemplified in Table 7 is 160 mg/mL with 0.03% w/w PS80, the formulation is also suited to concentrations of antibody up to at least 200 mg/mL and from 0.02-0.04% w/w PS80.

### Example 9: Long-Term Stability of Exemplary Formulation

The long-term stability of the exemplary CSL346 formulation described in Example 8 was assessed in glass vials by holding the formulation for 0, 1, 3, 6, 9 and 12 months at 5° C. (±3° C.), or under stress at 25° C. (±2° C.) or 35° C. (±2° C.). The results are shown in Tables 8, 9 and 10.

CSL346 was shown to have improved long-term stability in the exemplary formulation when compared to another IgG4 antibody formulated in a similar formulation. In particular, less accumulation of LMWS for CSL346 at 6 months under stress at 25° C. (±2° C.) and at 35° C. (±2° C.) was observed.

### Example 10: Long-Term Stability of a Further Exemplary Formulation

The long-term stability of an exemplary CSL346 formulation, with 180 mg/mL CSL346 in 20 mM histidine, 100 mM arginine, 100 mM proline, 0.02% PS80 at pH 5.5, was assessed in vials by holding the formulation for 1, 3 and 6 months at 5° C. (±3° C.), or under stress at 25° C. (±2° C.) or 35° C. (±2° C.). The results are shown in Tables 11-13.

### Example 11: Long-Term Stability of Exemplary Formulations in Pre-Filled Syringes

The long-term stability of the exemplary CSL346 formulation described in Example 8 and formulations comprising 100 mg/mL and 50 mg/mL CSL346 was assessed in pre-filled syringes by holding the formulation for 0, 1 and 3 months at 5° C. (±3° C.), or under stress at 25° C. (±2° C.) or 35° C. (±2° C.). The results are shown in Tables 14 to 22.

The stability of 160 mg/ml, 100 mg/ml and 50 mg/ml CSL346 in saline placebo was also assessed in pre-filled syringes (PFS) by holding the formulation for 0 and 3 months at 5° C. (±3° C.), or under stress at 25° C. (±2° C.) or 35° C. (±2° C.). At 0 months, the pH was 5.6 and at 3 months pH5.1-5.2 at all conditions. No visible particles were observed at any time point, or under any condition. Sub visible particles were determined with 1, 98, 57 and 183 sub visible particles ≥10 μm per container were observed at 0 months and 3 months at 5° C. (±3° C.), 25° C. (±2° C.) or 35° C. (±2° C.) respectively. No sub visible particles of ≥25 μm per container were observed at any time point or storage condition

TABLE 8
Long-term stability of exemplary CSL346 formulation (160 mg/mL) in glass vials after 12 months at 5° C. ± 3° C.

| | | Provisional | | | | | | |
|---|---|---|---|---|---|---|---|---|
| | | acceptance | Number of months | | | | | |
| Test | Units | criteria | 0 | 1 | 3 | 6 | 9 | 12 |
| Protein content | mg/ml | 150-170 mg/ml | 159 | 160 | 160 | 160 | 160 | 159 |
| by UV spectropho- | | (±10% from | | | | | | |
| tometry (A280 nm) | | initial) | | | | | | |
| pH | pH units | 5.2-5.8 | 5.5 | 5.4 | 5.4 | 5.5 | 5.4 | 5.4 |
| Visible particles | N/A | No visible | Pass | Pass | Pass | Pass | Pass | Pass |
| (description) | | particles | | | | | | |
| Degree of | BY scale | Pass if equal or | Pass | Pass | Pass | Pass | Pass | Pass |
| colouration | | less intensely | ≤BY2 to | ≤BY2 to | ≤BY2 to | ≤BY2 to | ≤BY2 to | <BY2 to |
| of liquids | | coloured than BY1 | ≥BY3 | ≥BY3 | ≥BY3 | ≥BY3 | ≥BY3 | ≥BY3 |
| | | (≤BY1) to equal | | | | | | |
| | | or more intensely | | | | | | |
| | | coloured than BY4 | | | | | | |
| | | (≥BY4) | | | | | | |
| Clarity | NTU | Pass if ≤30 NTU | 12 | 11 | 10 | 9 | 11 | 10 |
| Container Closure | N/A | Pass if all vials | Pass | NS | NS | Pass | NS | Pass |
| Integrity Testing | | integral | | | | | | |
| Surrogate potency | Ratio | 60-150% relative | 105 | 109 | 96 | 101 | 109 | 110 |
| for VEGF-B | | to CSL346 reference | | | | | | |
| binding by | | standard | | | | | | |
| ELISA | | | | | | | | |
| Endotoxin | EU/mL | ≤8.00 EU/mL | <5.00 | NS | NS | NS | NS | NS |
| SE-HPLC | % | ≤5% HMWS | 1 | 1 | 2 | 2 | 3 | 3 |
| | | ≥95% monomer | 99 | 98 | 98 | 98 | 97 | 97 |
| CE-SDS (Non- | % | ≤10% LMWS | 1 | 2 | 2 | 1 | 2 | 2 |
| Reducing) | | ≥90% main peak | 98 | 98 | 97 | 97 | 96 | 96 |
| CE-SDS (Reducing) | % | ≥95% sum of | 98 | 98 | 98 | 98 | 97 | 97 |
| | | heavy chain + | | | | | | |
| | | light chain | | | | | | |
| Sub-visible | Average | ≤6000 particles | 30 | 35 | 23 | 37 | 17 | 19 |
| particle | cumulative | of ≥10 μm per | | | | | | |
| count | counts/2 mL | container | | | | | | |
| | container | ≤600 particles | | | | | | |
| | | of ≥25 μm per | 0 | 3 | 2 | 4 | 0 | 1 |
| | | container | | | | | | |
| Osmolality | mOsmo/kg | 330-430 mOsm/kg | 368 | 375 | 374 | NS | NS | 365 |

N/A—Not Applicable;
NS—Not Scheduled

TABLE 9
Long-term stability of exemplary CSL346 formulation (160 mg/mL) in glass vials after 12 months at 25° C. ± 2° C.

| | | Provisional | | | | | | |
|---|---|---|---|---|---|---|---|---|
| | | acceptance | Number of months | | | | | |
| Test | Units | criteria | 0 | 1 | 3 | 6* | 9 | 12 |
| Protein content | mg/ml | 150-170 mg/ml | 159 | 160 | 160 | 160 | 160 | 160 |
| by UV spectropho- | | (±10% from | | | | | | |
| tometry (A280 nm) | | initial) | | | | | | |
| pH | pH units | 5.2-5.8 | 5.5 | 5.4 | 5.5 | 5.5 | 5.4 | 5.5 |
| Visible particles | N/A | No visible | Pass | Pass | Pass | Pass | Pass | Pass |
| (description) | | particles | | | | | | |
| Degree of | BY scale | Pass if equal or | ≤BY2 to | ≤BY2 to | ≤BY2 to | <BY2 to | ≤BY2 to | ≤BY2 to |
| colouration | | less intensely | ≥BY3 | ≥BY3 | ≥BY3 | ≥BY3 | ≥BY3 | ≥BY3 |
| of liquids | | coloured than BY1 | | | | | | |
| | | (≤BY1) to equal | | | | | | |
| | | or more intensely | | | | | | |
| | | coloured than BY4 | | | | | | |
| | | (≥BY4) | | | | | | |
| Clarity | NTU | Pass if ≤30 NTU | 12 | 11 | 10 | 11 | 11 | 11 |
| Container Closure | N/A | Pass if all vials | Pass | NS | NS | Pass | NS | Pass |
| Integrity Testing | | integral | | | | | | |
| Surrogate potency | Ratio | 60-150% | 105 | 107 | 113 | 95 | 101 | 136 |
| for VEGF-B | | | | | | | | |
| binding by | | | | | | | | |
| ELISA | | | | | | | | |
| Endotoxin | EU/mL | ≤8.00 EU/mL | <5.00 | NS | NS | NS | NS | <0.18 |
| SE-HPLC | % | ≤5% HMWS | 1 | 2 | 3 | 5 | 5 | 4 |
| | | ≥95% monomer | 99 | 97 | 97 | 95 | 95 | 95 |
| CE-SDS (Non- | % | ≤10% LMWS | 1 | 2 | 3 | 4 | 5 | 6 |
| Reducing) | | ≥90% main peak | 98 | 97 | 95 | 93 | 92 | 91 |
| CE-SDS | % | ≥95% sum of | 98 | 98 | 97 | 97 | 95 | 95 |
| (Reducing) | | heavy chain + | | | | | | |
| | | light chain | | | | | | |
| Sub-visible | Average | ≤6000 particles | 30 | 34 | 7 | 38 | 40 | 67 |
| particle count | cumulative | of ≥10 μm per | | | | | | |
| | counts/2 mL | container | | | | | | |
| | container | ≤600 particles | | | | | | |
| | | of ≥25 μm per | 0 | 2 | 0 | 1 | 1 | 1 |
| | | container | | | | | | |
| Osmolality | mOsmo/kg | 330-430 mOsm/kg | 368 | 376 | 374 | NS | NS | 373 |

N/A—Not Applicable;
NS—Not Scheduled;
*Prior to the six month time point material was out of specification for 2 hours with a temperature excursion to 58° C. occurring.

TABLE 10
Long-term stability of exemplary CSL346 formulation (160 mg/mL) in glass vials after 12 months at 35° C. ± 2° C.

| | | Provisional | | | | | | |
|---|---|---|---|---|---|---|---|---|
| | | acceptance | Number of months | | | | | |
| Test | Units | criteria | 0 | 1 | 3 | 6 | 9 | 12 |
| Protein content | mg/ml | 150-170 mg/ml | 159 | 160 | 160 | 160 | 159 | 161 |
| by UV spectropho- | | (±10% from | | | | | | |
| tometry (A280 nm) | | initial) | | | | | | |
| pH | pH units | 5.2-5.8 | 5.5 | 5.4 | 5.5 | 5.5 | 5.4 | 5.5 |
| Visible particles | N/A | No visible | Pass | Pass | Pass | Pass | Pass | Pass |
| (description) | | particles | | | | | | |
| Degree of | BY scale | Pass if equal | ≤BY2 to | ≤BY2 to | ≤BY2 to | ≤BY2 to | <BY2 to | ≤BY2 to |
| colouration | | or less intensely | ≥BY3 | ≥BY3 | ≥BY3 | ≥BY3 | ≥BY3 | ≥BY3 |
| of liquids | | coloured than BY1 | | | | | | |
| | | (≤BY1) to equal | | | | | | |
| | | or more intensely | | | | | | |
| | | coloured than BY4 | | | | | | |
| | | (≥BY4) | | | | | | |
| Clarity | NTU | Pass if ≤30 NTU | 12 | 11 | 11 | 12 | 16 | 14 |
| Container Closure | N/A | Pass if all | Pass | NS | NS | Pass | NS | Pass |
| Integrity Testing | | vials integral | | | | | | |
| Surrogate potency | Ratio | 60-150% | 105 | 115 | 92 | 95 | 98 | 106 |
| for VEGF-B | | | | | | | | |
| binding by | | | | | | | | |
| ELISA | | | | | | | | |
| Endotoxin | EU/mL | ≤8.00 EU/mL | <5.00 | NS | NS | NS | NS | <0.18 |
| SE-HPLC | % | ≤5% HMWS | 1 | 3 | 5 | 7 | 8 | 8 |
| | | ≥95% monomer | 99 | 96 | 95 | 92 | 91 | 90 |
| CE-SDS (Non- | % | ≤10% LMWS | 1 | 3 | 6 | 11 | 15 | 18 |
| Reducing) | | ≥90% main peak | 98 | 95 | 91 | 86 | 82 | 78 |
| CE-SDS | % | ≥95% sum of | 98 | 97 | 95 | 93 | 89 | 87 |
| (Reducing) | | heavy chain + | | | | | | |
| | | light chain | | | | | | |
| Sub-visible | Average | ≤6000 particles | 30 | 34 | 562 | 267 | 12142 | 17169 |
| particle | cumulative | of ≥10 μm per | | | | | | |
| count | counts/2 mL | container | | | | | | |
| | container | ≤600 particles | 0 | 1 | 5 | 6 | 30 | 32 |
| | | of ≥25 μm per | | | | | | |
| | | container | | | | | | |
| Osmolality | mOsmo/kg | 330-430 mOsm/kg | 368 | 379 | 373 | 384 | NS | 379 |

N/A—Not Applicable;
NS—Not Scheduled

TABLE 11
Long-term stability of exemplary CSL346 formulation (180 mg/mL)
after 6 months at 5° C. ± 3° C.

| | | Provisional | | | |
|---|---|---|---|---|---|
| | | acceptance | Number of months | | |
| Test | Units | criteria | 0 | 3 | 6 |
| CE-SDS | % peak | ≤10% LMWS | 2 | 2 | 2 |
| (Non-Reducing) | area | ≥85% main peak | 98 | 97 | 98 |
| CE-SDS | % peak | ≥95% sum of | 98 | 98 | 98 |
| (Reducing) | area | heavy chain + | | | |
| | | light chain | | | |
| Description | N/A | Pass if opalescent | Pass | Pass | Pass |
| | | to clear, brown- | | | |
| | | yellow to colourless | | | |
| | | liquid. No visible | | | |
| | | particles. | | | |
| pH | pH units | 5.2-5.8 | 5.5 | 5.5 | 5.5 |
| Protein concen- | mg/mL | +/− 10% of | 180 | S | 180 |
| tration by UV | | Initial Value or | | | |
| spectropho- | | 170 to 190 mg/mL? | | | |
| tometry (A280) | | | | | |
| SE-HPLC | % peak | ≤5% HMWS | 1.4 | 2.3 | 1.4 |
| | area | ≥95% monomer | 98.5 | 97.5 | 98.5 |

N/A—Not Applicable;
NS—Not Scheduled;
S—Scheduled

TABLE 12
Long-term stability of exemplary CSL346 formulation (180 mg/mL)
after 6 months at 25° C. ± 2° C.

| | | Provisional | | | | |
|---|---|---|---|---|---|---|
| | | acceptance | Number of months | | | |
| Test | Units | criteria | 0 | 2 | 3 | 6 |
| CE-SDS | % peak | ≤10% LMWS | 2 | 3 | 4 | 5 |
| (Non-Reducing) | area | ≥85% main peak | 98 | 96 | 95 | 93 |
| CE-SDS | % peak | ≥95% sum of | 98 | 97 | 97 | 96 |
| (Reducing) | area | heavy chain + | | | | |
| | | light chain | | | | |
| Description | N/A | Pass if opalescent | Pass | NS | Pass | Pass |
| | | to clear, brown- | | | | |
| | | yellow to colourless | | | | |
| | | liquid. No visible | | | | |
| | | particles. | | | | |
| pH | pH units | 5.2-5.8 | 5.5 | 5.5 | 5.5 | 5.5 |
| Protein concen- | mg/mL | +/− 10% of | 180 | 185 | S | 181 |
| tration by UV | | Initial Value or | | | | |
| spectropho- | | 170 to 190 mg/mL? | | | | |
| tometry (A280) | | | | | | |
| SE-HPLC | % peak | ≤5% HMWS | 1.4 | 3.7 | 3.7 | 4.4 |
| | area | ≥95% monomer | 98.5 | 95.7 | 95.9 | 95.3 |

N/A—Not Applicable;
NS—Not Scheduled;
S—Scheduled

TABLE 13
Long-term stability of exemplary CSL346 formulation (180 mg/mL)
after 3 months at 35° C. ± 2° C.

| | | Provisional | | | | |
|---|---|---|---|---|---|---|
| | | acceptance | Number of months | | | |
| Test | Units | criteria | 0 | 1 | 2 | 3 |
| CE-SDS | % peak | ≤10% LMWS | 2 | 3 | 5 | 6 |
| (Non-Reducing) | area | ≥85% main peak | 98 | 95 | 94 | 92 |
| CE-SDS | % peak | ≥95% sum of | 98 | 97 | 96 | 96 |
| (Reducing) | area | heavy chain + | | | | |
| | | light chain | | | | |
| Description | N/A | Pass if opalescent | Pass | NS | NS | Pass |
| | | to clear, brown- | | | | |
| | | yellow to colourless | | | | |
| | | liquid. No visible | | | | |
| | | particles. | | | | |
| pH | pH units | 5.2-5.8 | 5.5 | 5.6 | 5.6 | 5.5 |
| Protein concen- | mg/mL | +/− 10% of | 180 | 181 | 187 | S |
| tration by UV | | Initial Value or | | | | |
| spectropho- | | 170 to 190 mg/mL? | | | | |
| tometry (A280) | | | | | | |
| SE-HPLC | % peak | ≤5% HMWS | 1.4 | 3.4 | 4.8 | 5.3 |
| | area | ≥95% monomer | 98.5 | 96.4 | 94.0 | 94.1 |

N/A—Not Applicable;
NS—Not Scheduled;
S—Scheduled

TABLE 14
Long-term stability of exemplary CSL346 formulation (160 mg/mL)
in PFS after 3 months at 5° C. ± 3° C.

| | | Provisional | | | |
|---|---|---|---|---|---|
| | | acceptance | Number of months | | |
| Test | Units | criteria | 0 | 1 | 3 |
| Protein content | mg/ml | 150-170 mg/ml | 163 | 158 | 157 |
| by UV spectro- | | (±10% from | | | |
| photometry | | initial) | | | |
| (A280 nm) | | | | | |
| pH | pH units | 5.2-5.8 | 5.6 | 5.5 | 5.6 |
| Visible | N/A | No visible | Pass | Pass | Pass |
| particles | | particles | | | |
| (description) | | | | | |
| SE-HPLC | % | ≤5% HMWS | 0.9 | 1.2 | 1.5 |
| | | ≥95% monomer | 98.9 | 98.6 | 98.3 |
| CE-SDS | % | ≤10% LMWS | 1 | 2 | 2 |
| (Non-Reducing) | | ≥90% main peak | 99 | 98 | 98 |
| CE-SDS | % | ≥95% sum of | 98 | 98 | 98 |
| (Reducing) | | heavy chain + | | | |
| | | light chain | | | |
| Sub-visible | Average | ≤6000 particles | 23 | NS | NS |
| particle count | cumulative | of ≥10 μm per | | | |
| | counts/2 mL | container | | | |
| | container | ≤600 particles | 1 | | |
| | | of ≥25 μm per | | | |
| | | container | | | |

N/A—Not Applicable;
NS—Not Scheduled

TABLE 15
Long-term stability of exemplary CSL346 formulation (160 mg/mL) in
PFS after 3 months at 25° C. ± 3° C.

| | | Provisional | | | |
|---|---|---|---|---|---|
| | | acceptance | Number of months | | |
| Test | Units | criteria | 0 | 1 | 3 |
| Protein content | mg/ml | 150-170 mg/ml | 163 | 159 | 159 |
| by UV spectro- | | (±10% from | | | |
| photometry | | initial) | | | |
| (A280 nm) | | | | | |
| pH | pH units | 5.2-5.8 | 5.6 | 5.6 | 5.6 |
| Visible | N/A | No visible | Pass | Pass | Pass |
| particles | | particles | | | |
| (description) | | | | | |
| SE-HPLC | % | ≤5% HMWS | 0.9 | 1.9 | 2.4 |
| | | ≥95% monomer | 98.9 | 98.0 | 97.4 |
| CE-SDS | % | ≤10% LMWS | 1 | 2 | 3 |
| (Non-Reducing) | | ≥90% main peak | 99 | 97 | 97 |
| CE-SDS | % | ≥95% sum of | 98 | 98 | 98 |
| (Reducing) | | heavy chain + | | | |
| | | light chain | | | |
| Sub-visible | Average | ≤6000 particles | 23 | NS | NS |
| particle count | cumulative | of ≥10 μm per | | | |
| | counts/2 mL | container | | | |
| | container | ≤600 particles | 1 | | |
| | | of ≥25 μm per | | | |
| | | container | | | |

N/A—Not Applicable;
NS—Not Scheduled

TABLE 16
Long-term stability of exemplary CSL346 formulation (160 mg/mL) in
PFS after 3 months at 35° C. ± 3° C.

| | | Provisional | | | |
|---|---|---|---|---|---|
| | | acceptance | Number of months | | |
| Test | Units | criteria | 0 | 1 | 3 |
| Protein content | mg/ml | 150-170 mg/ml | 163 | 160 | 160 |
| by UV spectro- | | (±10% from | | | |
| photometry | | initial) | | | |
| (A280 nm) | | | | | |
| pH | pH units | 5.2-5.8 | 5.6 | 5.6 | 5.6 |
| Visible | N/A | No visible | Pass | Pass | Pass |
| particles | | particles | | | |
| (description) | | | | | |
| SE-HPLC | % | ≤5% HMWS | 0.9 | 2.6 | 3.7 |
| | | ≥95% monomer | 98.9 | 97.1 | 95.9 |
| CE-SDS | % | ≤10% LMWS | 1 | 3 | 6 |
| (Non- | | ≥90% main peak | 99 | 96 | 93 |
| Reducing) | | | | | |
| CE-SDS | % | ≥95% sum of | 98 | 98 | 98 |
| (Reducing) | | heavy chain + | | | |
| | | light chain | | | |
| Sub-visible | Average | ≤6000 particles | 23 | NS | 1597 |
| particle count | cumulative | of ≥10 μm per | | | |
| | counts/2 mL | container | | | |
| | container | ≤600 particles | 1 | | 191 |
| | | of ≥25 μm per | | | |
| | | container | | | |

N/A—Not Applicable;
NS—Not Scheduled

TABLE 17
Long-term stability of exemplary CSL346 formulation (100 mg/mL)
in PFS after 3 months at 5° C. ± 3° C.

| | | Provisional | | | |
|---|---|---|---|---|---|
| | | acceptance | Number of months | | |
| Test | Units | criteria | 0 | 1 | 3 |
| Protein content | mg/ml | | 101 | 102 | 103 |
| by UV spectro- | | | | | |
| photometry | | | | | |
| (A280 nm) | | | | | |
| pH | pH units | 5.2-5.8 | 5.5 | 5.5 | 5.6 |
| Visible | N/A | No visible | Pass | Pass | Pass |
| particles | | particles | | | |
| (description) | | | | | |
| SE-HPLC | % | ≤5% HMWS | 0.8 | 1 | 1.3 |
| | | ≥95% monomer | 98.9 | 98.8 | 98.5 |
| CE-SDS | % | ≤10% LMWS | 1 | 2 | 2 |
| (Non-Reducing) | | ≥90% main peak | 99 | 98 | 98 |
| CE-SDS | % | ≥95% sum of | 98 | 98 | 98 |
| (Reducing) | | heavy chain + | | | |
| | | light chain | | | |
| Sub-visible | Average | ≤6000 particles | 11 | NS | NS |
| particle count | cumulative | of ≥10 μm per | | | |
| | counts/2 mL | container | | | |
| | container | ≤600 particles | 1 | | |
| | | of ≥25 μm per | | | |
| | | container | | | |

N/A—Not Applicable;
NS—Not Scheduled

TABLE 18
Long-term stability of exemplary CSL346 formulation (100 mg/mL) in
PFS after 3 months at 25° C. ± 3° C.

| | | Provisional | | | |
|---|---|---|---|---|---|
| | | acceptance | Number of months | | |
| Test | Units | criteria | 0 | 1 | 3 |
| Protein content | mg/ml | | 101 | 102 | 100 |
| by UV spectro- | | | | | |
| photometry | | | | | |
| (A280 nm) | | | | | |
| pH | pH units | 5.2-5.8 | 5.5 | 5.5 | 5.6 |
| Visible | N/A | No visible | Pass | Pass | Pass |
| particles | | particles | | | |
| (description) | | | | | |
| SE-HPLC | % | ≤5% HMWS | 0.8 | 1.5 | 2 |
| | | ≥95% monomer | 98.9 | 98.2 | 97.8 |
| CE-SDS | % | ≤10% LMWS | 1 | 2 | 3 |
| (Non-Reducing) | | ≥90% main peak | 99 | 97 | 97 |
| CE-SDS | % | ≥95% sum of | 98 | 98 | 98 |
| (Reducing) | | heavy chain + | | | |
| | | light chain | | | |
| Sub-visible | Average | ≤6000 particles | 11 | NS | NS |
| particle count | cumulative | of ≥10 μm per | | | |
| | counts/2 mL | container | | | |
| | container | ≤600 particles | 1 | | |
| | | of ≥25 μm per | | | |
| | | container | | | |

N/A—Not Applicable;
NS—Not Scheduled

TABLE 19
Long-term stability of exemplary CSL346 formulation (100 mg/mL) in
PFS after 3 months at 35° C. ± 3° C.

| | | Provisional | | | |
|---|---|---|---|---|---|
| | | acceptance | Number of months | | |
| Test | Units | criteria | 0 | 1 | 3 |
| Protein content | mg/ml | | 101 | 107 | 112 |
| by UV spectro- | | | | | |
| photometry | | | | | |
| (A280 nm) | | | | | |
| pH | pH units | 5.2-5.8 | 5.5 | 5.5 | 5.6 |
| Visible | N/A | No visible | Pass | Pass | Pass |
| particles | | particles | | | |
| (description) | | | | | |
| SE-HPLC | % | ≤5% HMWS | 0.8 | 2.1 | 3 |
| | | ≥95% monomer | 98.9 | 97.6 | 96.6 |
| CE-SDS | % | ≤10% LMWS | 1 | 3 | 6 |
| (Non-Reducing) | | ≥90% main peak | 99 | 96 | 94 |
| CE-SDS | % | ≥95% sum of | 98 | 98 | 96 |
| (Reducing) | | heavy chain + | | | |
| | | light chain | | | |
| Sub-visible | Average | ≤6000 particles | 11 | NS | NS |
| particle count | cumulative | of ≥10 μm per | | | |
| | counts/2 mL | container | | | |
| | container | ≤600 particles | 1 | | |
| | | of ≥25 μm per | | | |
| | | container | | | |

N/A—Not Applicable;
NS—Not Scheduled

TABLE 20
Long-term stability of exemplary CSL346 formulation (50 mg/mL) in
PFS after 3 months at 5° C. ± 3° C.

| | | Provisional | | | |
|---|---|---|---|---|---|
| | | acceptance | Number of months | | |
| Test | Units | criteria | 0 | 1 | 3 |
| Protein content | mg/ml | | 51 | 53 | 53 |
| by UV spectro- | | | | | |
| photometry | | | | | |
| (A280 nm) | | | | | |
| pH | pH units | 5.2-5.8 | 5.5 | 5.5 | 5.6 |
| Visible | N/A | No visible | Pass | Pass | Pass |
| particles | | particles | | | |
| (description) | | | | | |
| SE-HPLC | % | ≤5% HMWS | 0.7 | 0.9 | 1.1 |
| | | ≥95% monomer | 99 | 98.9 | 98.7 |
| CE-SDS | % | ≤10% LMWS | 1 | 2 | 2 |
| (Non-Reducing) | | ≥90% main peak | 99 | 98 | 98 |
| CE-SDS | % | ≥95% sum of | 98 | 98 | 98 |
| (Reducing) | | heavy chain + | | | |
| | | light chain | | | |
| Sub-visible | Average | ≤6000 particles | 85 | NS | NS |
| particle count | cumulative | of ≥10 μm per | | | |
| | counts/2 mL | container | | | |
| | container | ≤600 particles | 1 | | |
| | | of ≥25 μm per | | | |
| | | container | | | |

N/A—Not Applicable;
NS—Not Scheduled

TABLE 21
Long-term stability of exemplary CSL346 formulation (50 mg/mL)
in PFS after 3 months at 25° C. ± 3° C.

| | | Provisional | | | |
|---|---|---|---|---|---|
| | | acceptance | Number of months | | |
| Test | Units | criteria | 0 | 1 | 3 |
| Protein content | mg/ml | | 51 | 54 | 56 |
| by UV spectro- | | | | | |
| photometry | | | | | |
| (A280 nm) | | | | | |
| pH | pH units | 5.2-5.8 | 5.5 | 5.5 | 5.6 |
| Visible | N/A | No visible | Pass | Pass | Pass |
| particles | | particles | | | |
| (description) | | | | | |
| SE-HPLC | % | ≤5% HMWS | 0.7 | 1.2 | 1.5 |
| | | ≥95% monomer | 99 | 98.6 | 98.3 |
| CE-SDS | % | ≤10% LMWS | 1 | 2 | 3 |
| (Non-Reducing) | | ≥90% main peak | 99 | 98 | 97 |
| CE-SDS | % | ≥95% sum of | 98 | 98 | 98 |
| (Reducing) | | heavy chain + | | | |
| | | light chain | | | |
| Sub-visible | Average | ≤6000 particles | 85 | NS | NS |
| particle count | cumulative | of ≥10 μm per | | | |
| | counts/2 mL | container | | | |
| | container | ≤600 particles | 1 | | |
| | | of ≥25 μm per | | | |
| | | container | | | |

N/A—Not Applicable;
NS—Not Scheduled

TABLE 22
Long-term stability of exemplary CSL346 formulation (50 mg/mL)
in PFS after 3 months at 35° C. ± 3° C.

| | | Provisional | | | |
|---|---|---|---|---|---|
| | | acceptance | Number of months | | |
| Test | Units | criteria | 0 | 1 | 3 |
| Protein content | mg/ml | | 51 | 52 | 52 |
| by UV spectro- | | | | | |
| photometry | | | | | |
| (A280 nm) | | | | | |
| pH | pH units | 5.2-5.8 | 5.5 | 5.5 | 5.6 |
| Visible | N/A | No visible | Pass | Pass | Pass |
| particles | | particles | | | |
| (description) | | | | | |
| SE-HPLC | % | ≤5% HMWS | 0.7 | 1.5 | 2.2 |
| | | ≥95% monomer | 99 | 98.1 | 97.3 |
| CE-SDS | % | ≤10% LMWS | 1 | 3 | 5 |
| (Non-Reducing) | | ≥90% main peak | 99 | 97 | 94 |
| CE-SDS | % | ≥95% sum of | 98 | 98 | 96 |
| (Reducing) | | heavy chain + | | | |
| | | light chain | | | |
| Sub-visible | Average | ≤6000 particles | 85 | NS | NS |
| particle count | cumulative | of ≥10 μm per | | | |
| | counts/2 mL | container | | | |
| | container | ≤600 particles | 1 | | |
| | | of ≥25 μm per | | | |
| | | container | | | |

N/A—Not Applicable;
NS—Not Scheduled

1. A liquid pharmaceutical formulation comprising a protein comprising an antigen binding domain that binds to or specifically binds to vascular endothelial growth factor B (VEGF-B), an organic acid buffer, a non-ionic surfactant and at least one amino acid stabiliser, wherein the formulation has a pH of 5.0 to 6.0.

2. The formulation of claim 1, wherein the protein is present in the formulation at a concentration of at least 50 mg/mL, or at least 100 mg/mL, or at least 130 mg/mL, or at least 150 mg/mL, or at least 180 mg/mL, or at a concentration of 140 mg/mL to 220 mg/mL.

3. (canceled)

4. The formulation of claim 1, wherein the formulation is an aqueous formulation.

5. The formulation of claim 1, wherein:
(i) the organic acid buffer is a histidine buffer;
(ii) the non-ionic surfactant is selected from the group consisting of polysorbate 80, polysorbate and poloxamer 188;
(iii) the non-ionic surfactant is polysorbate 80; and/or
(iv) the at least one amino acid stabiliser includes proline and/or arginine.

6. The formulation of claim 1, wherein:
(i) the organic acid buffer is present in the formulation at a concentration of 10 to 30 mM; and/or
(ii) the non-ionic surfactant is present in the formulation at a concentration of 0.02% (w/v) to 0.05 (w/v).

7. (canceled)

8. (canceled)

9. (canceled)

10. (canceled)

11. The formulation of claim 1, wherein:
(i) the at least one amino acid stabiliser includes proline, and wherein proline is present in the formulation at a concentration of 50 mM to 150 mM; and/or
(ii) the at least one amino acid stabiliser includes arginine, and wherein arginine is present in the formulation at a concentration of 50 mM to 150 mM.

12. (canceled)

13. The formulation of claim 1, wherein the formulation comprises a histidine buffer, proline and polysorbate 80.

14. The formulation of claim 13, wherein the formulation further comprises arginine.

15. (canceled)

16. The formulation of claim 1, wherein the formulation has:
(i) a pH of 5.3 to 5.7 and comprises 10 mM to 30 mM histidine buffer, 0.02% to 0.04% (w/v) polysorbate 80, 50 mM to 150 mM proline and 50 mM to 150 mM arginine;
(ii) a pH of 5.3 to 5.7 and comprises 15 mM to 25 mM histidine buffer, 0.02% to 0.04% (w/v) polysorbate 80, 90 mM to 110 mM proline and 90 mM to 110 mM arginine; and/or
(iii) a pH of 5.4-5.6 and comprises 20 mM histidine buffer, 0.03% (w/v) polysorbate 80, 100 mM proline and 100 mM arginine.

17. (canceled)

18. (canceled)

19. The formulation of claim 1, wherein the formulation has:
(i) a dynamic viscosity of less than 20 mPa*s at 20° C., less than 10 mPa*s at 20° C., or less than 8 mPa*s at 20° C.; and/or
(ii) an osmolality in the range of 250 mOsm/kg to 450 mOsm/kg.

20. (canceled)

21. The formulation of claim 1, wherein one or more or all of the following apply:
a) the formulation comprises no more than 5% high molecular weight species (HMWS), as determined by size exclusion high performance liquid chromatography (SE-HPLC);
b) at least 95% of the protein in the formulation is a monomer, as determined by SE-HPLC; and
c) the formulation comprises no more than 5% low molecular weight species (LMWS), as determined by capillary electrophoresis with sodium dodecylsulfate (CE-SDS) under non-reducing conditions.

22. The formulation of claim 21, wherein the amount of HMWS, monomer or LMWS is determined after storage for a period of at least 1 month, or at least 2 months, or at least 3 months, or at least 6 months, or at least 9 months, or at least 12 months at a temperature in the range of 2° C. to 35° C.

23. The formulation of claim 1, wherein the formulation has a volume in the range of 0.5 mL to 5 mL.

24. The formulation of claim 1, wherein the protein:
(i) inhibits VEGF-B signaling; and/or
(ii) comprises an antigen binding domain of an antibody; and/or
(iii) is selected from the group consisting of:
a. a single chain Fv fragment (scFv);
b. a dimeric scFv (di-scFv);
c. a diabody;
d. a triabody
e. a tetrabody;
f. a Fab;
g. a F(ab′)2;
h. a Fv;
i. one of (i) to (viii) is linked to a constant region of an antibody, Fc or a heavy chain constant domain (C_{H})2 and/or C_{H}3; and
j. an antibody.

25. (canceled)

26. (canceled)

27. (canceled)

28. The formulation of claim 1, wherein the protein:
(i) competitively inhibits the binding of antibody 2H10 to VEGF-B, wherein the antibody 2H10 comprises a heavy chain variable region (V_{H}) comprising a sequence set forth in SEQ ID NO: 3 and a light chain variable region (V_{L}) comprising a sequence set forth in SEQ ID NO: 4; and/or
(ii) comprises a humanized variable region of antibody 2H10; and/or
(iii) comprises a variable region comprising a V_{H} comprising three complementarity determining regions (CDRs) of a V_{H} comprising an amino acid sequence set forth in SEQ ID NO: 3 and a V_{L} comprising three CDRs of a V_{L} comprising an amino acid sequence set forth in SEQ ID NO: 4; and/or
(iv) comprises a V_{H} comprising:
(a) a CDR1 comprising a sequence set forth in amino acids 25-34 of SEQ ID NO: 3;
(b) a CDR2 comprising a sequence set forth in amino acids 49-65 of SEQ ID NO: 3; and
(c) a CDR3 comprising a sequence set forth in amino acids 98-108 of SEQ ID NO: 3; and
a V_{L} comprising:
(a) a CDR1 comprising a sequence set forth in amino acids 23-33 of SEQ ID NO: 4;
(b) a CDR2 comprising a sequence set forth in amino acids 49-55 of SEQ ID NO: 4; and
(c) a CDR3 comprising a sequence set forth in amino acids 88-96 of SEQ ID NO: 4; and/or
(v) is an antibody comprising a V_{H} comprising a sequence set forth in SEQ ID NO: 5 and a V_{L} comprising a sequence set forth in SEQ ID NO: 6.

29. (canceled)

30. (canceled)

31. (canceled)

32. The formulation of claim 1, wherein the protein comprises an IgG₄ constant region and/or a stabilized IgG₄ constant region.

33. (canceled)

34. A pharmaceutical formulation comprising a protein comprising an antigen binding domain that binds to or specifically binds to VEGF-B, a histidine buffer, polysorbate 80, proline and arginine, wherein the formulation has a pH of 5.0 to 6.0, and wherein the protein comprises a V_{H} comprising an amino acid sequence set forth in SEQ ID NO: 5 and a V_{L} comprising an amino acid sequence set forth in SEQ ID NO: 6.

35. A method of treating or preventing or delaying progression of a disease or condition in a subject, the method comprising administering the formulation of claim 1 to the subject, wherein the disease or condition is selected from the group consisting of nephropathy, a wound, a non-alcoholic fatty liver disease (NAFLD) or complication thereof, a stroke, a wasting disorder, obesity, insulin resistance, diabetes, a cardiovascular disorder, a metabolic syndrome, and combinations thereof.

36. (canceled)

37. (canceled)

38. (canceled)

39. A prefilled syringe comprising the pharmaceutical formulation of claim 1.

40. An autoinjector device comprising the pharmaceutical formulation of claim 1.
